(19) United States
(12) Patent Application Publication
(10) Pub. No.: US 2016/0074399 A1
(43) Pub. Date: Mar. 17, 2016

(54) Salts of an Epidermal Growth Factor Receptor Kinase Inhibitor

(71) Applicant: Clovis Oncology, Inc., Boulder, CO (US)

(72) Inventors: Mei LAI, Longmont, CO (US); Andrew Roger ALLEN, San Francisco, CA (US)

(21) Appl. No.: 14/784,503

(22) PCT Filed: May 5, 2014
(86) PCT No.: PCT/US2014/036743

§ 371 Date: Oct. 14, 2015

Related U.S. Application Data
(60) Provisional application No. 61/926,008, filed on Jan. 10, 2014.
(60) Provisional application No. 61/820,096, filed on May 6, 2013.

(51) Int. Cl. A61K 31/506

(52) U.S. Cl. CPC A61K 31/506

(57) ABSTRACT

The present invention provides a salt form and compositions thereof, which are useful as an inhibitor of EGFR kinases and which exhibits desirable characteristics for the same. Examples include hydrobromide and bis-besylate salts of N-(3-(2-(4-(4-acerylpiperazin-1-yl)-2-methoxyphenylamino)-5-(trifluoromethyl)pyrimidin-4-ylamino)phenyl)acrylamide). The salts and their polymorphs are evaluated for their properties such as stability, solubility, and pharmacokinetics.

### RELATED APPLICATIONS

This application claims the benefit of U.S. Provisional Application Ser. No. 61/820,096, filed May 6, 2013 and U.S. Provisional Application Ser. No. 61/926,008, filed Jan. 10, 2014. Each of these applications is incorporated herein by reference in its entirety for all purposes.

### FIELD OF THE INVENTION

The present invention provides salt forms of a compound useful as mutant-selective inhibitors of epidermal growth factor receptor (EGFR) kinase, including polymorphic forms of certain salts. The invention also provides pharmaceutically acceptable compositions comprising salt forms of the present invention and methods of using the compositions in the treatment of various disorders.

### BACKGROUND OF THE INVENTION

Protein tyrosine kinases are a class of enzymes that catalyze the transfer of a phosphate group from ATP or GTP to a tyrosine residue located on a protein substrate. Receptor tyrosine kinases act to transmit signals from the outside of a cell to the inside by activating secondary messaging effectors via a phosphorylation event. A variety of cellular processes are promoted by these signals, including proliferation, carbohydrate utilization, protein synthesis, angiogenesis, cell growth, and cell survival.

There is strong precedent for involvement of the EGFR in human cancer because over 60% of all solid tumors overexpress at least one of these proteins or their ligands. Overexpression of EGFR is commonly found in breast, lung, head and neck, bladder tumors.

Activating mutations in the tyrosine kinase domain of EGFR have been identified in patients with non-small cell lung cancer (Lin, N. U.; Winer, E. P., Breast Cancer Res 6: 204-210, 2004). The reversible inhibitors Tarceva (erlotinib) and Iressa (gefitinib) currently are first-line therapy for non-small cell lung cancer patients with activating mutations. The most common activating mutations are L858R and delE746-A750.

Additionally, in the majority of patients that relapse, acquired drug resistance, such as by mutation of gatekeeper residue T790M, has been detected in at least half of such clinically resistant patients. Moreover, T790M may also be pre-existing; there may be an independent, oncogenic role for the T790M mutation. For example, there are patients with the L858R/T790M mutation who never received gefitinib treatment. In addition, germline EGFR T790M mutations are linked with certain familial lung cancers.

Current drugs in development, including second-generation covalent inhibitors, such as BIBW2992, HKI-272 and PF-0299804, are effective against the T790M resistance mutation but exhibit dose-limiting toxicities due to concurrent inhibition of WT EGFR. Accordingly, there remains a need to find mutant-selective EGFR kinase inhibitors useful as therapeutic agents.

### SUMMARY OF THE INVENTION

It has now been found that the novel benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid salts of the present invention, and compositions thereof, are useful as mutant-selective inhibitors of one or more EGFR kinases and exhibits desirable characteristics for the same. In general, these salts, and pharmaceutically acceptable compositions thereof, are useful for treating or lessening the severity of a variety of diseases or disorders as described in detail herein.

It has now been found that a useful pharmaceutical dosage form comprises Compound 2:

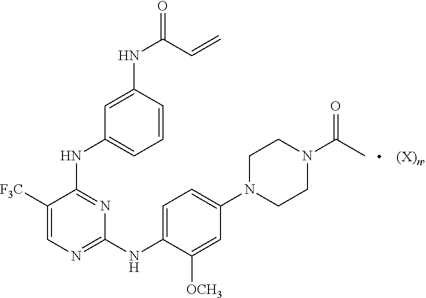

wherein:
n is 1 or 2; and
X is hydrobromic acid, benzenesulfonic acid, camphor sulfonic acid, 1,2-ethane disulfonic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid, wherein the dosage form comprises Compound 2 in an amount of about 50 mg to about 1000 mg.

### BRIEF DESCRIPTION OF THE DRAWINGS

FIG. 1 depicts the x-ray powder diffraction (XRPD) pattern for a bis-besylate salt of Compound 1.

FIG. 2 depicts the thermogravimetric analysis (TGA) pattern for a bis-besylate salt of Compound 1.

FIG. 3 depicts the thermogravimetric analysis (TGA) pattern for a further dried sample of a bis-besylate salt of Compound 1.

FIG. 4 depicts the differential scanning calorimetry (DSC) pattern for a bis-besylate salt of Compound 1.

FIG. 5 depicts the infrared (IR) spectrum of a bis-besylate salt of Compound 1.

FIG. 6 depicts the ¹H-NMR spectrum of a bis-besylate salt of Compound 1.

FIG. 7 depicts the dynamic vapor sorption (DVS) pattern of a bis-besylate salt of Compound 1.

FIG. 8 depicts the results of a hydration study of a bis-besylate salt of Compound 1, as analyzed by the XRPD patterns.

FIG. 9 depicts the results of a disproportionation study of a bis-besylate salt of Compound 1, as analyzed by the XRPD patterns.

FIG. 10 depicts the results of a stability study of a bis-besylate salt of Compound 1, as analyzed by the XRPD patterns.

FIG. 11 depicts the results of a thermodynamic solubility study of a bis-besylate salt of Compound 1, as analyzed by the XRPD patterns.

FIG. 12 depicts the dissolution at pH 4.5 of a compressed disc of a bis-besylate salt of Compound 1.

FIG. 13 depicts the dissolution at pH 3.0 of a compressed disc of a bis-besylate salt of Compound 1.

FIG. 14 depicts the XRPD pattern for a bis-besylate hydrate salt of Compound 1.

FIG. 15 depicts the TGA pattern for a bis-besylate hydrate salt of Compound 1.

FIG. 16 depicts the DSC pattern for a bis-besylate hydrate salt of Compound 1.

FIG. 17 depicts the IR spectrum of a bis-besylate hydrate salt of Compound 1.

FIG. 18 depicts the ¹H-NMR spectrum of a bis-besylate hydrate salt of Compound 1.

FIG. 19 depicts the DVS pattern of a bis-besylate hydrate salt of Compound 1.

FIG. 20 depicts the results of a stability study of a bis-besylate hydrate salt of Compound 1, as analyzed by the XRPD patterns.

FIG. 21 depicts the results of a thermodynamic solubility study of a bis-besylate salt of Compound 1, as analyzed by the XRPD patterns.

FIG. 22 depicts the dissolution at pH 4.5 of a compressed disc of a bis-besylate hydrate salt of Compound 1.

FIG. 23 depicts the dissolution at pH 3.0 of a compressed disc of a bis-besylate hydrate salt of Compound 1.

FIG. 24 depicts the XRPD pattern for a mono-maleate salt of Compound 1.

FIG. 25 depicts the TGA pattern for a mono-maleate salt of Compound 1.

FIG. 26 depicts the DSC pattern for a mono-maleate salt of Compound 1.

FIG. 27 depicts the ¹H-NMR spectrum of a mono-maleate salt of Compound 1.

FIG. 28 depicts the XRPD pattern for a bis-hydrochloride salt of Compound 1.

FIG. 29 depicts the TGA pattern for a bis-hydrochloride salt of Compound 1.

FIG. 30 depicts the DSC pattern for a bis-hydrochloride salt of Compound 1.

FIG. 31 depicts the ¹H-NMR spectrum of a bis-hydrochloride salt of Compound 1.

FIG. 32 depicts the XRPD pattern for a Form I hydrobromide salt of Compound 1.

FIG. 33 depicts the TGA pattern for a Form I hydrobromide salt of Compound 1.

FIG. 34 depicts the DSC pattern for a Form I hydrobromide salt of Compound 1.

FIG. 35 depicts the IR spectrum of a Form I hydrobromide salt of Compound 1.

FIG. 36 depicts the ¹H-NMR spectrum of a Form I hydrobromide salt of Compound 1.

FIG. 37 depicts the DVS pattern of a Form I hydrobromide salt of Compound 1.

FIG. 38 depicts the results of a hydration study of a Form I hydrobromide salt of Compound 1, as analyzed by the XRPD patterns.

FIG. 39 depicts the results of a disproportionation study of a Form I hydrobromide salt of Compound 1, as analyzed by the XRPD patterns.

FIG. 40 depicts the results of a stability study of a Form I hydrobromide salt of Compound 1, as analyzed by the XRPD patterns.

FIG. 41 depicts the results of a thermodynamic solubility study of a Form I hydrobromide salt of Compound 1, as analyzed by the XRPD patterns.

FIG. 42 depicts the dissolution at pH 4.5 of a compressed disc of a Form I hydrobromide salt of Compound 1.

FIG. 43 depicts the dissolution at pH 3.0 of a compressed disc of a Form I hydrobromide salt of Compound 1.

FIG. 44 depicts the XRPD pattern for a Form I hydrobromide salt of Compound 1.

FIG. 45 depicts the TGA pattern for a Form I hydrobromide salt of Compound 1.

FIG. 46 depicts the IR spectrum of a Form I hydrobromide salt of Compound 1.

FIG. 47 depicts the XRPD pattern for a Form I hydrobromide salt of Compound 1.

FIG. 48 depicts the ¹H-NMR spectrum for a Form I hydrobromide salt of Compound 1.

FIG. 49 depicts the DSC pattern of a Form I hydrobromide salt of Compound 1.

FIG. 50 depicts the results of a slurry experiment involving a form of the free base of Compound 1 and a bis-besylate hydrate.

FIG. 51 depicts the results of a slurry experiment involving a Form I hydrobromide salt of Compound 1 at pH 6.2.

FIG. 52 depicts the XRPD pattern for a Form I hydrobromide salt of Compoundl.

FIG. 53 depicts the XRPD pattern for a Form I hydrobromide salt of Compound 1.

FIG. 54 depicts the IR spectrum of a Form I hydrobromide salt of Compound 1.

FIG. 55 depicts the ¹H-NMR spectrum of a Form I hydrobromide salt of Compound 1.

FIG. 56 depicts the TGA pattern for a Form I hydrobromide salt of Compound 1.

FIG. 57 depicts the DSC pattern for a Form I hydrobromide salt of Compound 1.

FIG. 58 depicts the XRPD pattern for a Form I hydrobromide salt of Compound 1 after heating.

FIG. 59 depicts the XRPD pattern for a Form I hydrobromide salt of Compound 1.

FIG. 60 depicts the XRPD pattern for a Form I hydrobromide salt of Compound 1.

FIG. 61 depicts the IR spectrum of a Form I hydrobromide salt of Compound 1.

FIG. 62 depicts the ¹H-NMR spectrum of a Form I hydrobromide salt of Compound 1.

FIG. 63 depicts the TGA pattern for a Form I hydrobromide salt of Compound 1.

FIG. 64 depicts the DSC pattern for a Form I hydrobromide salt of Compound 1.

FIG. 65 depicts the results of a thermodynamic solubility study of a Form I hydrobromide salt of Compound 1, as analyzed by the XRPD patterns.

FIG. 66 depicts the XRPD pattern for a Form I hydrobromide salt of Compound 1, after storage for 1.5 months.

FIG. 67 depicts the XRPD pattern for a Form III hydrobromide salt of Compound 1.

FIG. 68 depicts the TGA pattern for a Form III hydrobromide salt of Compound 1.

FIG. 69 depicts the DSC pattern for a Form III hydrobromide salt of Compound 1.

FIG. 70 depicts the IR spectrum for a Form III hydrobromide salt of Compound 1.

FIG. 71 depicts the ¹H-NMR spectrum of a Form III hydrobromide salt of Compound 1.

FIG. 72 depicts the XRPD pattern for a Form IV hydrobromide salt of Compound 1.

FIG. 73 depicts the XRPD pattern for a Form V hydrobromide salt of Compound 1.

FIG. 74 depicts the XRPD pattern for a Form VI hydrobromide salt of Compound 1.

FIG. 75 depicts the XRPD pattern for a Form VII hydrobromide salt of Compound 1.

FIG. 76 depicts the XRPD pattern for a Form VIII hydrobromide salt of Compound 1.

FIG. 77 depicts the TGA pattern for a Form VIII hydrobromide salt of Compound 1.

FIG. 78 depicts the DSC pattern for a Form VIII hydrobromide salt of Compound 1.

FIG. 79 depicts the XRPD of an amorphous hydrobromide salt of Compound 1.

FIG. 80 depicts the XRPD of Form V hydrobromide salt of Compound 1 following desolvation conditions.

FIG. 81 depicts input material Form I hydrobromide salt of Compound 1 compared with a wet sample, and after stages of drying.

FIG. 82 depicts the XRPD analysis of hydrobromide salt Forms I and III resulting from competitive slurry experiments at ambient temperature (22° C.).

FIG. 83 depicts the XRPD analysis of hydrobromide salt Forms I and III resulting from competitive slurry experiments at 60° C.

FIG. 84 depicts the XRPD analysis of Form I hydrobromide salt of Compound 1 slurried in EtOH:water mixtures.

FIG. 85 depicts the XRPD analysis of material slurried in IPA/acetone (9:1): water mixtures.

FIG. 86 depicts the XRPD analysis following hydration studies at 15° C. and 35° C.

FIG. 87 depicts the form diagram for the hydrobromide salt, including 7 different forms and the relationship between such forms.

FIG. 88 depicts the pharmacokinetic properties of individual and mean CO-1686 Cmax following a single dose of 900 mg CO-1686 free base (FB) (Compound 1) vs. 500 mg CO-1686 HBr (HBr)(Compound 2).

### DETAILED DESCRIPTION OF THE INVENTION

### General Description of Certain Aspects of the Invention

U.S. application Ser. No. 13/286,061, published as US 2012/0149687 on Jun. 14, 2012 (“the '061 application”), filed Oct. 31, 2011, the entirety of which is hereby incorporated herein by reference, describes certain 2,4-disubstituted pyrimidine compounds which covalently and irreversibly inhibit activity of EGFR kinase. Such compounds include compound 1:

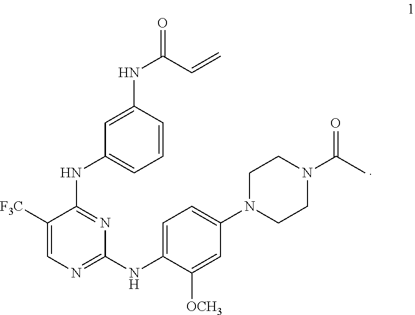 [SMILES: C=CC(=O)Nc1cccc(Nc2nc(Nc3ccc(N4CCN(C(C)=O)CC4)cc3OC)ncc2C(F)(F)F)c1]

Compound 1 (N-(3-(2-(4-(4-acetylpiperazin-1-yl)-2-methoxyphenylamino)-5-(trifluoromethyl)pyrimidin-4-ylamino)phenyl)acrylamide)) is designated as compound number 1-4 and the synthesis of compound 1 is described in detail at Example 3 of the '061 application.

Compound 1 is active in a variety of assays and therapeutic models demonstrating selective covalent, irreversible inhibition of mutant EGFR kinase (in enzymatic and cellular assays). Notably, compound 1 was found to inhibit human non-small cell lung cancer cell proliferation both in vitro and in vivo. Accordingly, compound 1 and its salts are useful for treating one or more disorders associated with activity of mutant EGFR kinase.

It would be desirable to provide a form of compound 1 that, as compared to compound 1, imparts characteristics such as improved aqueous solubility, stability and ease of formulation. Accordingly, the present invention provides several salts of compound 1.

According to one embodiment, the present invention provides a salt of compound 1, represented by compound 2:

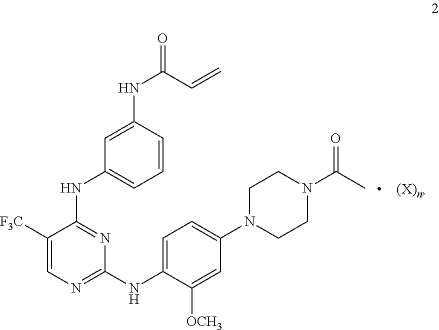

where:

n is 1 or 2; and

X is benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid.

It will be appreciated by one of ordinary skill in the art that the acid moiety indicated as “X” and compound 1 are ionically bonded to form compound 2. It is contemplated that compound 2 can exist in a variety of physical forms. For example, compound 2 can be in solution, suspension, or in solid form. In certain embodiments, compound 2 is in solid form. When compound 2 is in solid form, said compound may be amorphous, crystalline, or a mixture thereof. Exemplary solid forms are described in more detail below.

In other embodiments, the present invention provides compound 2 substantially free of impurities. As used herein, the term “substantially free of impurities” means that the compound contains no significant amount of extraneous matter. Such extraneous matter may include excess acid “X”, excess compound 1, residual solvents, or any other impurities that may result from the preparation of, and/or isolation of, compound 2. In certain embodiments, at least about 90% by weight of compound 2 is present. In certain embodiments, at least about 95% by weight of compound 2 is present. In still other embodiments of the invention, at least about 99% by weight of compound 2 is present.

According to one embodiment, compound 2 is present in an amount of at least about 95, 97, 97.5, 98.0, 98.5, 99, 99.5, 99.8 weight percent where the percentages are based on the total weight of the composition. According to another embodiment, compound 2 contains no more than about 5.0 area percent HPLC of total organic impurities and, in certain embodiments, no more than about 3.0 area percent HPLC of total organic impurities and, in certain embodiments, no more than about 1.5 area percent HPLC total organic impurities relative to the total area of the HPLC chromatogram. In other embodiments, compound 2 contains no more than about 1.0 area percent HPLC of any single impurity; no more than about 0.6 area percent HPLC of any single impurity, and, in certain embodiments, no more than about 0.5 area percent HPLC of any single impurity, relative to the total area of the HPLC chromatogram.

The structure depicted for compound 2 is also meant to include all tautomeric forms of compound 2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this invention.

According to one embodiment, the present invention provides a pharmaceutical dosage form comprising Compound 2:

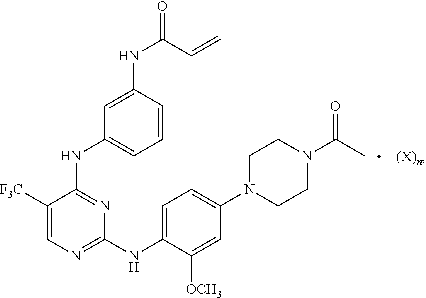

wherein:
n is 1 or 2; and
X is hydrobromic acid, benzenesulfonic acid, camphor sulfonic acid, 1,2-ethane disulfonic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid,
wherein the dosage form comprises Compound 2 in an amount of about 50 mg to about 1000 mg.

According to one embodiment of the invention, X is hydrobromic acid.

According to one embodiment of the invention, Compound 2 is a Form I hydrobromic acid salt characterized by one or more peaks in a powder X-ray diffraction pattern selected from those at about 17.39, about 19.45, about 21.41, about 23.56 and about 27.45 degrees 2-theta.

According to one embodiment of the invention, the total daily dose of Compound 2 is about 500 mg to about 2000 mg.

According to one embodiment of the invention, the dose of Compound 2 is 250 mg BID to 1000 mg BID.

According to one embodiment of the invention, the dose of Compound 2 is 500 mg BID to 750 mg BID.

According to one embodiment of the invention, the dose of Compound 2 is 500 mg BID.

According to one embodiment of the invention, the dose of Compound 2 is 625 mg BID.

According to one embodiment of the invention, the dose of Compound 2 is 750 mg BID.

According to one embodiment of the invention, the dosage form comprises Compound 2 in an amount of about 50 mg to about 500 mg.

According to one embodiment of the invention, the dosage form comprises Compound 2 in an amount of about 125 mg to about 250 mg.

### Solid Forms of Compound 2:

It has been found that compound 2 can exist in a variety of solid forms. Such forms include polymorphs and amorphous forms. The solid forms can be solvates, hydrates and unsolvated forms of compound 2. All such forms are contemplated by the present invention. In certain embodiments, the present invention provides compound 2 as a mixture of one or more solid forms of compound 2.

As used herein, the term “polymorph” refers to the different crystal structures (of solvated or unsolvated forms) in which a compound can crystallize.

As used herein, the term “solvate” refers to a crystal form with either a stoichiometric or non-stoichiometric amount of solvent. For polymorphs, the solvent is incorporated into the crystal structure. Similarly, the term “hydrate” refers to a solid form with either a stoichiometric or non-stoichiometric amount of water. For polymorphs, the water is incorporated into the crystal structure.

As used herein, the term “about”, when used in reference to a degree 2-theta value refers to the stated value±0.3 degree 2-theta (° 20). In certain embodiments, “about” refers to ±0.2 degree 2-theta or ±0.1 degree 2-theta.

In certain embodiments, compound 2 is a crystalline solid. In other embodiments, compound 2 is a crystalline solid substantially free of amorphous compound 2. As used herein, the term “substantially free of amorphous compound 2” means that the compound contains no significant amount of amorphous compound 2. In certain embodiments, at least about 90% by weight of crystalline compound 2 is present, or at least about 95% by weight of crystalline compound 2 is present. In still other embodiments of the invention, at least about 99% by weight of crystalline compound 2 is present.

In certain embodiments, compound 2 is a benzenesulfonic acid (besylate) salt. The salt can be a mono-besylate or a bis-besylate. A besylate salt is optionally solvated or hydrated, such as a monohydrate.

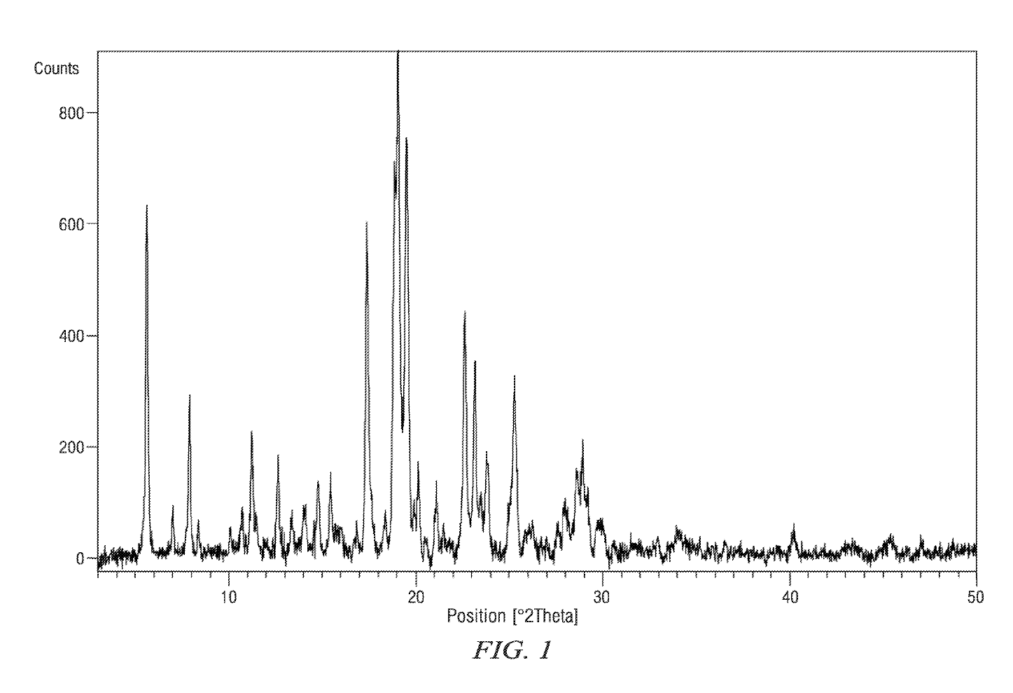

According to one aspect, an unsolvated bis-besylate salt has a powder X-ray diffraction pattern substantially similar to that depicted in FIG. 1. According to one embodiment, an unsolvated bis-besylate salt is characterized by one or more peaks in its powder X-ray diffraction pattern selected from those at about 5.62, about 17.41, about 18.90, about 19.07 and about 19.52 degrees 2-theta. In some embodiments, an unsolvated bis-besylate salt is characterized by two or more peaks in its powder X-ray diffraction pattern selected from those at about 5.62, about 17.41, about 18.90, about 19.07 and about 19.52 degrees 2-theta. In certain embodiments, an unsolvated bis-besylate salt is characterized by three or more peaks in its powder X-ray diffraction pattern selected from those at about 5.62, about 17.41, about 18.90, about 19.07 and about 19.52 degrees 2-theta. In particular embodiments, an unsolvated bis-besylate salt is characterized by substantially all of the peaks in its X-ray powder diffraction pattern selected from those at about 5.62, 7.89, 11.23, 12.64, 17.41, 18.90, 19.07, 19.52, 22.63, 23.17, 25.28 and 28.92 degrees 2-theta. In an exemplary embodiment, an unsolvated bis-besylate salt is characterized by substantially all of the peaks in its X-ray powder diffraction pattern selected from those at about

°2-Theta

3.37
3.70
5.62
7.00
7.89
8.38
9.56
10.09
10.72
10.91
11.23
11.97
12.64
12.98
13.37
14.04
14.56
14.79
15.45
16.08
16.47
16.87
17.41
18.40
18.90
19.07
19.52
19.91
20.16
20.50
21.12
21.48
21.95
22.63
23.17
23.46
23.82
24.33
24.48
24.93
25.28
25.85
26.18
26.73
27.00
27.63
27.91
28.03
28.60
28.92
29.15
29.74
29.94
30.60
31.53
32.13
32.40
32.54
32.77
32.96
34.04
35.18
35.61
35.91
36.07
36.54
36.85
37.28
38.25
38.61
39.06
39.34
40.20
41.33
41.80
41.88
42.89
43.40
43.80
44.84
45.41
46.34
46.69
47.05
47.85
48.75
48.96

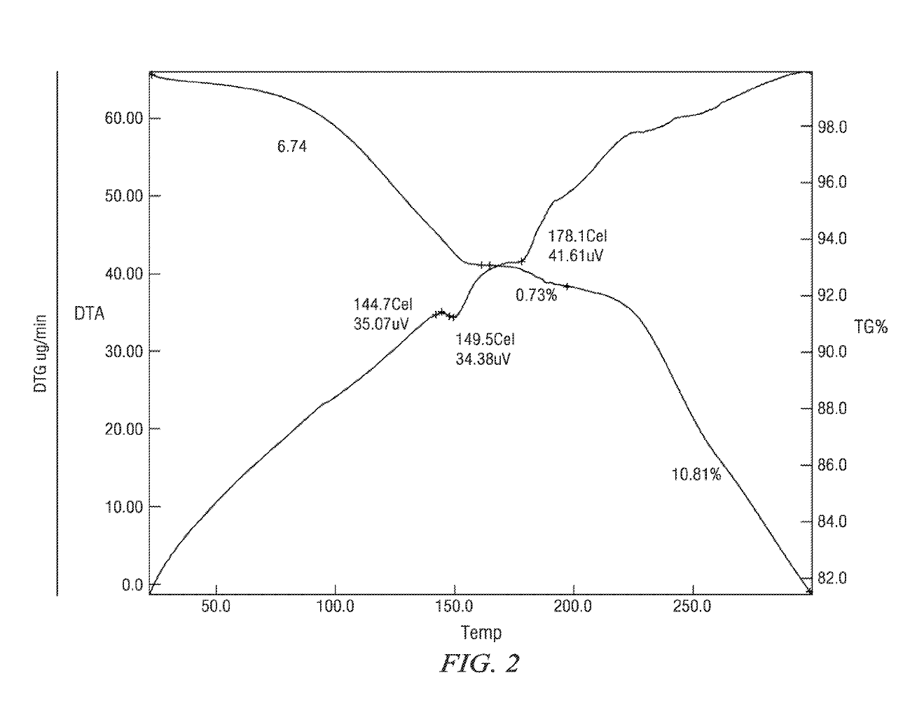

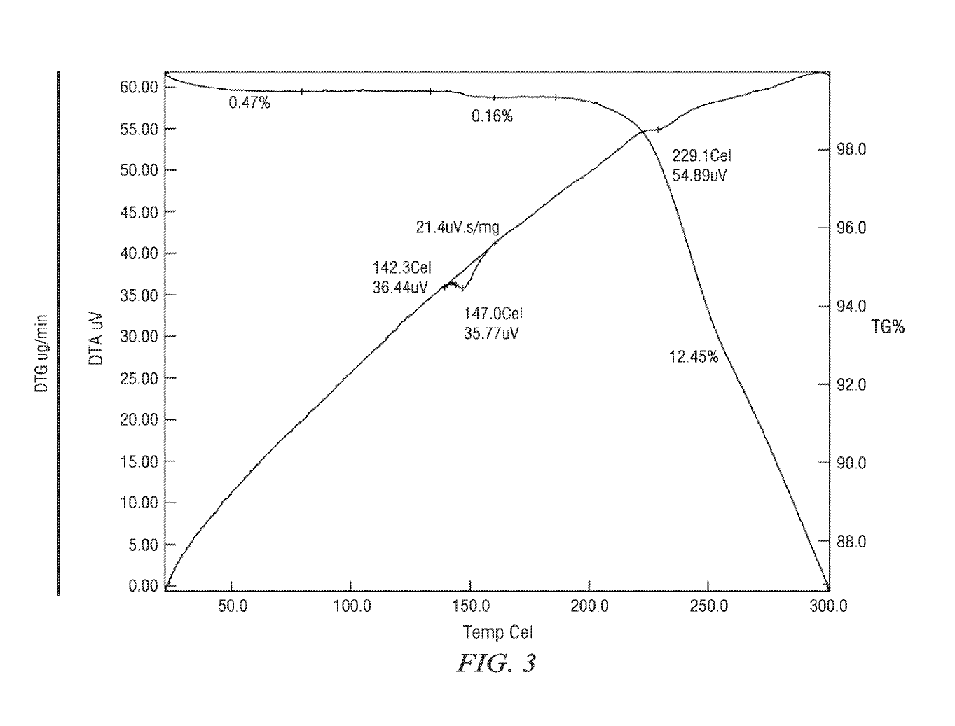

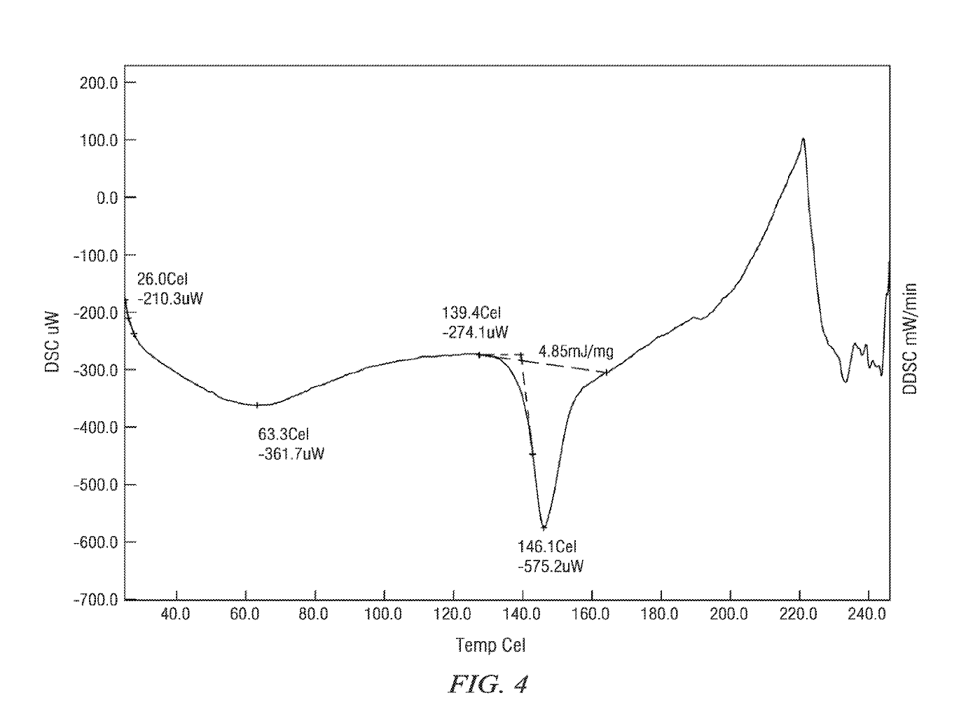

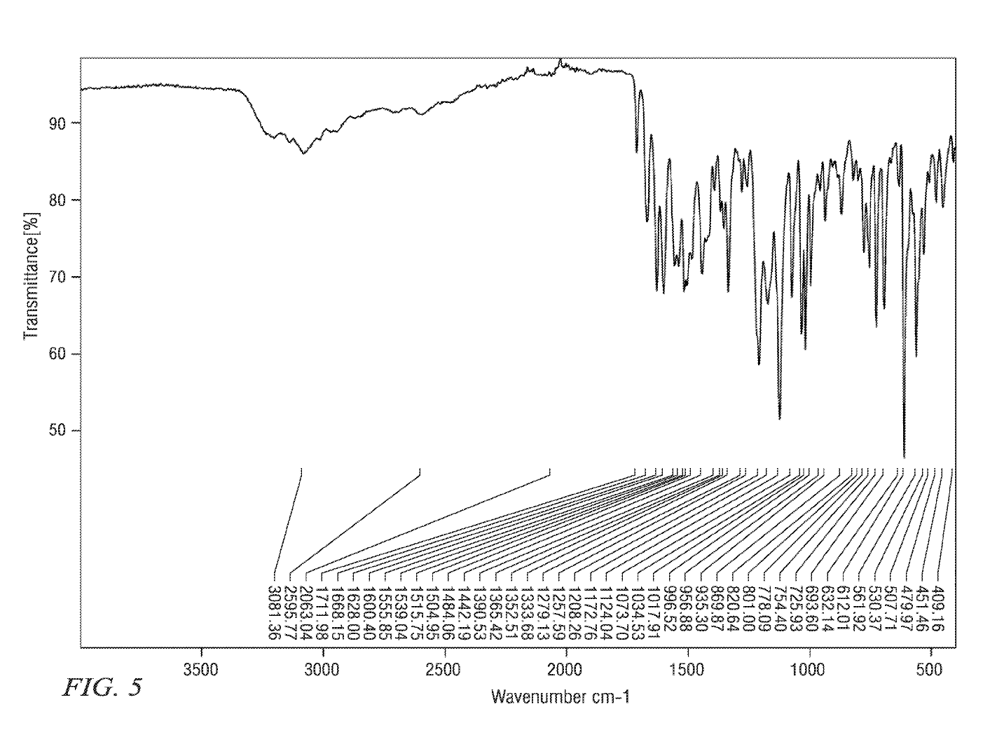

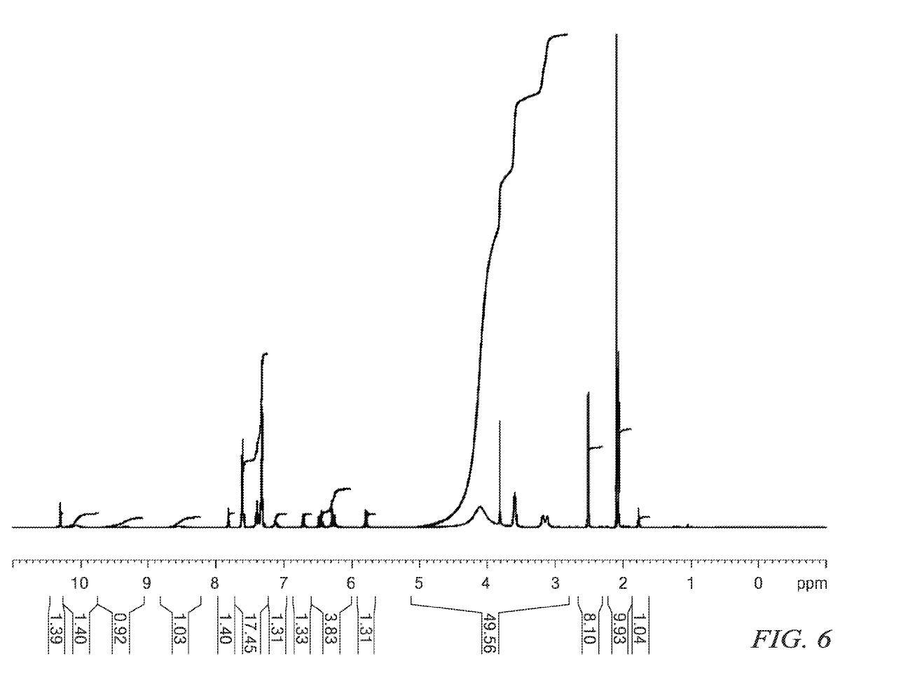

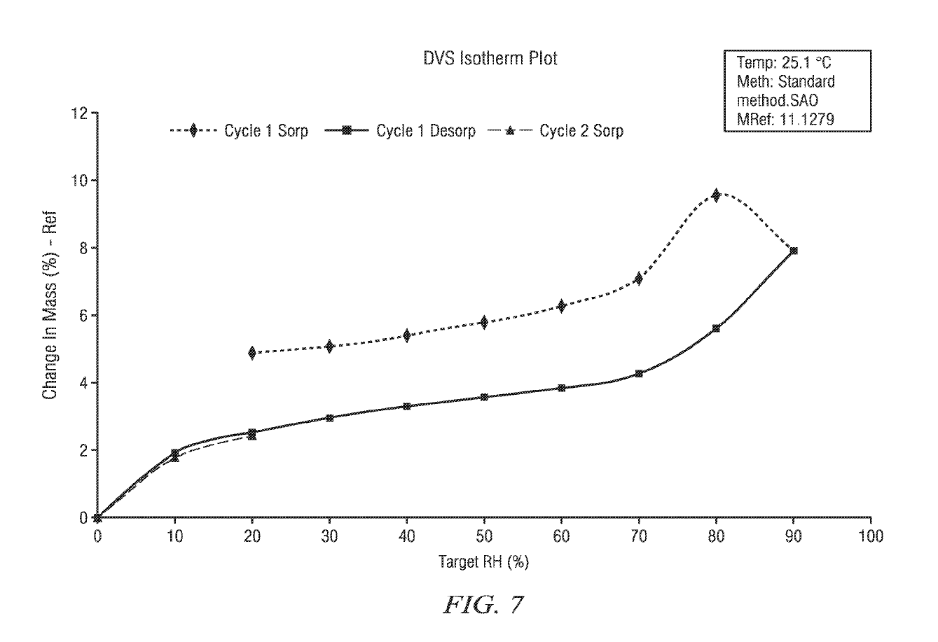

According to another aspect, an unsolvated bis-besylate salt has a thermogravimetric analysis pattern substantially similar to that depicted in FIG. 2 or 3. According to yet another aspect, an unsolvated bis-besylate salt has a differential scanning calorimetry pattern substantially similar to that depicted in FIG. 4. According to a further embodiment, an unsolvated bis-besylate salt has an infrared spectrum substantially similar to that depicted in FIG. 5. According to another embodiment, an unsolvated bis-besylate salt has an ¹H-NMR spectrum substantially similar to that depicted in FIG. 6. According to a further embodiment, an unsolvated bis-besylate salt has a dynamic vapour sorption pattern substantially similar to that depicted in FIG. 7. An unsolvated bis-besylate salt can be characterized by substantial similarity to two or more of these figures simultaneously.

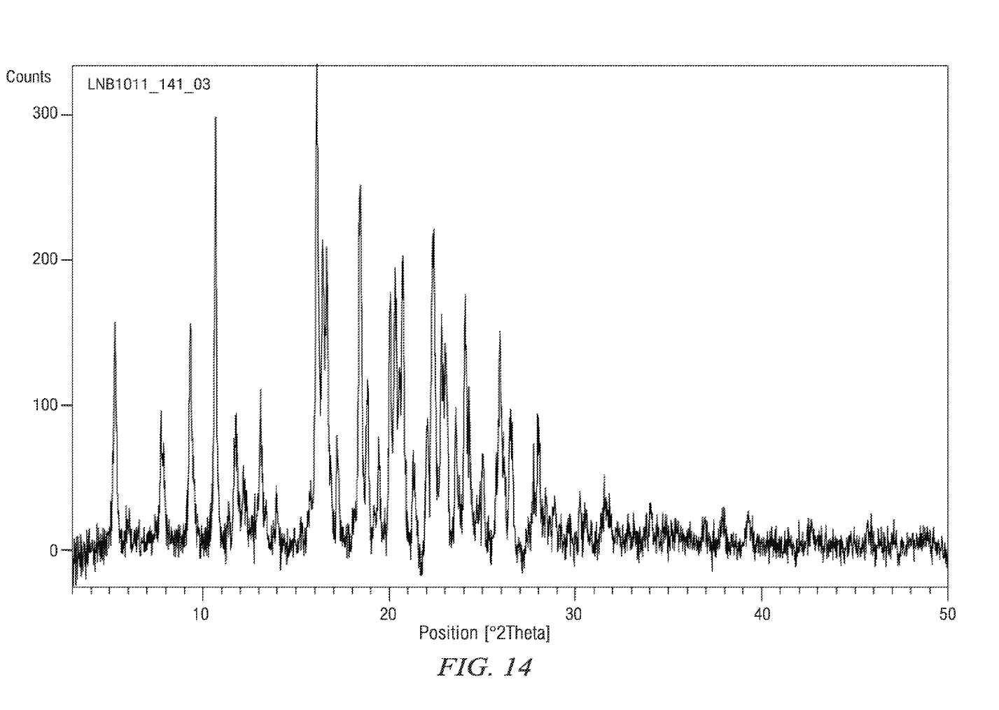

According to one aspect, a bis-besylate hydrate has a powder X-ray diffraction pattern substantially similar to that depicted in FIG. 14. According to one embodiment, a bis-besylate hydrate salt is characterized by one or more peaks in its powder X-ray diffraction pattern selected from those at about 10.68, about 16.10, about 18.44 and about 22.36 degrees 2-theta. In some embodiments, a bis-besylate hydrate salt is characterized by two or more peaks in its powder X-ray diffraction pattern selected from those at about 10.68, about 16.10, about 18.44 and about 22.36 degrees 2-theta. In certain embodiments, a bis-besylate hydrate salt is characterized by three or more peaks in its powder X-ray diffraction pattern selected from those at about 10.68, about 16.10, about 18.44 and about 22.36 degrees 2-theta. In particular embodiments, a bis-besylate hydrate salt is characterized by substantially all of the peaks in its X-ray powder diffraction pattern selected from those at about 9.33, 10.68, 16.10, 16.43, 16.64, 18.44, 20.05, 20.32, 20.74, 22.36 and 22.83 degrees 2-theta. In an exemplary embodiment, a bis-besylate hydrate salt is characterized by substantially all of the peaks in its X-ray powder diffraction pattern selected from those at about

°2-Theta

3.05
3.30
3.50
3.64
5.31
6.02
6.89
7.77
7.90
8.86
9.33
10.68
11.38
11.76
12.19
13.12
13.60
13.95
14.62
16.10
16.43
16.64
17.21
17.67
18.06
18.44
18.82
19.45
20.05
20.32
20.74
21.28
22.05
22.36
22.83
23.04
23.58
24.09
25.01
25.92
26.51
27.41
27.73
28.03
28.43
28.86
29.20
29.63
30.29
30.53
30.92
31.66
32.85
33.22
33.96
34.11
36.94
37.87
39.33
42.63
44.95
45.79
46.36
47.16
47.85
48.86
49.09

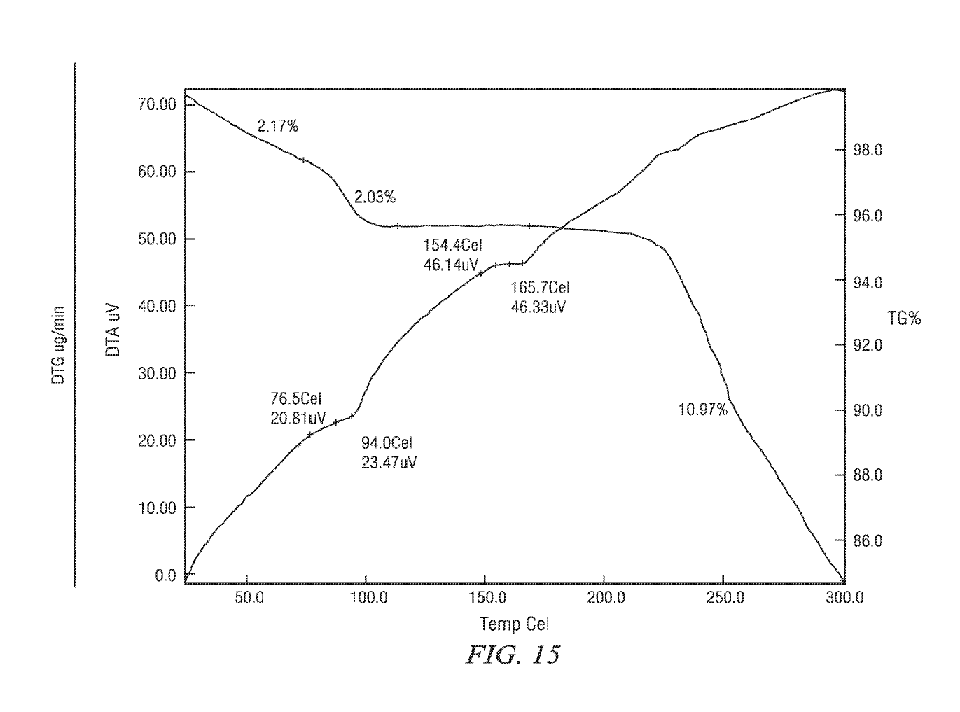

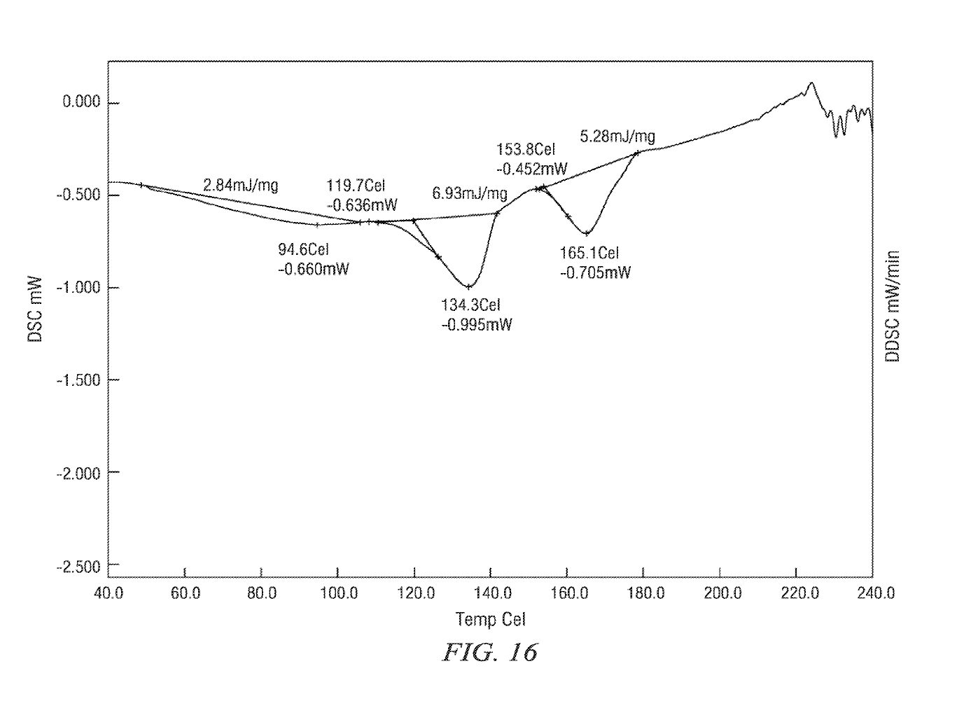

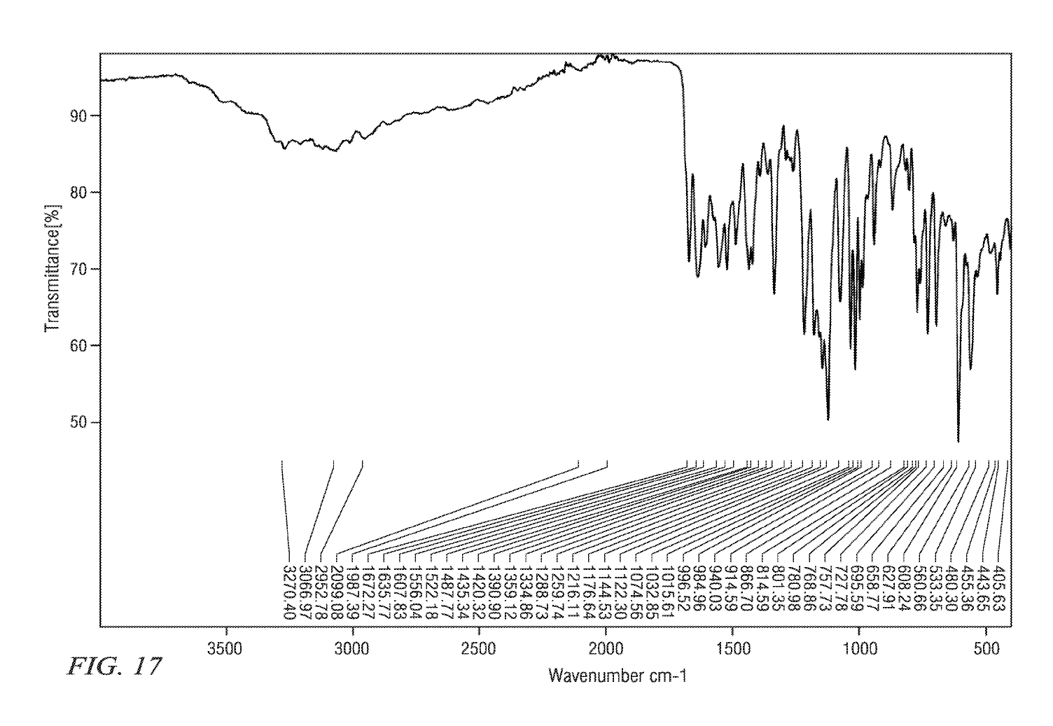

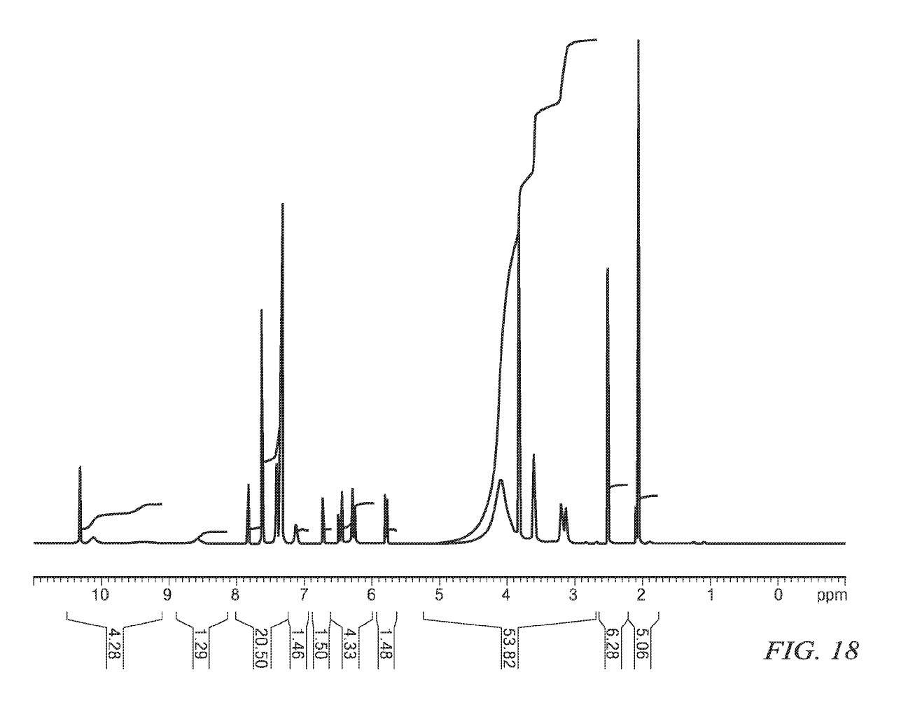

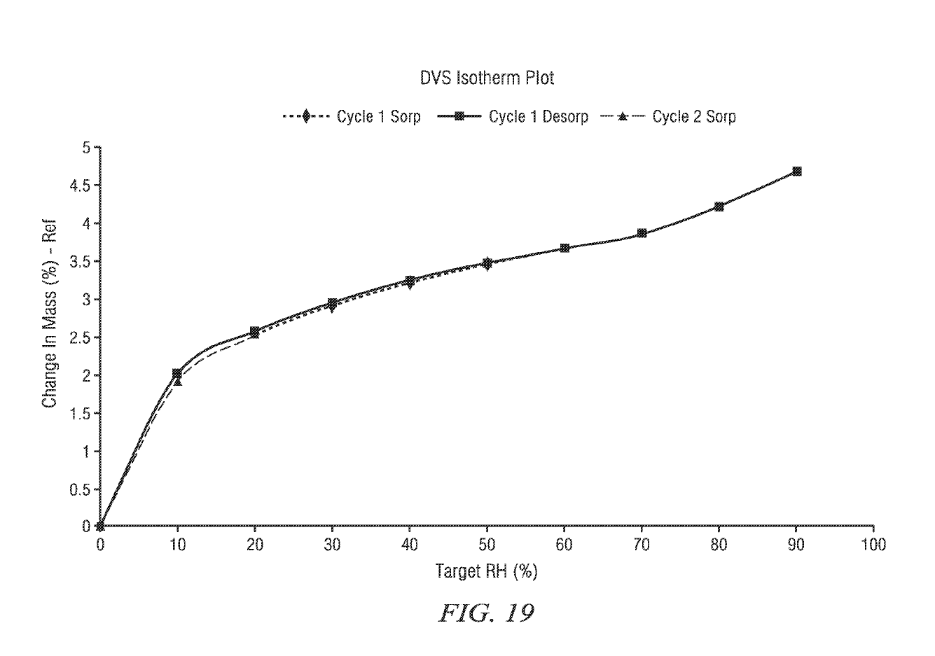

According to another aspect, a bis-besylate hydrate has a thermogravimetric analysis pattern substantially similar to that depicted in FIG. 15. According to yet another aspect, a bis-besylate hydrate has a differential scanning calorimetry pattern substantially similar to that depicted in FIG. 16. According to a further embodiment, a bis-besylate hydrate has a infrared spectrum substantially similar to that depicted in FIG. 17. According to another embodiment, a bis-besylate hydrate has an ¹H-NMR spectrum substantially similar to that depicted in FIG. 18. According to a further embodiment, a bis-besylate hydrate has a dynamic vapour sorption pattern substantially similar to that depicted in FIG. 19. A bis-besylate hydrate can be characterized by substantial similarity to two or more of these figures simultaneously.

In certain embodiments, compound 2 is a camphor sulfonic acid salt (e.g., camphor-10-sulfonic acid). In some embodiments, compound 2 is a mono-camphor sulfonic acid salt. In some embodiments, compound 2 is a bis-camphor sulfonic acid salt.

In certain embodiments, compound 2 is a 1,2-ethane disulfonic acid salt. In some embodiments, compound 2 is a mono-1,2-ethane disulfonic acid salt. In some embodiments, compound 2 is a bis-1,2-ethane disulfonic acid salt.

In certain embodiments, compound 2 is a hydrobromic acid salt. In some embodiments, compound 2 is an anhydrous monohydrobromic acid salt. In some embodiments, compound 2 is an anhydrous bis-hydrobromic acid salt. A hydrobromide salt is optionally solvated or hydrated. In some embodiments, compound 2 is a monohydrate hydrobromic acid salt. In some embodiments, compound 2 is a solvated hydrobromic acid salt. In some such embodiments, the solvate is selected from dimethylsulfoxide (DMSO), dimethylformamide (DMF) and 1,4-dioxane. In some embodiments, compound 2 is a hydrobromide salt selected from Form I, Form III, Form IV, Form V, Form VI, Form VII and Form VIII, each of which is described in further detail, infra.

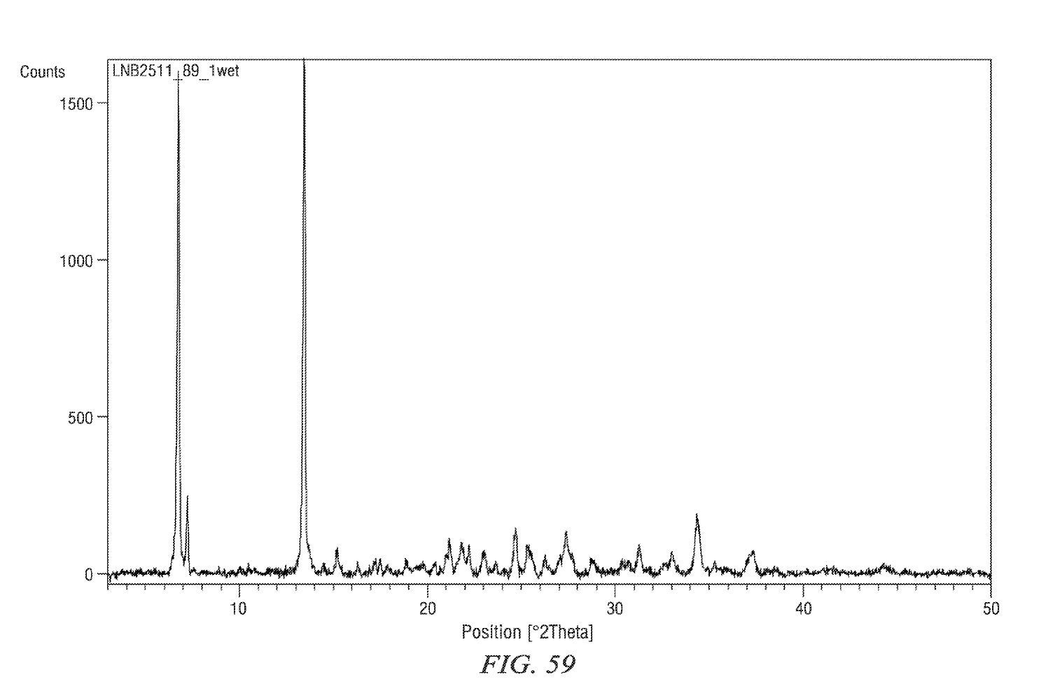

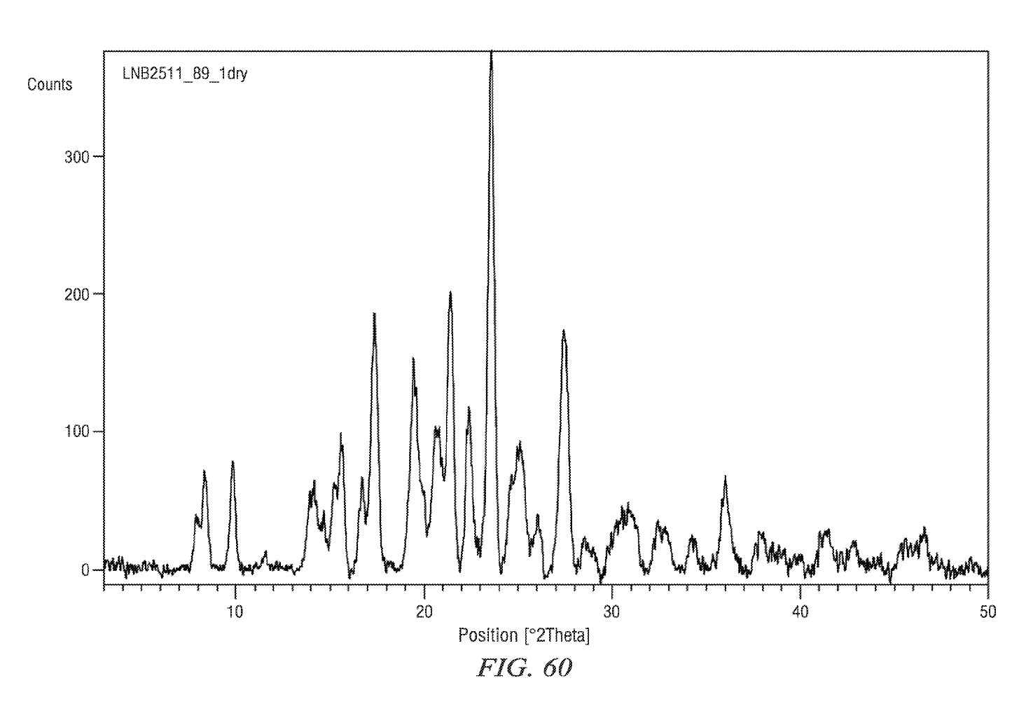

In some embodiments, compound 2 is a Form I hydrobromide salt. In some such embodiments, compound 2 is an anhydrous Form I hydrobromide salt. According to one aspect, a Form I hydrobromide salt is characterized by the powder X-ray diffraction pattern substantially similar to that depicted in FIG. 60. In some embodiments, a Form I hydrobromide salt is characterized by the powder X-ray diffraction pattern substantially similar to that depicted in FIG. 59. According to one embodiment, a Form I mono-hydrobromide salt is characterized by one or more peaks in its powder X-ray diffraction pattern selected from those at about 17.39, about 19.45, about 21.41, about 23.56 and about 27.45 degrees 2-theta. In some embodiments, a Form I mono-hydrobromide salt is characterized by two or more peaks in its powder X-ray diffraction pattern selected from those at about 17.39, about 19.45, about 21.41, about 23.56 and about 27.45 degrees 2-theta. In certain embodiments, a Form I mono-hydrobromide salt is characterized by three or more peaks in its powder X-ray diffraction pattern selected from those at about 17.39, about 19.45, about 21.41, about 23.56 and about 27.45 degrees 2-theta. In some embodiments, a Form I mono-hydrobromide salt is characterized by four or more peaks in its powder X-ray diffraction pattern selected from those at about 17.39, about 19.45, about 21.41, about 23.56 and about 27.45 degrees 2-theta. In particular embodiments, a Form I mono-hydrobromide salt is characterized by an X-ray powder diffraction pattern which includes the peaks at about 9.84, 15.62, 17.39, 19.45, 20.69, 21.41, 22.38, 23.56, 25.08 and 27.45 degrees 2-theta. In an exemplary embodiment, a Form I mono-hydrobromide salt is characterized by substantially all of the peaks in its X-ray powder diffraction pattern selected from those at about

°2-Theta

3.17
3.48
3.79
5.60
7.92
8.35
9.84
11.52
14.10
15.23
15.62
16.73
17.39
18.23
19.45
20.69
21.41
22.38
23.56
24.65
25.08
26.26
27.45
28.50
29.06
29.77
29.94
30.66
31.35
32.45
32.82
34.18
34.80
35.35
36.01
36.82
37.61
37.96
38.55
39.13
40.04
40.64
40.86
41.03
41.39
42.16
42.48
42.78
44.28
45.34
45.59
46.57
47.20
47.51

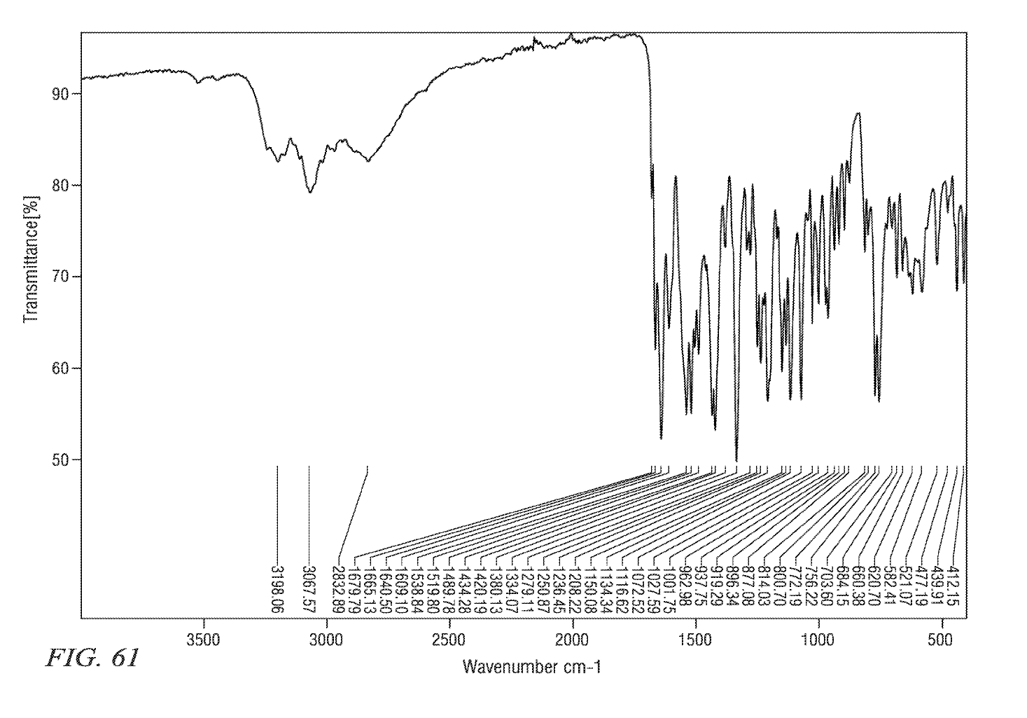

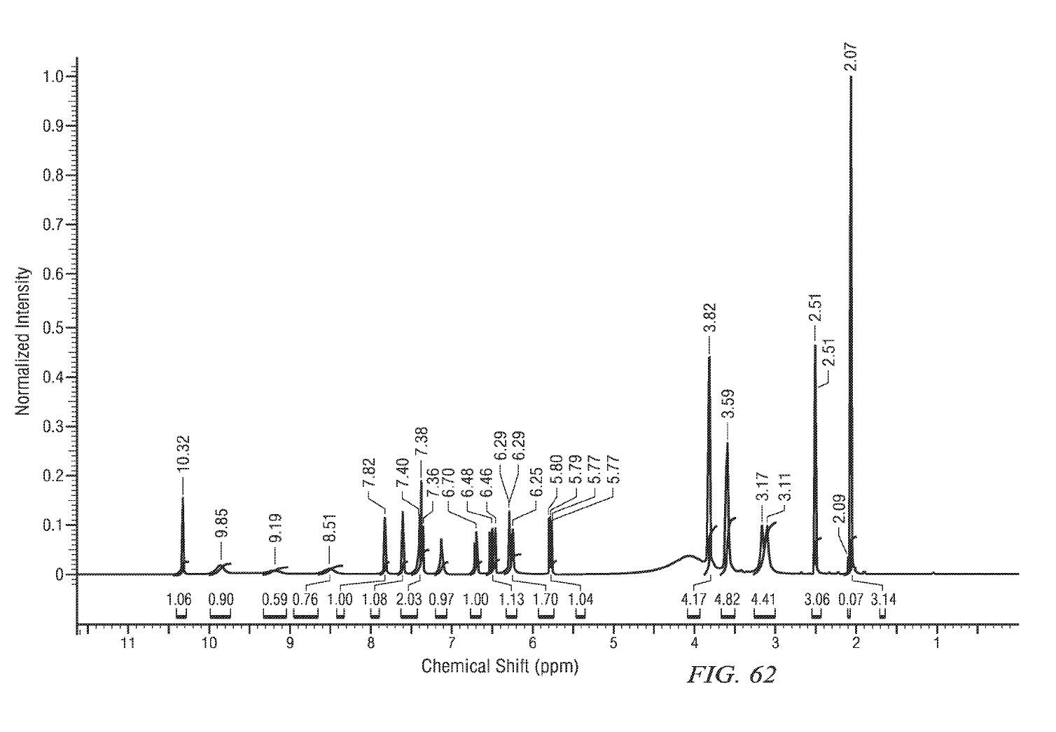

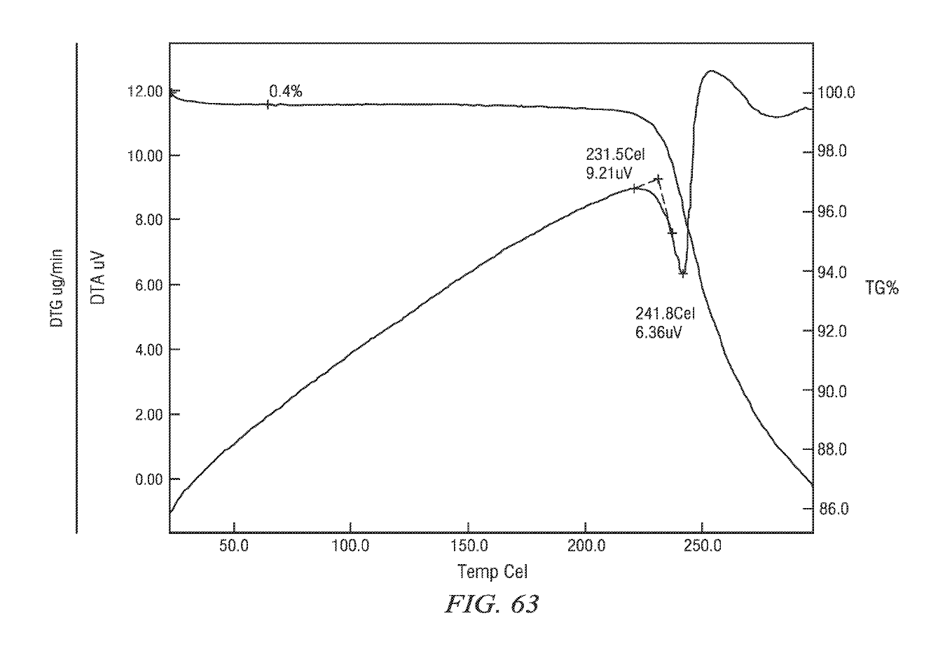

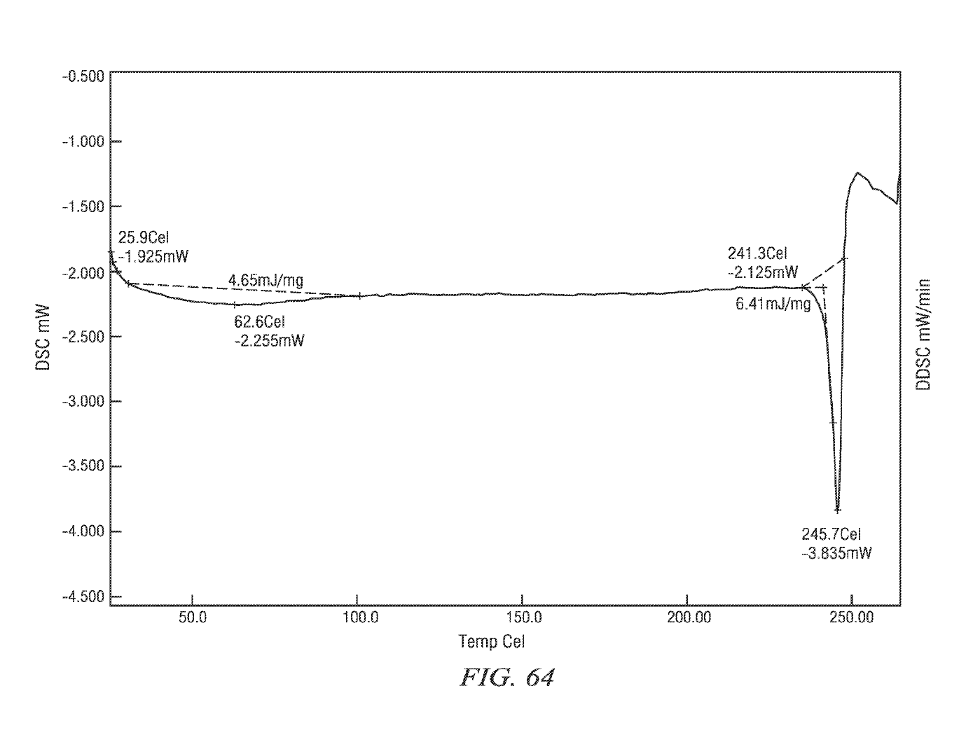

According to another aspect, a Form I mono-hydrobromide salt is characterized by a thermogravimetric analysis pattern substantially similar to that depicted in FIG. 63. According to yet another aspect, a Form I mono-hydrobromide salt is characterized by a differential scanning calorimetry pattern substantially similar to that depicted in FIG. 64. According to a further embodiment, a Form I mono-hydrobromide salt is characterized by an infrared spectrum substantially similar to that depicted in FIG. 61. According to another embodiment, a Form I mono-hydrobromide salt is characterized by a ¹H-NMR spectrum substantially similar to that depicted in FIG. 62. In some embodiments, a Form I mono-hydrobromide salt is characterized by substantial similarity to two or more of these figures simultaneously.

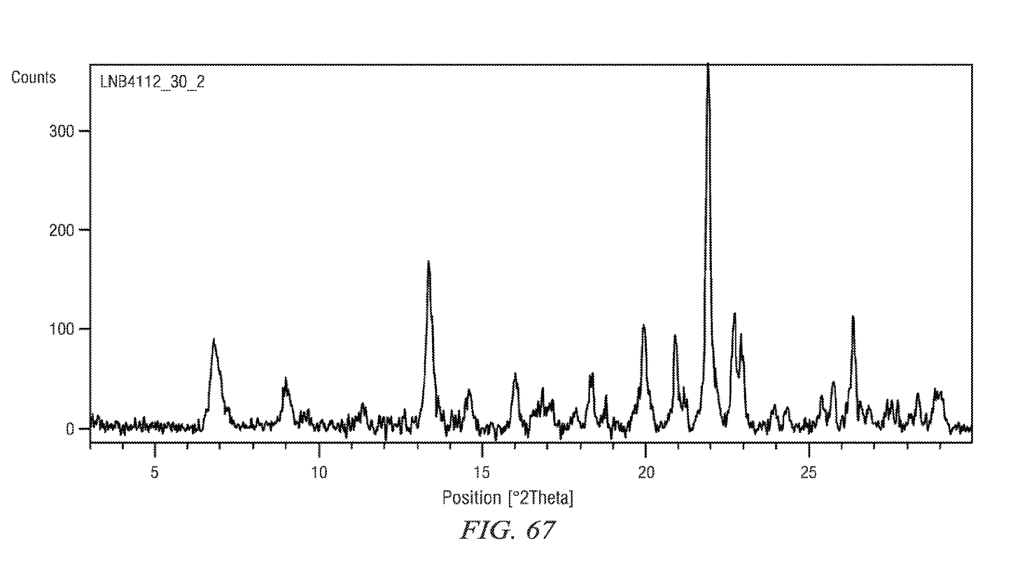

In some embodiments, compound 2 is a Form III hydrobromide salt. In some such embodiments, compound 2 is an anhydrous Form III hydrobromide salt. In some embodiments, a Form III hydrobromide salt is characterized by a powder X-ray diffraction pattern substantially similar to that depicted in FIG. 67. According to one embodiment, a Form III hydrobromide salt is characterized by one or more peaks in its powder X-ray diffraction pattern selected from those at about 6.79, about 13.36, about 19.93, about 20.89, about 21.90, about 22.70, about 22.91 and about 26.34 degrees 2-theta. In some embodiments, a Form III hydrobromide salt is characterized by two or more peaks in its powder X-ray diffraction pattern selected from those at about 6.79, about 13.36, about 19.93, about 20.89, about 21.90, about 22.70, about 22.91 and about 26.34 degrees 2-theta. In certain embodiments, a Form III hydrobromide salt is characterized by three or more peaks in its powder X-ray diffraction pattern selected from those at about 6.79, about 13.36, about 19.93, about 20.89, about 21.90, about 22.70, about 22.91 and about 26.34 degrees 2-theta. In some embodiments, a Form III hydrobromide salt is characterized by four or more peaks in its powder X-ray diffraction pattern selected from those at about 6.79, about 13.36, about 19.93, about 20.89, about 21.90, about 22.70, about 22.91 and about 26.34 degrees 2-theta. In some embodiments, a Form III hydrobromide salt is characterized by five or more peaks in its powder X-ray diffraction pattern selected from those at about 6.79, about 13.36, about 19.93, about 20.89, about 21.90, about 22.70, about 22.91 and about 26.34 degrees 2-theta. In some embodiments, a Form III hydrobromide salt is characterized by six or more peaks in its powder X-ray diffraction pattern selected from those at about 6.79, about 13.36, about 19.93, about 20.89, about 21.90, about 22.70, about 22.91 and about 26.34 degrees 2-theta. In particular embodiments, a Form III hydrobromide salt is characterized by an X-ray powder diffraction pattern which includes the peaks at about 6.79, about 13.36, about 19.93, about 20.89, about 21.90, about 22.70, about 22.91 and about 26.34 degrees 2-theta. In an exemplary embodiment, a Form III hydrobromide salt is characterized by substantially all of the peaks in its X-ray powder diffraction pattern selected from those at about

°2-Theta

6.79
8.15
8.98
9.58
10.36
11.35
13.36
14.06
14.58
15.99
16.67
17.08
17.84
18.33
18.74
19.07
19.93
20.89
21.90
22.70
22.91
23.93
24.32
25.39
25.74
26.34
27.47
28.32
28.89
30.50
30.76
31.39
31.75
32.39
32.68
33.33
33.77
34.48
34.57

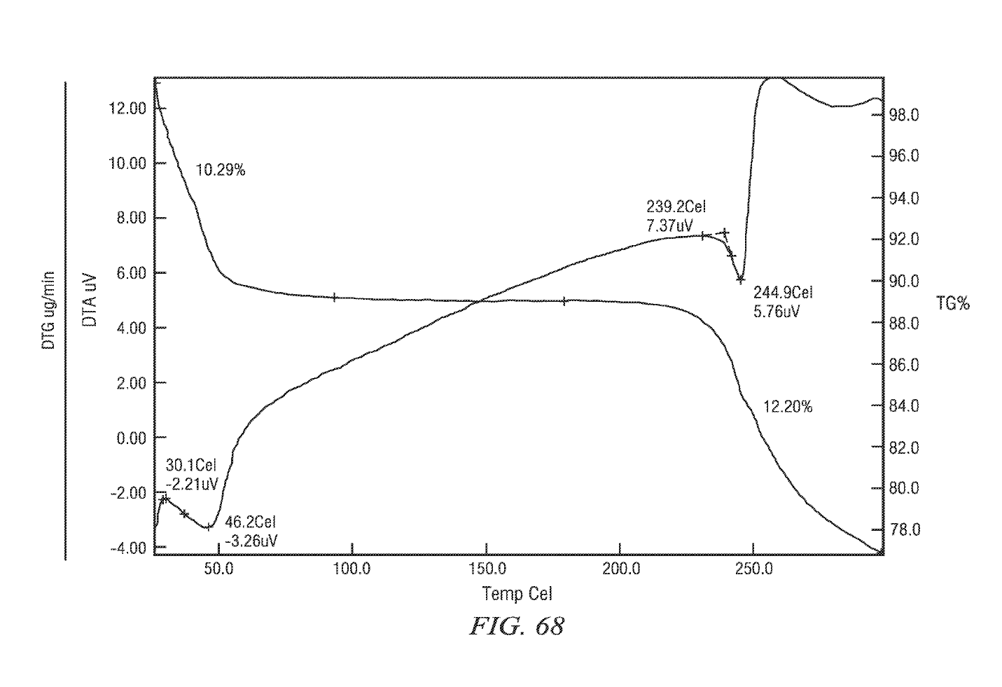

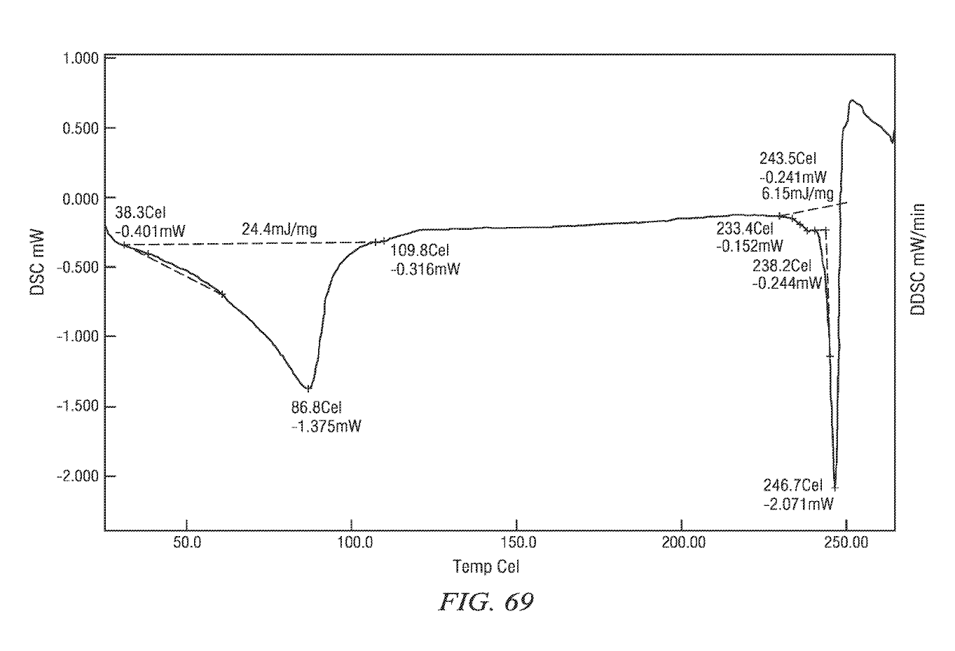

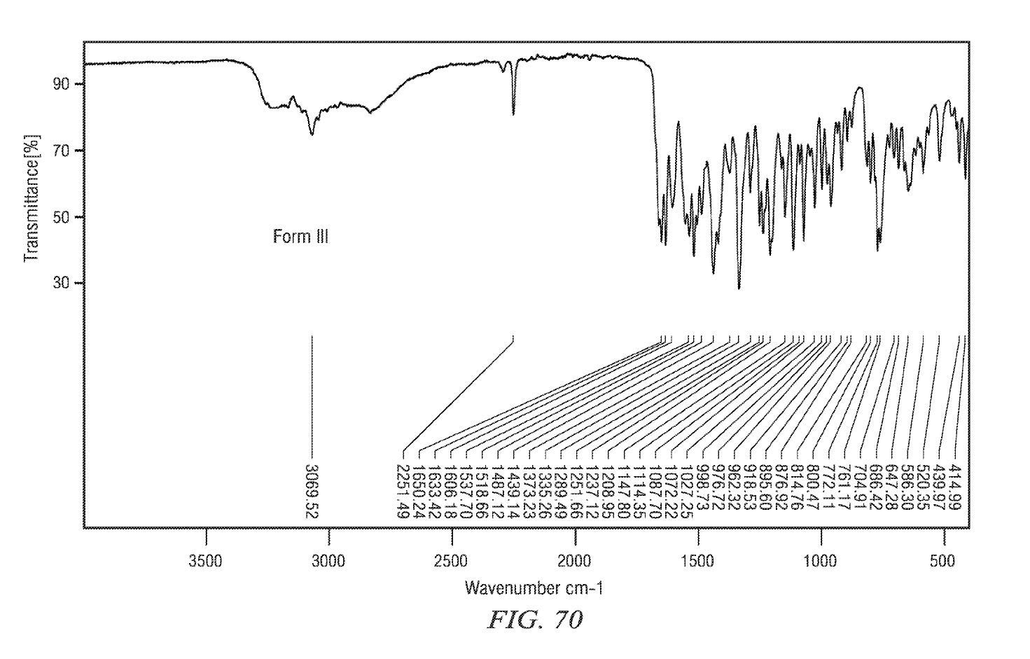

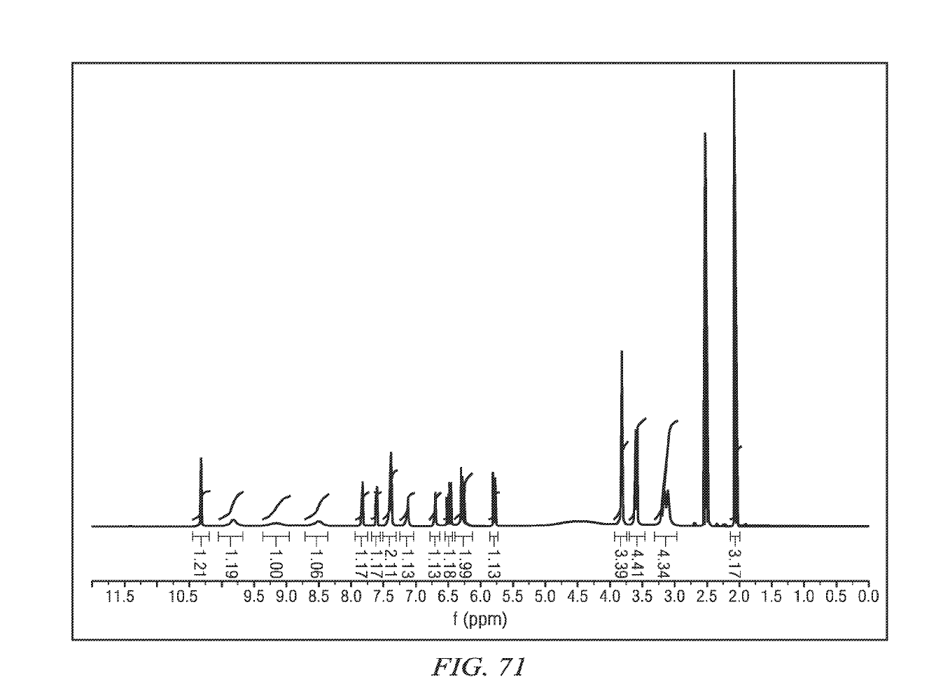

In some embodiments, a Form III hydrobromide salt is characterized by a thermogravimetric analysis pattern substantially similar to that depicted in FIG. 68. In some embodiments, a Form III hydrobromide salt is characterized by a differential scanning calorimetry pattern substantially similar to that depicted in FIG. 69. In some embodiments, a Form III hydrobromide salt is characterized by an infrared spectrum substantially similar to that depicted in FIG. 70. In some embodiments, a Form III hydrobromide salt is characterized by a ¹H-NMR spectrum substantially similar to that depicted in FIG. 71. In some embodiments, a Form III hydrobromide salt is characterized by substantial similarity to two or more of these figures simultaneously.

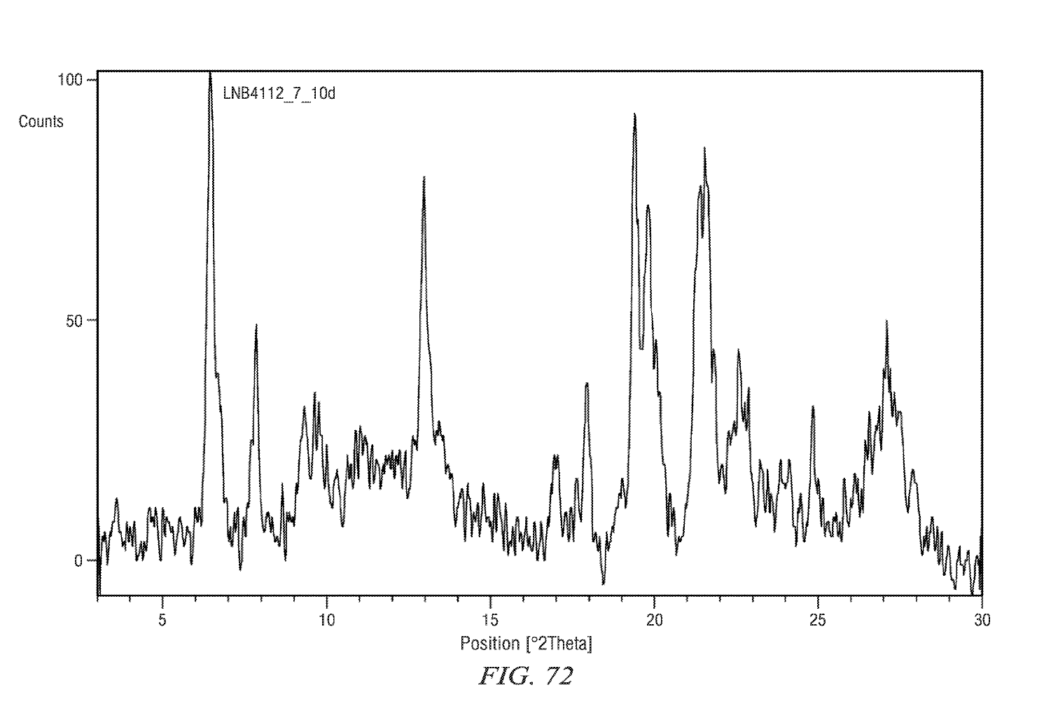

In some embodiments, compound 2 is a Form IV hydrobromide salt. In some such embodiments, a Form IV hydrobromide salt is a 1,4-dioxane solvate. In some embodiments, a Form IV hydrobromide salt is characterized by a powder X-ray diffraction pattern substantially similar to that depicted in FIG. 72. According to one embodiment, a Form IV hydrobromide salt is characterized by one or more peaks in its powder X-ray diffraction pattern selected from those at about 6.45, about 12.96, about 19.38, about 19.79, about 21.37 and about 21.58 degrees 2-theta. In some embodiments, a Form IV hydrobromide salt is characterized by two or more peaks in its powder X-ray diffraction pattern selected from those at about 6.45, about 12.96, about 19.38, about 19.79, about 21.37 and about 21.58 degrees 2-theta. In certain embodiments, a Form IV hydrobromide salt is characterized by three or more peaks in its powder X-ray diffraction pattern selected from those at about 6.45, about 12.96, about 19.38, about 19.79, about 21.37 and about 21.58 degrees 2-theta. In some embodiments, a Form IV hydrobromide salt is characterized by four or more peaks in its powder X-ray diffraction pattern selected from those at about 6.45, about 12.96, about 19.38, about 19.79, about 21.37 and about 21.58 degrees 2-theta. In some embodiments, a Form IV hydrobromide salt is characterized by five or more peaks in its powder X-ray diffraction pattern selected from those at about 6.45, about 12.96, about 19.38, about 19.79, about 21.37 and about 21.58 degrees 2-theta. In particular embodiments, a Form IV hydrobromide salt is characterized by an X-ray powder diffraction pattern which includes the peaks at about 6.45, about 12.96, about 19.38, about 19.79, about 21.37 and about 21.58 degrees 2-theta. In an exemplary embodiment, a Form IV hydrobromide salt is characterized by substantially all of the peaks in its X-ray powder diffraction pattern selected from those at about

°2-Theta

5.99
6.45
7.23
7.84
9.31
9.71
11.07
12.96
13.42
16.34
16.55
17.00
17.93
18.99
19.38
19.79
21.37
21.58
22.65
23.23
24.10
24.82
26.539
27.12
27.89
28.43
28.74

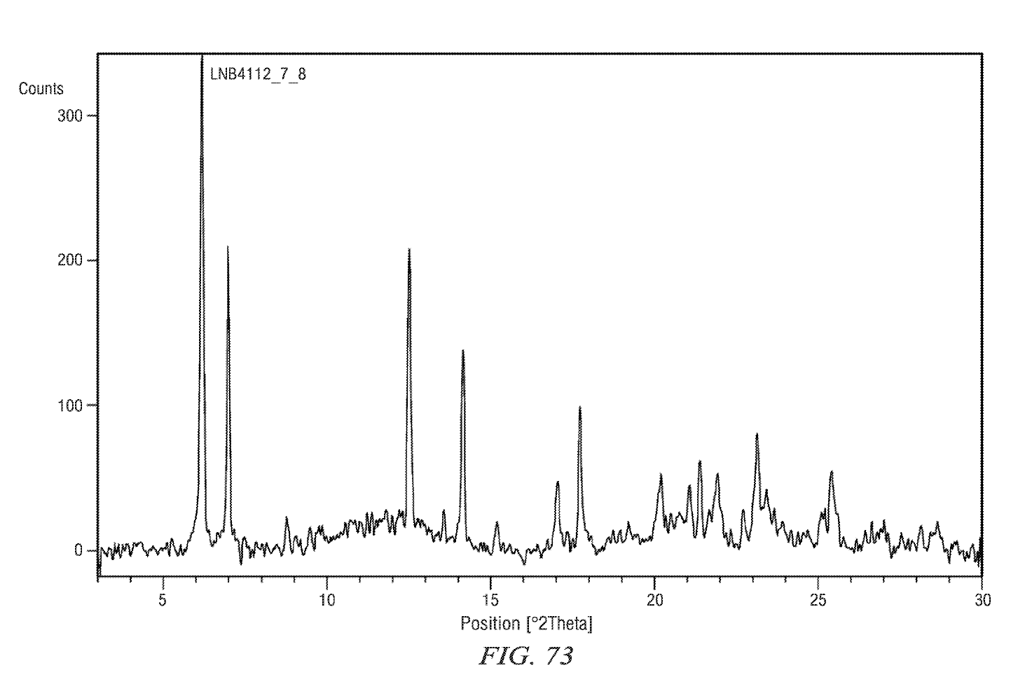

In some embodiments, compound 2 is a Form V hydrobromide salt. In some such embodiments, a Form V hydrobromide salt is a N,N-dimethylformamide (DMF) solvate. In some embodiments, a Form V hydrobromide salt is characterized by a powder X-ray diffraction pattern substantially similar to that depicted in FIG. 73. According to one embodiment, a Form V hydrobromide salt is characterized by one or more peaks in its powder X-ray diffraction pattern selected from those at about 6.17, about 6.99, about 12.50, about 14.14, about 17.72 and about 23.12 degrees 2-theta. In some embodiments, a Form V hydrobromide salt is characterized by two or more peaks in its powder X-ray diffraction pattern selected from those at about 6.17, about 6.99, about 12.50, about 14.14, about 17.72 and about 23.12 degrees 2-theta. In certain embodiments, a Form V hydrobromide salt is characterized by three or more peaks in its powder X-ray diffraction pattern selected from those at about 6.17, about 6.99, about 12.50, about 14.14, about 17.72 and about 23.12 degrees 2-theta. In some embodiments, a Form V hydrobromide salt is characterized by four or more peaks in its powder X-ray diffraction pattern selected from those at about 6.17, about 6.99, about 12.50, about 14.14, about 17.72 and about 23.12 degrees 2-theta. In some embodiments, a Form V hydrobromide salt is characterized by five or more peaks in its powder X-ray diffraction pattern selected from those at about 6.17, about 6.99, about 12.50, about 14.14, about 17.72 and about 23.12 degrees 2-theta. In particular embodiments, a Form V hydrobromide salt is characterized by an X-ray powder diffraction pattern which includes the peaks at about 6.17, about 6.99, about 12.50, about 14.14, about 17.72 and about 23.12 degrees 2-theta. In an exemplary embodiment, a Form V hydrobromide salt is characterized by substantially all of the peaks in its X-ray powder diffraction pattern selected from those at about

°2-Theta

6.17
6.99
8.76
12.50
13.56
14.14
15.19
17.03
17.72
20.20
21.06
21.38
21.66
21.90
22.33
22.70
23.12
23.41
23.66
23.88
24.16
24.57
25.15
25.41
26.64
26.97
28.12
28.42
28.61

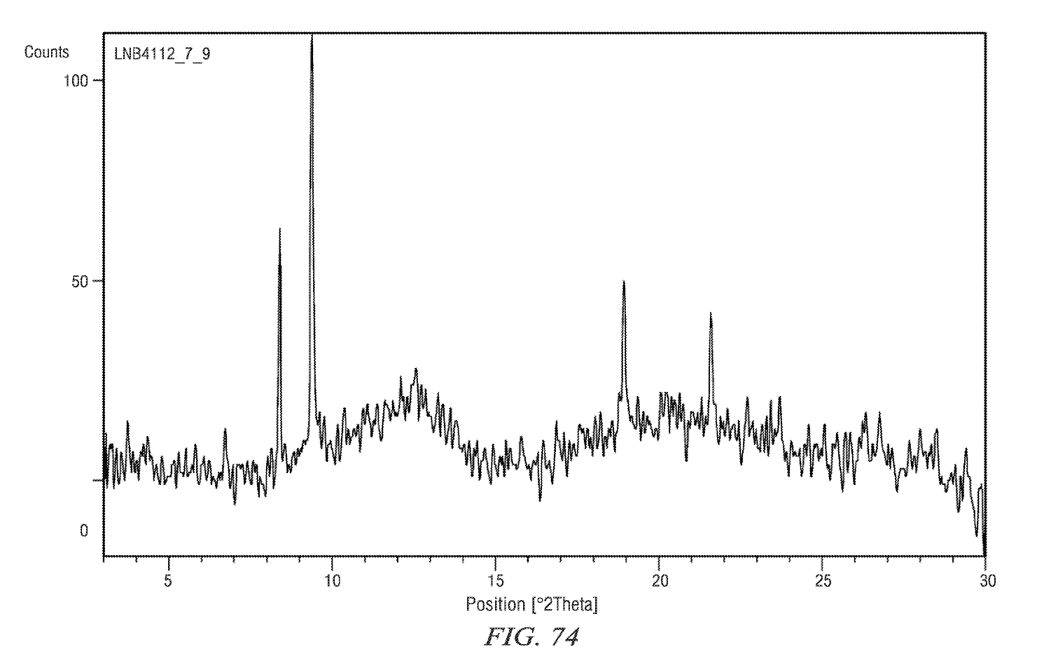

In some embodiments, compound 2 is a Form VI hydrobromide salt. In some such embodiments, a Form VI hydrobromide salt is a dimethylsulfoxide (DMSO) solvate. In some embodiments, a Form VI hydrobromide salt is characterized by a powder X-ray diffraction pattern substantially similar to that depicted in FIG. 74. According to one embodiment, a Form VI hydrobromide salt is characterized by one or more peaks in its powder X-ray diffraction pattern selected from those at about 8.38, about 9.38, about 18.93, and about 21.58 degrees 2-theta. In some embodiments, a Form VI hydrobromide salt is characterized by two or more peaks in its powder X-ray diffraction pattern selected from those at about 8.38, about 9.38, about 18.93, and about 21.58 degrees 2-theta. In certain embodiments, a Form VI hydrobromide salt is characterized by three or more peaks in its powder X-ray diffraction pattern selected from those at about 8.38, about 9.38, about 18.93, and about 21.58 degrees 2-theta. In particular embodiments, a Form VI hydrobromide salt is characterized by an X-ray powder diffraction pattern which includes the peaks at about 8.38, about 9.38, about 18.93, and about 21.58 degrees 2-theta. In an exemplary embodiment, a Form VI hydrobromide salt is characterized by substantially all of the peaks in its X-ray powder diffraction pattern selected from those at about

°2-Theta

8.38
9.38
18.93
21.58

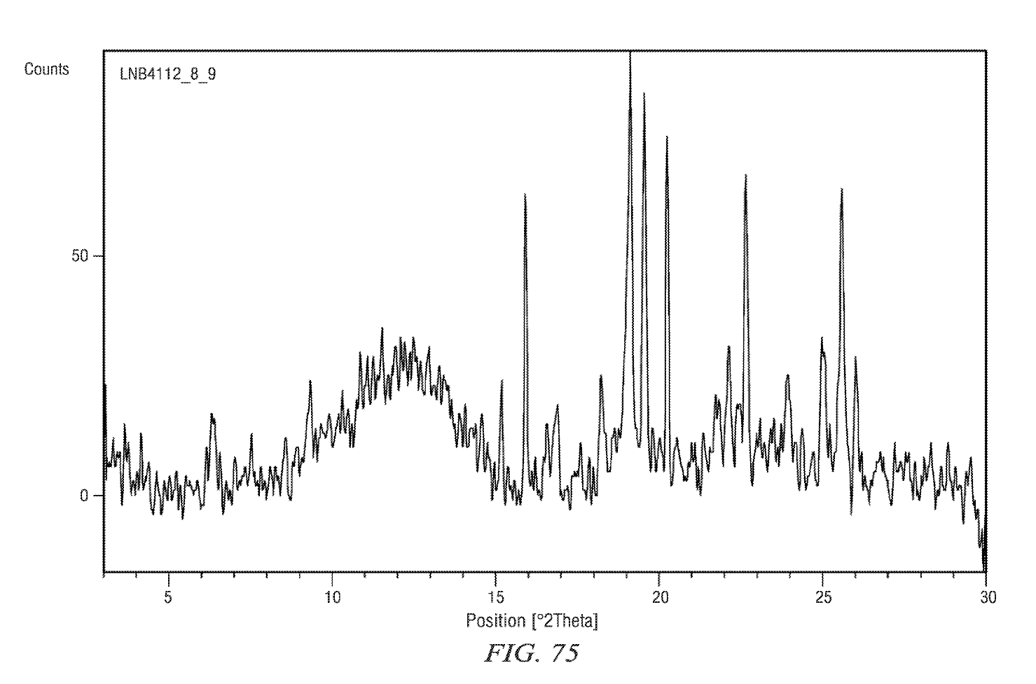

In some embodiments, compound 2 is a Form VII hydrobromide salt. In some such embodiments, a Form VII hydrobromide salt is a dimethylsulfoxide (DMSO) solvate. In some embodiments, a Form VII hydrobromide salt is characterized by a powder X-ray diffraction pattern substantially similar to that depicted in FIG. 75. According to one embodiment, a Form VII hydrobromide salt is characterized by one or more peaks in its powder X-ray diffraction pattern selected from those at about 15.91, about 19.10, about 19.53, about 20.24, about 22.64 and about 25.58 degrees 2-theta. In some embodiments, a Form VII hydrobromide salt is characterized by two or more peaks in its powder X-ray diffraction pattern selected from those at about 15.91, about 19.10, about 19.53, about 20.24, about 22.64 and about 25.58 degrees 2-theta. In certain embodiments, a Form VII hydrobromide salt is characterized by three or more peaks in its powder X-ray diffraction pattern selected from those at about 15.91, about 19.10, about 19.53, about 20.24, about 22.64 and about 25.58 degrees 2-theta. In some embodiments, a Form VII hydrobromide salt is characterized by four or more peaks in its powder X-ray diffraction pattern selected from those at about 15.91, about 19.10, about 19.53, about 20.24, about 22.64 and about 25.58 degrees 2-theta. In some embodiments, a Form VII hydrobromide salt is characterized by five or more peaks in its powder X-ray diffraction pattern selected from those at about 15.91, about 19.10, about 19.53, about 20.24, about 22.64 and about 25.58 degrees 2-theta. In particular embodiments, a Form VII hydrobromide salt is characterized by an X-ray powder diffraction pattern which includes the peaks at about 15.91, about 19.10, about 19.53, about 20.24, about 22.64 and about 25.58 degrees 2-theta. In an exemplary embodiment, a Form VII hydrobromide salt is characterized by substantially all of the peaks in its X-ray powder diffraction pattern selected from those at about

°2-Theta

6.35
8.88
9.29
15.18
15.91
16.54
16.88
17.56
18.22
19.10
19.53
20.24
21.78
22.12
22.64
23.93
24.37
25.00
25.58
26.00

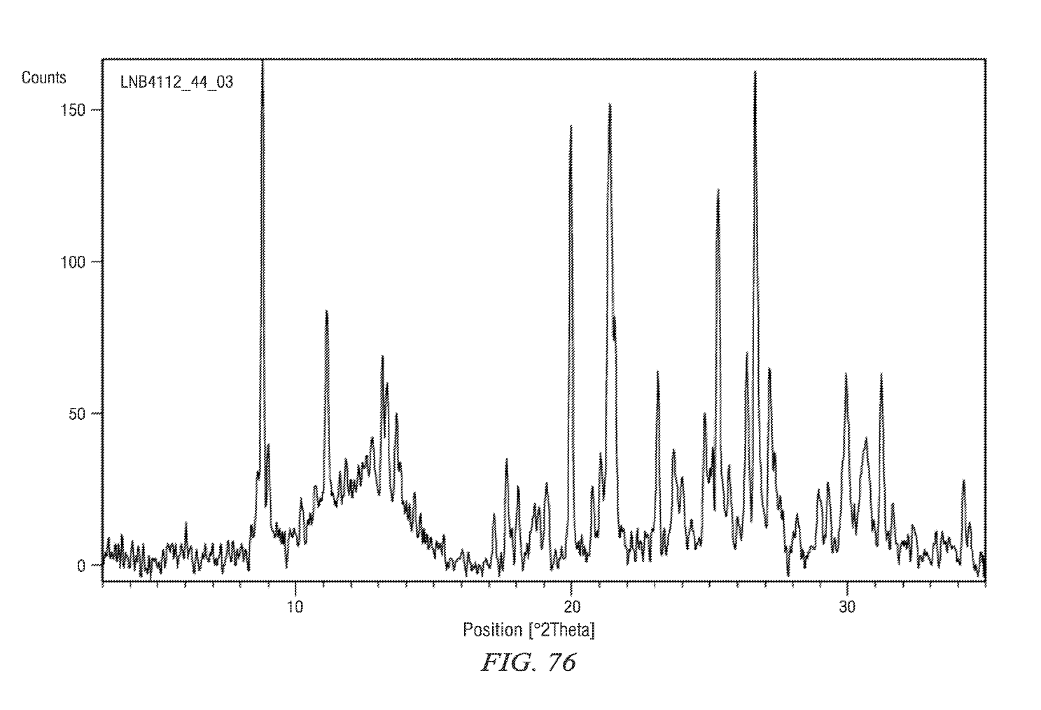

In some embodiments, compound 2 is a Form VIII hydrobromide salt. In some such embodiments, a Form VIII hydrobromide salt is a hydrate. In some embodiments, a Form VIII hydrobromide salt is characterized by a powder X-ray diffraction pattern substantially similar to that depicted in FIG. 76. According to one embodiment, a Form VIII hydrobromide salt is characterized by one or more peaks in its powder X-ray diffraction pattern selected from those at about 8.79, about 11.13, about 19.97, about 21.31, about 21.56, about 25.30 and about 26.65 degrees 2-theta. In some embodiments, a Form VIII hydrobromide salt is characterized by two or more peaks in its powder X-ray diffraction pattern selected from those at about 8.79, about 11.13, about 19.97, about 21.31, about 21.56, about 25.30 and about 26.65 degrees 2-theta. In certain embodiments, a Form VIII hydrobromide salt is characterized by three or more peaks in its powder X-ray diffraction pattern selected from those at about 8.79, about 11.13, about 19.97, about 21.31, about 21.56, about 25.30 and about 26.65 degrees 2-theta. In some embodiments, a Form VIII hydrobromide salt is characterized by four or more peaks in its powder X-ray diffraction pattern selected from those at about 8.79, about 11.13, about 19.97, about 21.31, about 21.56, about 25.30 and about 26.65 degrees 2-theta. In some embodiments, a Form VIII hydrobromide salt is characterized by five or more peaks in its powder X-ray diffraction pattern selected from those at about 8.79, about 11.13, about 19.97, about 21.31, about 21.56, about 25.30 and about 26.65 degrees 2-theta. In some embodiments, a Form VIII hydrobromide salt is characterized by six or more peaks in its powder X-ray diffraction pattern selected from those at about 8.79, about 11.13, about 19.97, about 21.31, about 21.56, about 25.30 and about 26.65 degrees 2-theta. In particular embodiments, a Form VIII hydrobromide salt is characterized by an X-ray powder diffraction pattern which includes the peaks at about 8.79, about 11.13, about 19.97, about 21.31, about 21.56, about 25.30 and about 26.65 degrees 2-theta. In an exemplary embodiment, a Form VIII hydrobromide salt is characterized by substantially all of the peaks in its X-ray powder diffraction pattern selected from those at about

°2-Theta

8.79
9.00
10.22
11.13
13.15
13.30
13.65
17.19
17.65
18.07
18.69
19.09
19.97
20.75
21.05
21.31
21.56
23.12
23.71
24.00
24.82
25.30
25.71
26.34
26.65
27.17
28.18
28.97
29.31
29.96
30.65
31.23
31.64
34.21
34.43

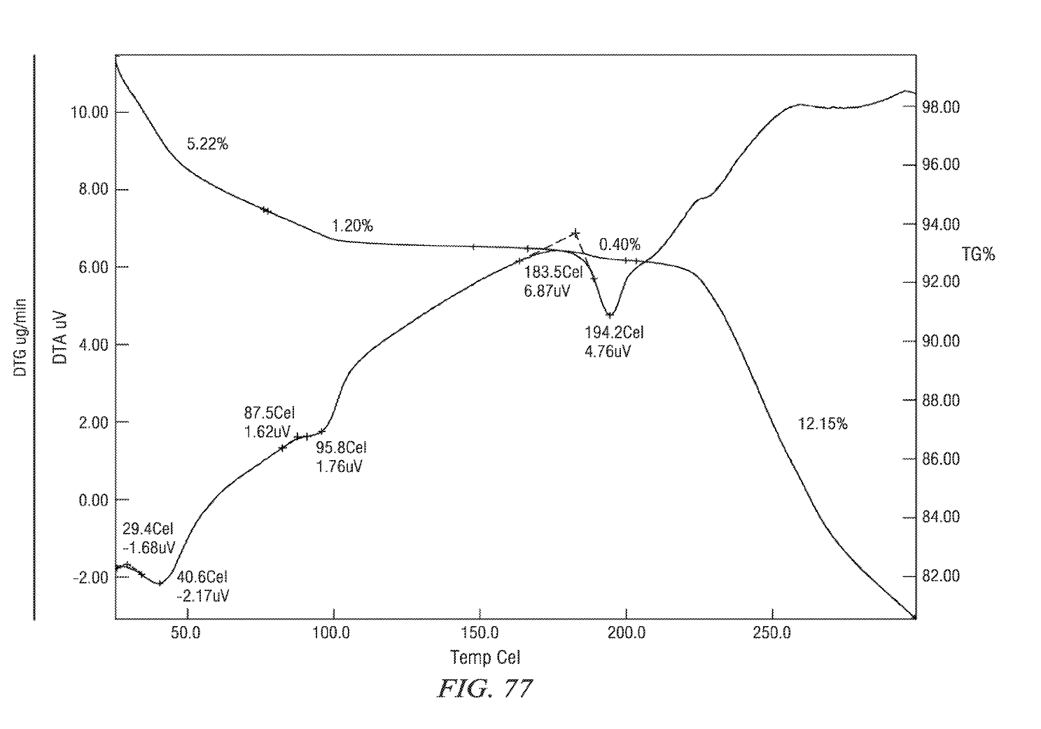

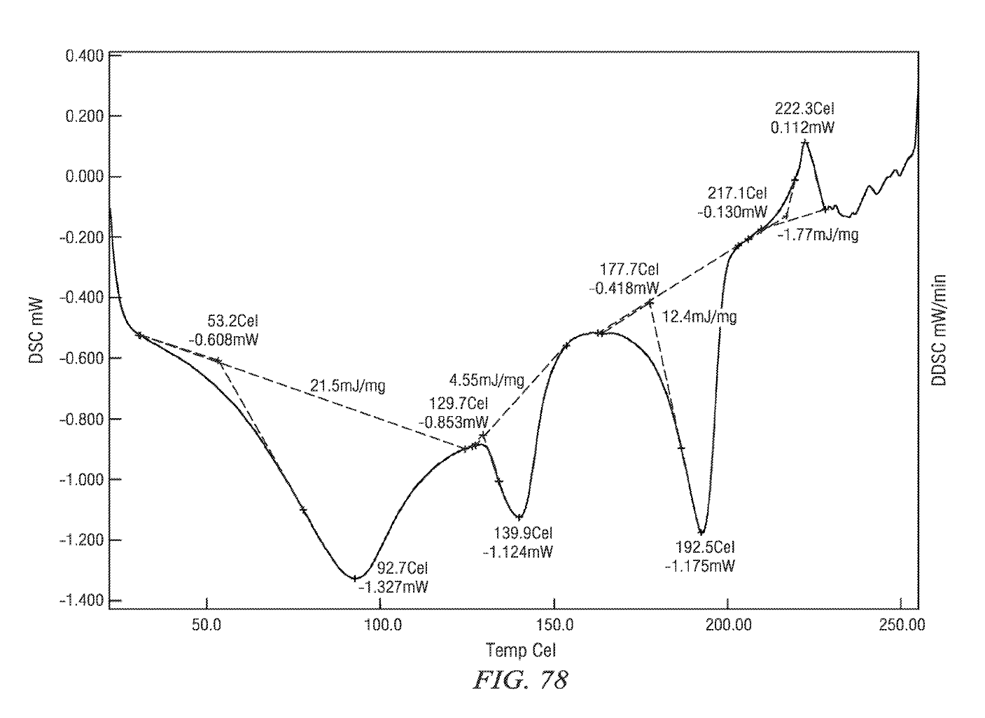

In some embodiments, a Form VIII hydrobromide salt has a thermogravimetric analysis pattern substantially similar to that depicted in FIG. 77. In some embodiments, a Form VIII hydrobromide salt has a differential scanning calorimetry pattern substantially similar to that depicted in FIG. 78. In some embodiments, a Form VIII hydrobromide salt is characterized by substantial similarity to two or more of these figures simultaneously.

In certain embodiments, compound 2 is a hydrochloric acid salt. In some embodiments, compound 2 is a mono-hydrochloric acid salt. In some embodiments, compound 2 is a bis-hydrochloric acid salt.

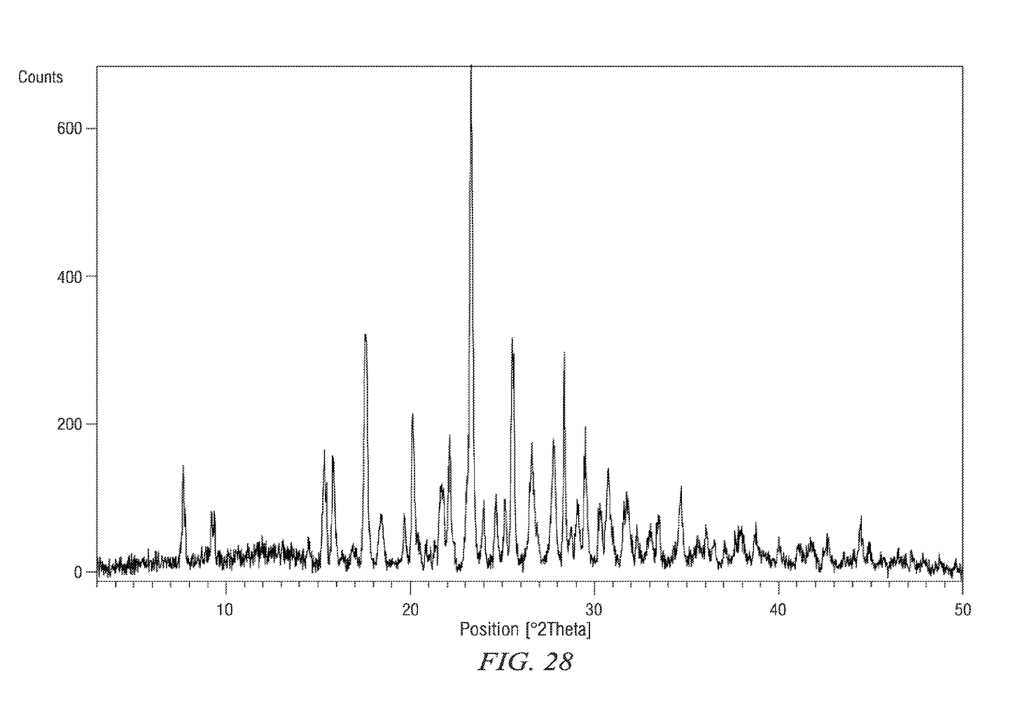

According to one aspect, a bis-hydrochloride salt has a powder X-ray diffraction pattern substantially similar to that depicted in FIG. 28. According to one embodiment, a bis-hydrochloride salt is characterized by one or more peaks in its powder X-ray diffraction pattern selected from those at about 17.58, about 23.32, about 25.53 and about 28.37 degrees 2-theta. In some embodiments, a bis-hydrochloride salt is characterized by two or more peaks in its powder X-ray diffraction pattern selected from those at about 17.58, about 23.32, about 25.53 and about 28.37 degrees 2-theta. In certain embodiments, a bis-hydrochloride salt is characterized by three or more peaks in its powder X-ray diffraction pattern selected from those at about 17.58, about 23.32, about 25.53 and about 28.37 degrees 2-theta. In particular embodiments, a bis-hydrochloride salt is characterized by substantially all of the peaks in its X-ray powder diffraction pattern selected from those at about 17.58, 20.13, 22.14, 23.32, 25.53, 26.60, 27.80 and 28.37 degrees 2-theta. In an exemplary embodiment, a bis-hydrochloride salt is characterized by substantially all of the peaks in its X-ray powder diffraction pattern selected from those at about

°2-Theta

3.04
3.84
5.40
6.34
7.67
9.20
9.37
10.62
14.48
15.34
15.47
15.81
16.34
16.87
17.58
18.44
19.68
20.13
20.86
21.38
21.67
22.14
23.32
23.99
24.63
25.15
25.53
26.60
27.80
28.37
28.73
29.08
29.50
30.21
30.74
31.56
31.79
32.29
33.03
33.46
34.66
35.62
36.07
36.47
37.08
37.91
38.71
40.02
41.01
41.78
42.64
44.43
44.89
45.62
46.46
46.87
47.18
47.88
48.14
48.72
49.63

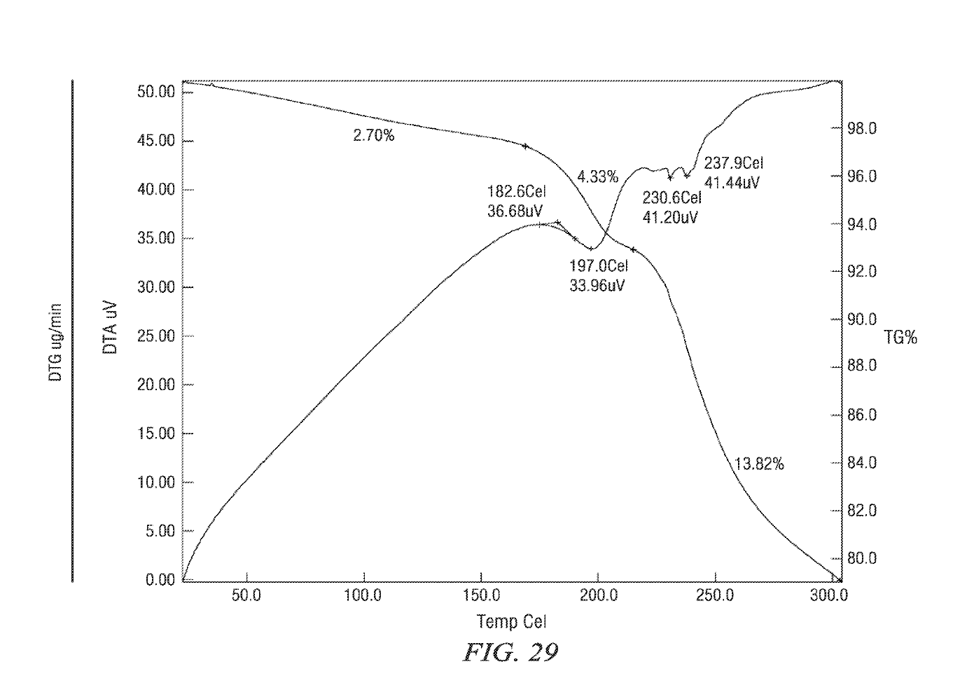

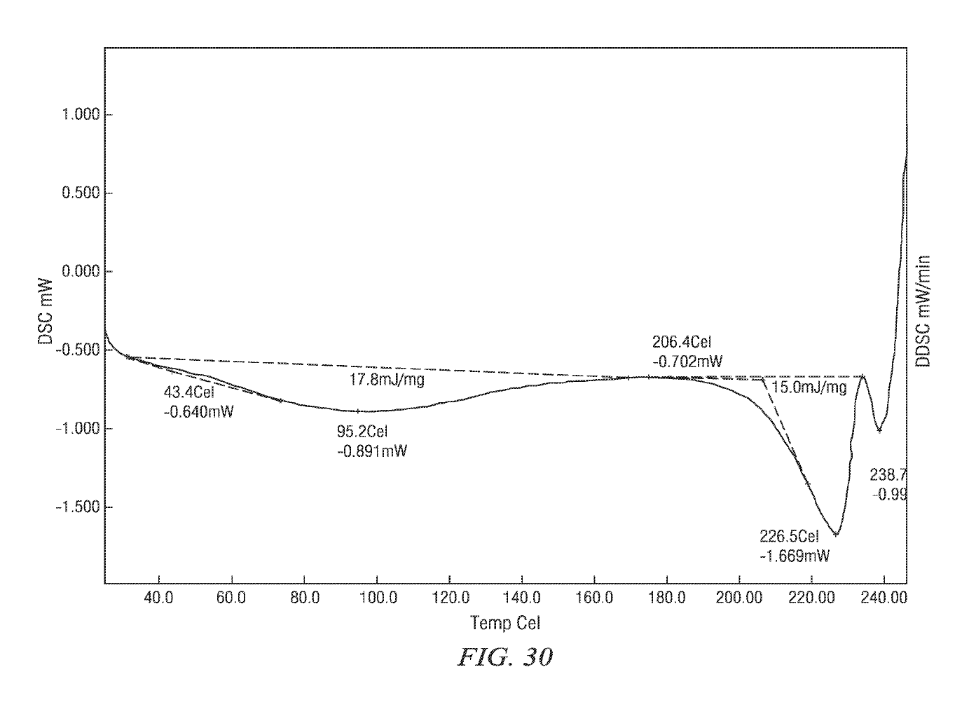

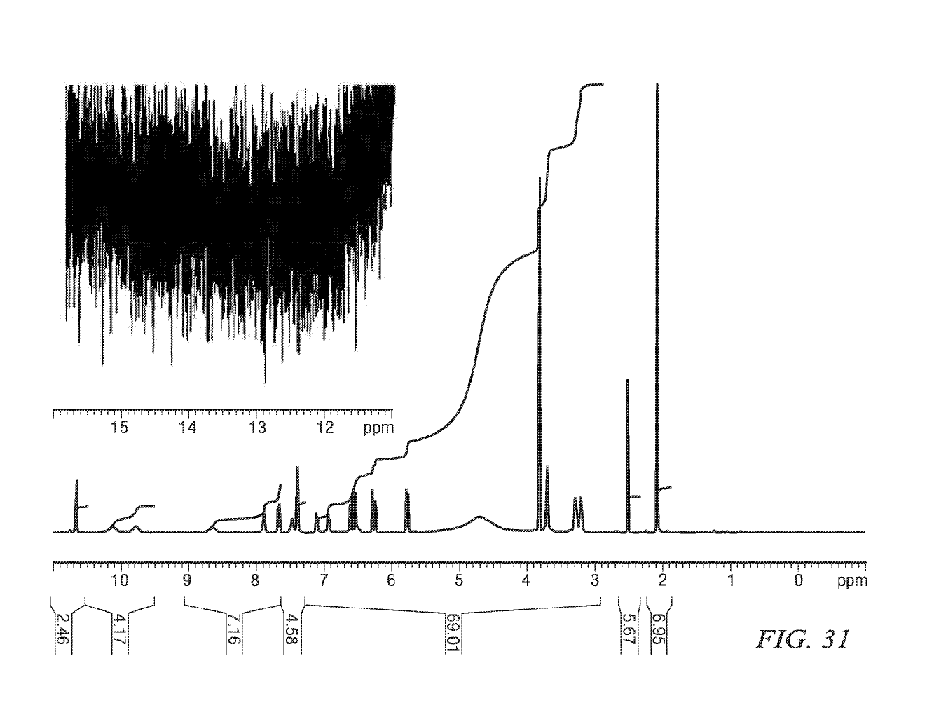

According to another aspect, a bis-hydrochloride salt has a thermogravimetric analysis pattern substantially similar to that depicted in FIG. 29. According to yet another aspect, a bis-hydrochloride salt has a differential scanning calorimetry pattern substantially similar to that depicted in FIG. 30. According to another embodiment, a bis-hydrochloride salt has an ¹H-NMR spectrum substantially similar to that depicted in FIG. 31.

In certain embodiments, compound 2 is a maleic acid salt. In some embodiments, compound 2 is a mono-maleic acid salt. In some embodiments, compound 2 is a bis-maleic acid salt.

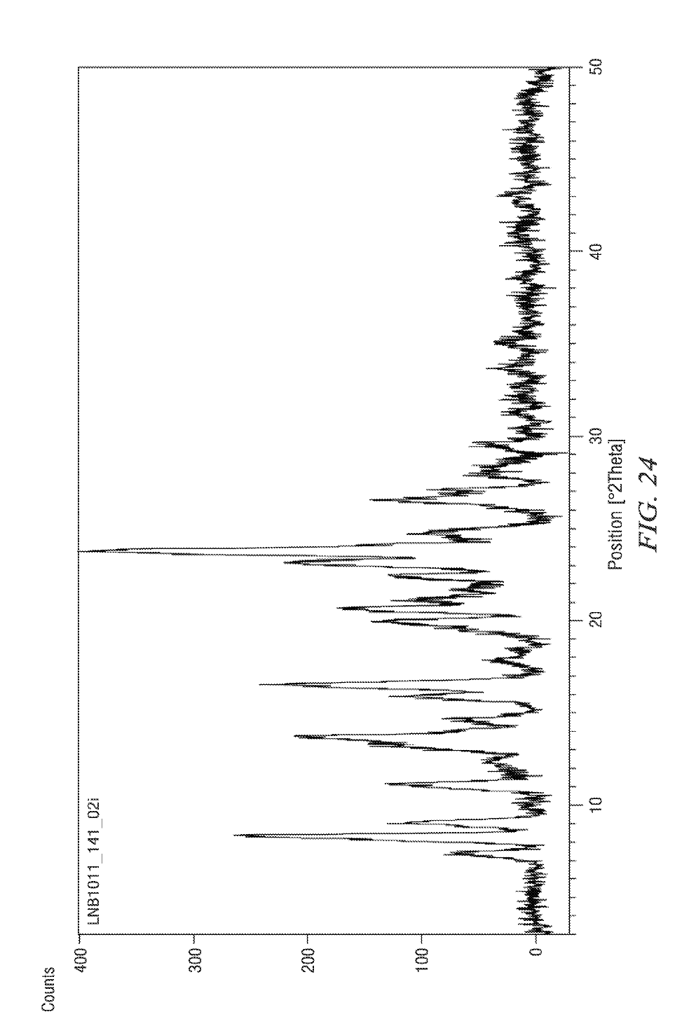

According to one aspect, a mono-maleate salt has a powder X-ray diffraction pattern substantially similar to that depicted in FIG. 24. According to one embodiment, a mono-maleate salt is characterized by one or more peaks in its powder X-ray diffraction pattern selected from those at about 8.38, about 23.59 and about 23.80 degrees 2-theta. In some embodiments, a mono-maleate salt is characterized by two or more peaks in its powder X-ray diffraction pattern selected from those at about 8.38, about 23.59 and about 23.80 degrees 2-theta. In certain embodiments, a mono-maleate salt is characterized by three peaks in its powder X-ray diffraction pattern selected from those at about 8.38, about 23.59 and about 23.80 degrees 2-theta. In particular embodiments, a mono-maleate salt is characterized by substantially all of the peaks in its X-ray powder diffraction pattern selected from those at about 8.38, 13.74, 16.35, 16.54, 20.67, 23.15, 23.59 and 23.80 degrees 2-theta. In an exemplary embodiment, a mono-maleate salt is characterized by substantially all of the peaks in its X-ray powder diffraction pattern selected from those at about

°2-Theta

7.42
8.38
9.06
9.91
10.13
10.45
10.62
11.16
12.40
13.15
13.74
14.65
15.91
16.35
16.54
17.86
19.96
20.67
22.50
23.15
23.59
23.80
24.75
26.52
27.13
27.90
29.53
30.37
31.30
32.04
33.68
35.05
38.51
41.05
43.03
45.85
46.06
46.44
46.69
48.23

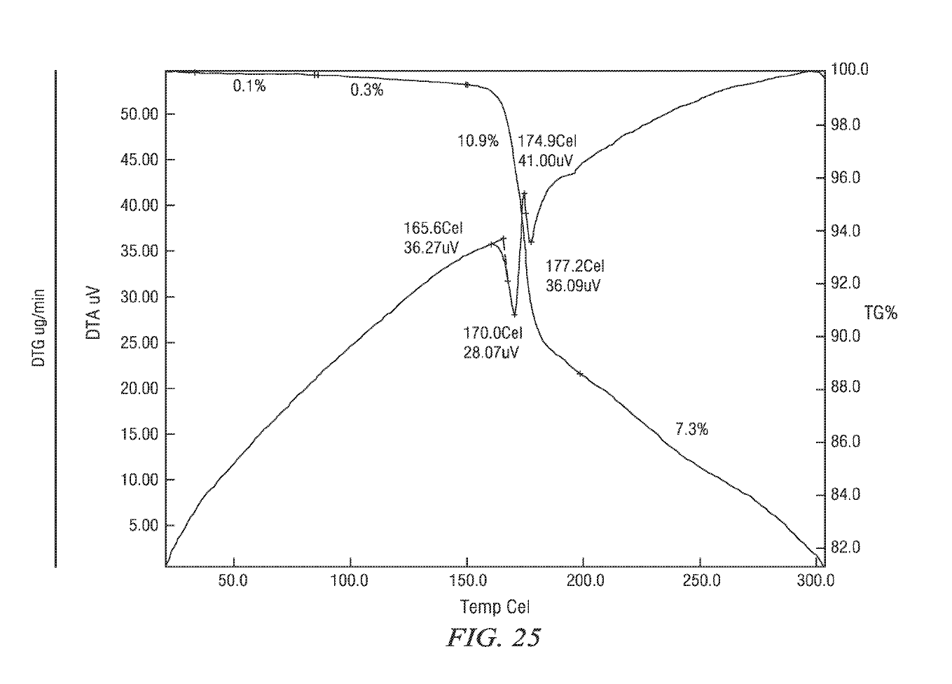

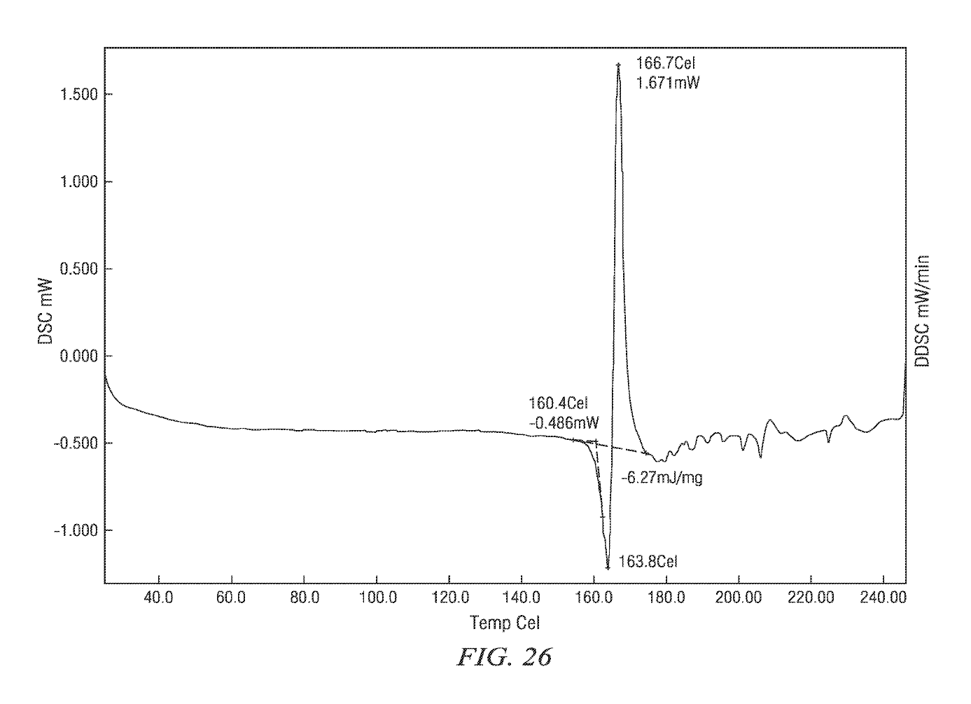

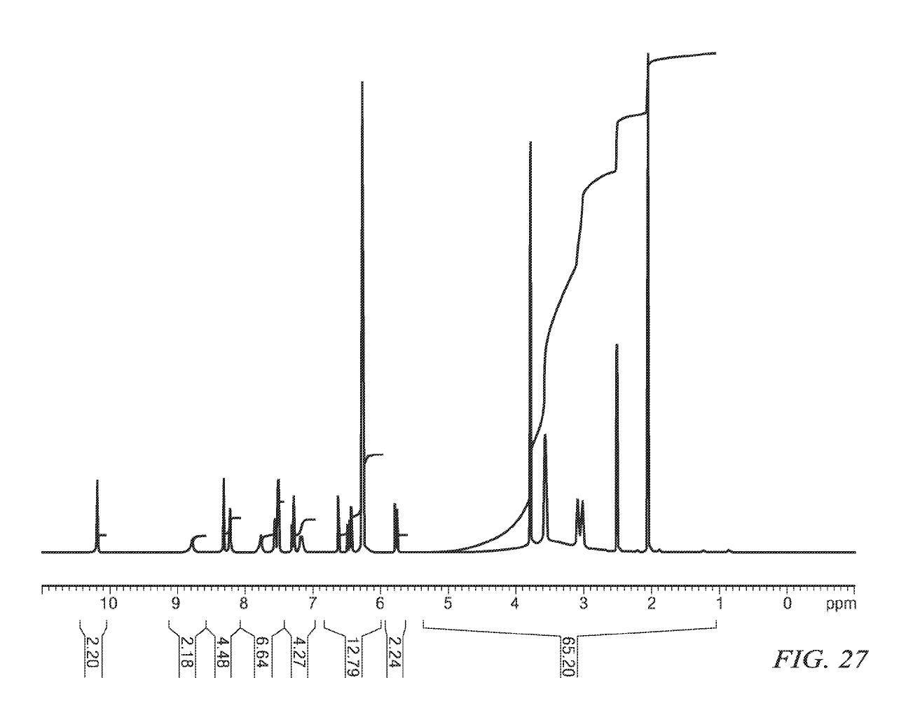

According to another aspect, a mono-maleate salt has a thermogravimetric analysis pattern substantially similar to that depicted in FIG. 25. According to yet another aspect, a mono-maleate salt has a differential scanning calorimetry pattern substantially similar to that depicted in FIG. 26. According to another embodiment, a mono-maleate salt has an ¹H-NMR spectrum substantially similar to that depicted in FIG. 27.

It will be appreciated that any of the above-described polymorph forms can be characterized, for example, by reference to any of the peaks in their respective X-ray diffraction patterns. Accordingly, in some embodiments, a polymorph described herein is characterized by one, two, three, four, five, six, seven, eight, nine, ten, eleven, twelve, thirteen, fourteen, fifteen, sixteen, seventeen, eighteen, nineteen, twenty or more XRPD peaks (° 2θ).

In certain embodiments, compound 2 is a methanesulfonic acid salt. In some embodiments, compound 2 is a mono-methansulfonic acid salt. In some embodiments, compound 2 is a bis-methanesulfonic acid salt.

In certain embodiments, compound 2 is a naphthalene-2-sulfonic acid salt. In some embodiments, compound 2 is a mono-naphthalene-2-sulfonic acid salt. In some embodiments, compound 2 is a bis-naphthalene-2-sulfonic acid salt.

In certain embodiments, compound 2 is a 1,5-naphthalene disulfonic acid salt. In some embodiments, compound 2 is a mono-1,5-naphthalene disulfonic acid salt. In some embodiments, compound 2 is a bis-1,5-naphthalene disulfonic acid salt.

In certain embodiments, compound 2 is an oxalic acid salt. In some embodiments, compound 2 is a mono-oxalic acid salt. In some embodiments, compound 2 is a bis-oxalic acid salt.

In certain embodiments, compound 2 is a p-toluenesulfonic acid (tosylate) salt. In some embodiments, compound 2 is a mono-p-toluenesulfonic acid salt. In some embodiments, compound 2 is a bis-p-toluenesulfonic acid salt.

In certain embodiments, compound 2 is a 2,4,6-trihydroxybenzoic acid salt. In some embodiments, compound 2 is a mono-2,4,6-trihydroxybenzoic acid salt. In some embodiments, compound 2 is a bis-2,4,6-trihydroxybenzoic acid salt.

According to another embodiment, the present invention provides compound 2 as an amorphous solid. Amorphous solids are well known to one of ordinary skill in the art and are typically prepared by such methods as lyophilization, melting, and precipitation from supercritical fluid, among others.

### General Methods of Providing Compound 2:

Compound 1 is prepared according to the methods described in detail in the '061 application, the entirety of which is hereby incorporated herein by reference. Compound 2 is prepared from Compound 1, according to the Scheme below.

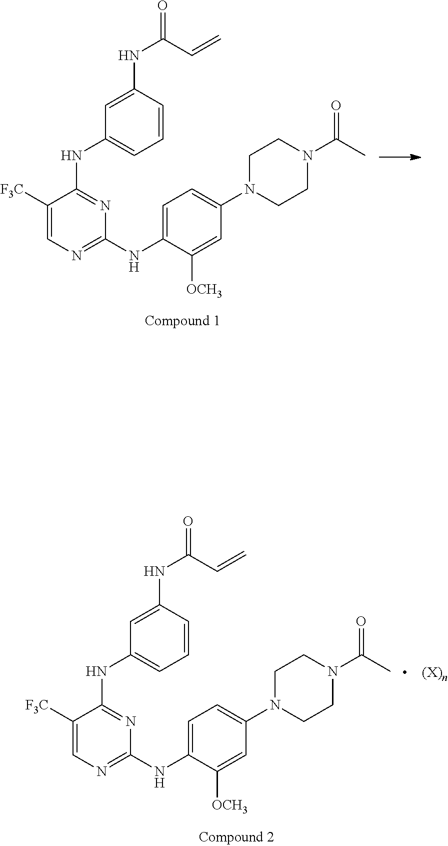 [SMILES: C=CC(=O)Nc1cccc(Nc2nc(Nc3ccc(N4CCN(C(C)=O)CC4)cc3OC)ncc2C(F)(F)F)c1 ; C=CC(=O)Nc1cccc(Nc2nc(Nc3ccc(N4CCN(C(C)=O)CC4)cc3OC)ncc2C(F)(F)F)c1]

As depicted in the general Scheme above, Compound 2 is prepared from Compound 1 by combining Compound 1 with either one or two equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid to form the salt thereof. Thus, another aspect of the present invention provides a method for preparing Compound 2:

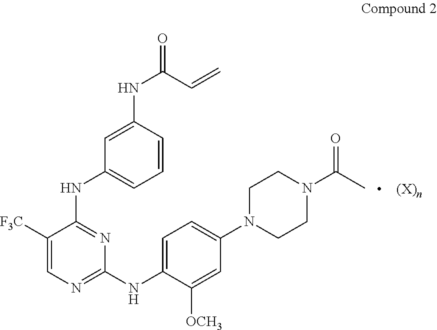 [SMILES: C=CC(=O)Nc1cccc(Nc2nc(Nc3ccc(N4CCN(C(C)=O)CC4)cc3OC)ncc2C(F)(F)F)c1]

comprising the steps of:

providing Compound 1:

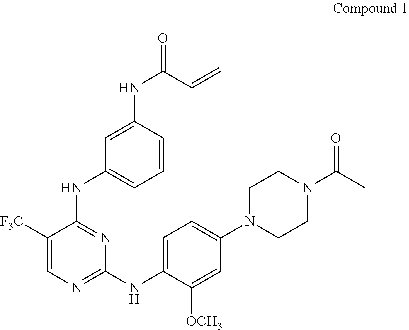 [SMILES: C=CC(=O)Nc1cccc(Nc2nc(Nc3ccc(N4CCN(C(C)=O)CC4)cc3OC)ncc2C(F)(F)F)c1]

combining Compound 1 with one or two equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid in a suitable solvent; and optionally isolating Compound 2.

A suitable solvent may solubilize one or more of the reaction components, or, alternatively, the suitable solvent may facilitate the agitation of a suspension of one or more of the reaction components. Examples of suitable solvents useful in the present invention are a protic solvent, a polar aprotic solvent, a nonpolar solvent or mixtures thereof. In certain embodiments, suitable solvents include water, an ether, an ester, an alcohol, a halogenated solvent, a ketone, or a mixture thereof. In certain embodiments, the suitable solvent is methanol, ethanol, isopropanol, ethyl acetate, isopropyl acetate, methyl ethyl ketone, methyl isobutyl ketone or acetone. In certain embodiments, the suitable solvent is dichloromethane. In other embodiments, suitable solvents include tetrahydrofuran, dimethylformamide, dimethylsulfoxide, glyme, diglyme, methyl t-butyl ether, t-butanol, n-butanol, and acetonitrile. In some embodiments, the suitable solvent is cyclohexane.

According to another embodiment, the present invention provides a method for preparing Compound 2:

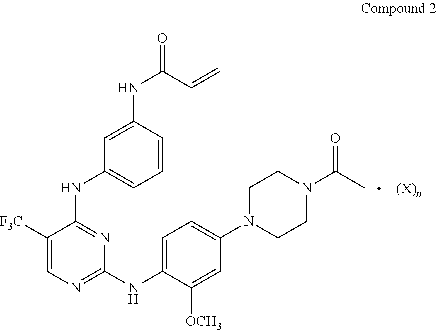 [SMILES: C=CC(=O)Nc1cccc(Nc2nc(Nc3ccc(N4CCN(C(C)=O)CC4)cc3OC)ncc2C(F)(F)F)c1]

comprising the steps of:

combining Compound 1:

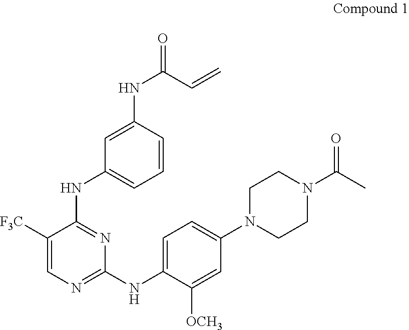 [SMILES: C=CC(=O)Nc1cccc(Nc2nc(Nc3ccc(N4CCN(C(C)=O)CC4)cc3OC)ncc2C(F)(F)F)c1]

with a suitable solvent and optionally heating to form a solution thereof;

adding one or two equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid to said solution; and

optionally isolating Compound 2.

As described generally above, Compound 1 is dissolved or suspended in a suitable solvent, optionally with heating. In certain embodiments Compound 1 is dissolved at about 20 to about 60° C. In other embodiments, Compound 1 is dissolved at about 20 to about 25° C., such as about ambient temperature. In still other embodiments, compound 1 is dissolved at the boiling temperature of the solvent. In other embodiments, compound 1 is dissolved without heating.

In certain embodiments, about 1 equivalent of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid is added to Compound 1 to afford Compound 2. In other embodiments, about 2 equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid is added to Compound 1 to afford Compound 2. In yet other embodiments, greater than 2 equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid is added to Compound 1 to afford Compound 2. In still other embodiments, about 0.9 to about 1.1 equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid is added to Compound 1 to afford Compound 2. In another embodiment, about 0.99 to about 1.01 equivalents of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid is added to Compound 1 to afford Compound 2. In further embodiments, about 1.8 to about 2.2 equivalents, such as about 1.98 to 2.02 equivalents, of benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid is added to Compound 1 to afford Compound 2.

It will be appreciated that the acid may be added to the mixture of Compound 1 and a suitable solvent in any suitable form. For example, the acid may be added in solid form or as a solution or a suspension in a suitable solvent. The suitable solvent may be the same suitable solvent as that which is combined with Compound 1 or may be a different solvent. According to one embodiment, the acid is added in solid form. In certain embodiments, the acid is combined with a suitable solvent prior to adding to Compound 1. According to another embodiment, the acid is added as a solution in a suitable solvent. In certain embodiments, suitable solvents include water, an ether, an ester, an alcohol, a halogenated solvent, a ketone, or a mixture thereof. In certain embodiments, the suitable solvent is methanol, ethanol, isopropanol, ethyl acetate, isopropyl acetate, methyl ethyl ketone, methyl isobutyl ketone or acetone. In certain embodiments, the suitable solvent is dichloromethane. In other embodiments, suitable solvents include tetrahydrofuran, dimethylformamide, dimethylsulfoxide, glyme, diglyme, methyl t-butyl ether, t-butanol, n-butanol, and acetonitrile. In some embodiments, the suitable solvent is cyclohexane. In certain embodiments the suitable solvent is selected from those above and is anhydrous.

In certain embodiments, the resulting mixture containing Compound 2 is cooled. In other embodiments, the mixture containing Compound 2 is cooled below 20° C., such as below 10° C.

In certain embodiments, Compound 2 precipitates from the mixture. In another embodiment, Compound 2 crystallizes from the mixture. In other embodiments, Compound 2 crystallizes from solution following seeding of the solution (i.e., adding crystals of Compound 2 to the solution).

Crystalline Compound 2 can precipitate out of the reaction mixture, or be generated by removal of part or all of the solvent through methods such as evaporation, distillation, filtration (e.g., nanofiltration, ultrafiltration), reverse osmosis, absorption and reaction, by adding an anti-solvent such as water, MTBE or heptane, by cooling or by different combinations of these methods.

As described generally above, Compound 2 is optionally isolated. It will be appreciated that Compound 2 may be isolated by any suitable physical means known to one of ordinary skill in the art. In certain embodiments, precipitated solid compound 2 is separated from the supernatant by filtration. In other embodiments, precipitated solid Compound 2 is separated from the supernatant by decanting the supernatant.

In certain embodiments, precipitated solid Compound 2 is separated from the supernatant by filtration.

In certain embodiments, isolated Compound 2 is dried in air. In other embodiments isolated Compound 2 is dried under reduced pressure, optionally at elevated temperature.

### Uses, Formulation and Administration Pharmaceutically Acceptable Compositions

According to another embodiment, the invention provides a composition comprising Compound 2 and a pharmaceutically acceptable carrier, adjuvant, or vehicle. The amount of Compound 2 in compositions of this invention is such that it is effective to measurably inhibit a protein kinase, particularly an EGFR kinase, or a mutant thereof, in a biological sample or in a patient. In certain embodiments, a composition of this invention is formulated for administration to a patient in need of such composition. In some embodiments, a composition of this invention is formulated for oral administration to a patient.

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, Vitamin E polyethylene glycol succinate (d-alpha tocopheryl polyethylene glycol 1000 succinate), sodium carboxymethylcellulose, polyacrylates, waxes, polyethylene-polyoxypropylene-block polymers, gelatine, polyvinyl pyrrolidone vinyl acetate, hydroxypropyl methyl cellulose, madnesium stearate, steric acid, citric acid, mannitol, and wool fat.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be an aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous and non-aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is typically combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of Compound 2 include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, pharmaceutically acceptable compositions of this invention are formulated for oral administration.

The amount of Compound 2 that may be combined with the carrier materials to produce a composition in a single dosage form will vary depending upon the host treated, the particular mode of administration. In certain embodiments, provided compositions are formulated so that a dosage of between 0.01-100 mg/kg body weight/day of Compound 2 can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### Uses of Compounds and Pharmaceutically Acceptable Compositions

Compound 2 and compositions described herein are generally useful for the inhibition of protein kinase activity of one or more enzymes. Examples of kinases that are inhibited by Compound 2 and compositions described herein and against which the methods described herein are useful include EGFR kinase or a mutant thereof. It has been found that Compound 2 is a selective inhibitor of at least one mutation of EGFR, as compared to wild-type (“WT”) EGFR. In certain embodiments, an at least one mutation of EGFR is T790M. In certain embodiments, the at least one mutation of EGFR is a deletion mutation. In some embodiments, the at least one mutation of EGFR is an activating mutation. In certain embodiments, Compound 2 selectively inhibits at least one resistant mutation and at least one activating mutation as compared to WT EGFR. In some embodiments, Compound 2 selectively inhibits at least one deletion mutation and/or at least one point mutation, and is sparing as to WT EGFR inhibition.

A mutation of EGFR can be selected from T790M (resistant or oncogenic), L858R (activating), delE746-A750 (activating), G719S (activating), or a combination thereof.

As used herein, the term “selectively inhibits,” as used in comparison to inhibition of WT EGFR, means that Compound 2 inhibits at least one mutation of EGFR (i.e., at least one deletion mutation, at least one activating mutation, at least one restistant mutation, or a combination of at least one deletion mutation and at least one point mutation) in at least one assay described herein (e.g., biochemical or cellular). In some embodiments, the term “selectively inhibits,” as used in comparison to WT EGFR inhibition means that Compound 2 is at least 50 times more potent, at least 45 times, at least 40, at least 35, at least 30, at least 25, or at least 20 times more potent as an inhibitor of at least one mutation of EGFR, as defined and described herein, as compared to WT EGFR.

As used herein, the term “sparing as to WT EGFR” means that a selective inhibitor of at least one mutation of EGFR, as defined and described above and herein, inhibits EGFR at the upper limit of detection of at least one assay, such as those described in the '061 application (e.g., biochemical or cellular as described in detail in Examples 56-58). In vitro assays include assays that determine inhibition of the phosphorylation activity and/or the subsequent functional consequences, or ATPase activity of activated EGFR (WT or mutant). Alternate in vitro assays quantitate the ability of the inhibitor to bind to EGFR (WT or mutant). Inhibitor binding may be measured by radiolabeling the inhibitor prior to binding, isolating the inhibitor/EGFR (WT or mutant) complex and determining the amount of radiolabel bound. Alternatively, inhibitor binding may be determined by running a competition experiment where new inhibitors are incubated with EGFR (WT or mutant) bound to known radioligands. In some embodiments, the term “sparing as to WT EGFR” means that Compound 2 inhibits WT EGFR with an IC₅₀ of at least 10 μM, at least 9 μM, at least 8 μM, at least 7 μM, at least 6 μM, at least 5 μM, at least 3 μM, at least 2 μM, or at least 1 μM.

In certain embodiments, Compound 2 selectively inhibits (a) at least one activating mutation; and (b) T790M; and (c) is sparing as to WT. In some embodiments, an at least one activating mutation is a deletion mutation. In some embodiments, an at least one activating mutation is a point mutation. In some embodiments, an activating mutation is delE746-A750. In some embodiments, an activating mutation is L858R. In some embodiments, an activating mutation is G719S.

In some embodiments, the at least one mutation of EGFR is L858R and/or T790M.

Without wishing to be bound by any particular theory, it is believed that administration of Compound 2 to a patient having at least one activating mutation may preempt formation of the T790M resistance mutation. Thus, in certain embodiments, the present invention provides a method for inhibiting an activating mutation in a patient comprising administering to the patient Compound 2 or composition thereof, as described herein.

One of ordinary skill in the art will appreciate that certain patients have an oncogenic form of the T790M mutation, i.e., the T790M mutation is present prior to administrating any EGFR kinase inhibitor to a patient and is therefore oncogenic. Accordingly, in some embodiments, the present invention provides a method for inhibiting oncogenic T790M in a patient comprising administering to the patient a provided compound or composition thereof, as described herein.

In certain embodiments, the amount of Compound 2 in a composition is effective to measurably inhibit at least one mutant of EGFR selectively as compared to WT EGFR and other protein kinases (e.g., ErbB2, ErbB4, a TEC-kinase, and/or JAK3), in a biological sample or in a patient.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Compound 2 is an inhibitor of at least one mutant of EGFR and is therefore useful for treating one or more disorders associated with activity of one of more EGFR mutants (e.g., a deletion mutation, an activating mutation, a resistant mutation, or combination thereof). Thus, in certain embodiments, the present invention provides a method for treating a mutant EGFR-mediated disorder comprising the step of administering to a patient in need thereof Compound 2 or pharmaceutically acceptable composition thereof.

As used herein, the term “mutant EGFR-mediated” disorders or conditions as used herein means any disease or other deleterious condition in which at least one mutant of EGFR is known to play a role. In certain embodiments, an at least one mutant of EGFR is T790M. In some embodiments, the at least one mutant of EGFR is a deletion mutation. In certain embodiments, the at least one mutant of EGFR is an activating mutation. In some embodiments, the at least one mutant of EGFR is L858R and/or T790M. In certain embodiments, a provided compound selectively inhibits (a) at least one activating mutation, (b) T790M, and (c) is sparing as to WT. In some embodiments, an at least one activating mutation is a deletion mutation. In some embodiments, an at least one activating mutation is a point mutation. In some embodiments, an activating mutation is delE746-A750. In some embodiments, an activating mutation is L858R. In some embodiments, an activating mutation is G719S.

Accordingly, another embodiment of the present invention relates to treating or lessening the severity of one or more diseases in which at least one mutant of EGFR is known to play a role. Specifically, the present invention relates to a method of treating or lessening the severity of a disease or condition selected from a proliferative disorder, wherein said method comprises administering to a patient in need thereof a compound or composition according to the present invention.

In some embodiments, the present invention provides a method for treating or lessening the severity of one or more disorders selected from a cancer. In some embodiments, the cancer is associated with a solid tumor. In certain embodiments, the cancer is breast cancer, glioblastoma, lung cancer, cancer of the head and neck, colorectal cancer, bladder cancer, or non-small cell lung cancer. In some embodiments, the present invention provides a method for treating or lessening the severity of one or more disorders selected from squamous cell carcinoma, salivary gland carcinoma, ovarian carcinoma, or pancreatic cancer.

In certain embodiments, the present invention provides a method for treating or lessening the severity of neurofibromatosis type I (NF1), neurofibromatosis type II (NF2) Schwann cell neoplasms (e.g. MPNST's), or Schwannomas.

Compound 2 and compositions thereof, according to the method of the present invention, may be administered using any amount and any route of administration effective for treating or lessening the severity of a cancer. The exact amount required will vary from subject to subject, depending on the species, age, and general condition of the subject, the severity of the infection, the particular agent, its mode of administration, and the like. Compound 2 is preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, Compound 2 may be administered orally or parenterally at dosage levels of about 0.01 mg/kg to about 60 mg/kg, or about 0.1 mg/kg to about 50 mg/kg, or about 0.25 mg/kg to about 45 mg/kg and preferably from about 0.5 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to Compound 2, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, polyethylene glycol (e.g., PEG 200, PEG 400, PEG 1000, PEG 2000), propylene glycol, 1,3-butylene glycol, dimethylformamide, oils (in particular, cottonseed, groundnut, corn, germ, olive, castor, and sesame oils), glycerol, tetrahydrofurfuryl alcohol, Vitamin E polyethylene glycol succinate (d-alpha tocopheryl polyethylene glycol 1000 succinate), polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. The liquid forms above can also be filled into a soft or hard capsule to form a solid dosage form. Suitable capsules can be formed from, for example, gelatin, starch and cellulose derivatives (e.g., hydroxycellulose, hydropropylmethylcellulose).

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of Compound 2 of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing Compound 2 of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, Compound 2 is mixed with at least one inert, pharmaceutically acceptable excipient or carrier such as sodium citrate, Avicel, hydroxypropyl cellulose or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, PVP vinyl acetate, and acacia, c) humectants such as glycerol, d) disintegrating agents such as agar-agar, calcium carbonate, potato or tapioca starch, alginic acid, certain silicates, sodium croscarmellose and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, i) lubricants such as talc, calcium stearate, magnesium stearate, solid polyethylene glycols, sodium lauryl sulfate, j) solubilising agents such as Vitamin E polyethylene glycol succinate (d-alpha tocopheryl polyethylene glycol 1000 succinate), steric acid, and mixtures thereof. In the case of capsules, tablets and pills, the dosage form may also comprise buffering agents.

Solid compositions of a similar type may also be employed as fillers in soft and hard-filled capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled capsules using such excipients as lactose or milk sugar as well as high molecular weight polyethylene glycols and the like.

Compound 2 can also be in micro-encapsulated form with one or more excipients as noted above. The solid dosage forms of tablets, dragees, capsules, pills, and granules can be prepared with coatings and shells such as cosmetic coatings, enteric coatings, release controlling coatings and other coatings well known in the pharmaceutical formulating art. In such solid dosage forms the active compound may be admixed with at least one inert diluent such as a polymer, sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of Compound 2 include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutical compositions for use in the present invention may be prepared as a unit dosage form. A person of ordinary skill will appreciate that the unit dosage forms described herein refer to an amount of Compound 2 as a free base (i.e., Compound 1). A person skilled in the art will further appreciate that, when a pharmaceutical composition comprises Compound 2, for example Compound 2 monohydrobromide, the amount of Compound 2 hydrobromide present in the composition is an amount that is equivalent to a unit dose of the free base (i.e., Compound 1). For example, a pharmaceutical composition comprising 28.64 mg of Compound 2 monohydrobromide would provide a 25 mg unit dose of the free base (i.e., Compound 1).

In some embodiments, a pharmaceutically acceptable composition comprises a unit dose of Compound 2. In some embodiments, the unit dose of Compound 2 is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg or about 1000 mg.

In some embodiments, Compound 2, or a pharmaceutically acceptable composition thereof, is administered once, twice, three, or four times a day. In some embodiments, Compound 2, or a pharmaceutically acceptable composition thereof, is administered once daily (“QD”). In some embodiments, Compound 2, or a pharmaceutically acceptable composition thereof, is administered twice daily. In some embodiments, twice daily administration refers to a compound or composition that is administered “BID”. A “BID” dose is a particular dose (e.g., a 125 mg dose) that is administered twice a day (i.e., two doses of 125 mg administered at two different times in one day). In some embodiments, twice daily administration refers to a compound or composition that is administered in two different doses, wherein the first administered dose differs from the second administered dose. For example, a 250 mg dose administered twice daily can be administered as two separate doses, one 150 mg dose and one 100 mg dose, wherein each dose is administered at a different time in one day. Alternatively, a 250 mg dose administered twice daily can be administered 125 mg BID (i.e., two 125 mg doses administered at different times in one day).

In some embodiments, Compound 2, or a pharmaceutically acceptable composition thereof, is administered three times a day. In some embodiments, Compound 2, or a pharmaceutically acceptable composition thereof, is administered “TID”, or three equivalent doses administered at three different times in one day. In some embodiments, Compound 2, or a pharmaceutically acceptable composition thereof, is administered in three different doses, wherein at least one of the administered doses differs from another administered dose. In some embodiments, Compound 2, or a pharmaceutically acceptable composition thereof, is administered four times a day. In some embodiments, Compound 2, or a pharmaceutically acceptable composition thereof, is administered “QID”, or four equivalent doses administered at four different times in one day. In some embodiments, Compound 2, or a pharmaceutically acceptable composition thereof, is administered in four different doses, wherein at least one of the administered doses differs from another administered dose.

In some embodiments, a unit dose of Compound 2 is administered once a day (QD). In some embodiments, a unit dose of Compound 2 is administered twice a day. In some embodiments, a unit dose of Compound 2 is administered BID.

In some embodiments, a pharmaceutically acceptable composition comprises a therapeutically effective amount of Compound 2, wherein the therapeutically effective amount is a total daily dose selected from about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, about 1000 mg, about 1005 mg, about 1010 mg, about 1015 mg, about 1020 mg, about 1025 mg, about 1030 mg, about 1035 mg, about 1040 mg, about 1045 mg, about 1050 mg, about 1055 mg, about 1060 mg, about 1065 mg, about 1070 mg, about 1075 mg, about 1080 mg, about 1085 mg, about 1090 mg, about 1095 mg, about 1100 mg, about 1105 mg, about 1110 mg, about 1115 mg, about 1120 mg, about 1125 mg, about 1130 mg, about 1135 mg, about 1140 mg, about 1145 mg, about 1150 mg, about 1155 mg, about 1160 mg, about 1165 mg, about 1170 mg, about 1175 mg, about 1180 mg, about 1185 mg, about 1190 mg, about 1195 mg, about 1200 mg, about 1205 mg, about 1210 mg, about 1215 mg, about 1220 mg, about 1225 mg, about 1230 mg, about 1235 mg, about 1240 mg, about 1245 mg, about 1250 mg, about 1255 mg, about 1260 mg, about 1265 mg, about 1270 mg, about 1275 mg, about 1280 mg, about 1285 mg, about 1290 mg, about 1295 mg, about 1300 mg, about 1305 mg, about 1310 mg, about 1315 mg, about 1320 mg, about 1325 mg, about 1330 mg, about 1335 mg, about 1340 mg, about 1345 mg, about 1350 mg, about 1355 mg, about 1360 mg, about 1365 mg, about 1370 mg, about 1375 mg, about 1380 mg, about 1385 mg, about 1390 mg, about 1395 mg, about 1400 mg, about 1405 mg, about 1410 mg, about 1415 mg, about 1420 mg, about 1425 mg, about 1430 mg, about 1435 mg, about 1440 mg, about 1445 mg, about 1450 mg, about 1455 mg, about 1460 mg, about 1465 mg, about 1470 mg, about 1475 mg, about 1480 mg, about 1485 mg, about 1490 mg, about 1495 mg, about 1500 mg, about 1505 mg, about 1510 mg, about 1515 mg, about 1520 mg, about 1525 mg, about 1530 mg, about 1535 mg, about 1540 mg, about 1545 mg, about 1550 mg, about 1555 mg, about 1560 mg, about 1565 mg, about 1570 mg, about 1575 mg, about 1580 mg, about 1585 mg, about 1590 mg, about 1595 mg, about 1600 mg, about 1605 mg, about 1610 mg, about 1615 mg, about 1620 mg, about 1625 mg, about 1630 mg, about 1635 mg, about 1640 mg, about 1645 mg, about 1650 mg, about 1655 mg, about 1660 mg, about 1665 mg, about 1670 mg, about 1675 mg, about 1680 mg, about 1685 mg, about 1690 mg, about 1695 mg, about 1700 mg, about 1705 mg, about 1710 mg, about 1715 mg, about 1720 mg, about 1725 mg, about 1730 mg, about 1735 mg, about 1740 mg, about 1745 mg, about 1750 mg, about 1755 mg, about 1760 mg, about 1765 mg, about 1770 mg, about 1775 mg, about 1780 mg, about 1785 mg, about 1790 mg, about 1795 mg, about 1800 mg, about 1805 mg, about 1810 mg, about 1815 mg, about 1820 mg, about 1825 mg, about 1830 mg, about 1835 mg, about 1840 mg, about 1845 mg, about 1850 mg, about 1855 mg, about 1860 mg, about 1865 mg, about 1870 mg, about 1875 mg, about 1880 mg, about 1885 mg, about 1890 mg, about 1895 mg, about 1900 mg, about 1905 mg, about 1910 mg, about 1915 mg, about 1920 mg, about 1925 mg, about 1930 mg, about 1935 mg, about 1940 mg, about 1945 mg, about 1950 mg, about 1955 mg, about 1960 mg, about 1965 mg, about 1970 mg, about 1975 mg, about 1980 mg, about 1985 mg, about 1990 mg, about 1995 mg, about 2000 mg, about 2005 mg, about 2010, about 2015 mg, about 2020 mg, about 2025 mg, about 2030 mg, about 2035, about 2040 mg, about 2045 mg, about 2050 mg, about 2055 mg, about 2060 mg, about 2065 mg, about 2070 mg, about 2075 mg, about 2080 mg, about 2085 mg, about 2090 mg, about 2095 mg, about 2100 mg, about 2105 mg, about 2110 mg, about 2115 mg, about 2120 mg, about 2125 mg, about 2130 mg, about 2135 mg, about 2140 mg, about 2145 mg, about 2150 mg, about 2155 mg, about 2160 mg, about 2165 mg, about 2170 mg, about 2175 mg, about 2180 mg, about 2185 mg, about 2190 mg, about 2195 mg, about 2200 mg, about 2205 mg, about 2210 mg, about 2215 mg, about 2220 mg, about 2225 mg, about 2230 mg, about 2235 mg, about 2240 mg, about 2245 mg, about 2250 mg, about 2255 mg, about 2260 mg, about 2265 mg, about 2270 mg, about 2275 mg, about 2280 mg, about 2285 mg, about 2290 mg, about 2295 mg, about 2300 mg, about 2305 mg, about 2310 mg, about 2315 mg, about 2320 mg, about 2325 mg, about 2330 mg, about 2335 mg, about 2340 mg, about 2345 mg, about 2350 mg, about 2355 mg, about 2360 mg, about 2365 mg, about 2370 mg, about 2375 mg, about 2380 mg, about 2385 mg, about 2390 mg, about 2395 mg, about 2400 mg, about 2405 mg, about 2410 mg, about 2415 mg, about 2420 mg, about 2425 mg, about 2430 mg, about 2435 mg, about 2440 mg, about 2445 mg, about 2450 mg, about 2455 mg, about 2460 mg, about 2465 mg, about 2470 mg, about 2475 mg, about 2480 mg, about 2485 mg, about 2490 mg, about 2495 mg, about 2500 mg, about 2505 mg, about 2510 mg, about 2515 mg, about 2520 mg, about 2525 mg, about 2530 mg, about 2535 mg, about 2540 mg, about 2545 mg, about 2550 mg, about 2555 mg, about 2560 mg, about 2565 mg, about 2570 mg, about 2575 mg, about 2580 mg, about 2585 mg, about 2590 mg, about 2595 mg, about 2600 mg, about 2605 mg, about 2610 mg, about 2615 mg, about 2620 mg, about 2625 mg, about 2630 mg, about 2635 mg, about 2640 mg, about 2645 mg, about 2650 mg, about 2655 mg, about 2660 mg, about 2665 mg, about 2670 mg, about 2675 mg, about 2680 mg, about 2685 mg, about 2690 mg, about 2695 mg, about 2700 mg, about 2705 mg, about 2710 mg, about 2715 mg, about 2720 mg, about 2725 mg, about 2730 mg, about 2735 mg, about 2740 mg, about 2745 mg, about 2750 mg, about 2755 mg, about 2760 mg, about 2765 mg, about 2770 mg, about 2775 mg, about 2780 mg, about 2785 mg, about 2790 mg, about 2795 mg, about 2800 mg, about 2805 mg, about 2810 mg, about 2815 mg, about 2820 mg, about 2825 mg, about 2830 mg, about 2835 mg, about 2840 mg, about 2845 mg, about 2850 mg, about 2855 mg, about 2860 mg, about 2865 mg, about 2870 mg, about 2875 mg, about 2880 mg, about 2885 mg, about 2890 mg, about 2895 mg, about 2900 mg, about 2905 mg, about 2910 mg, about 2915 mg, about 2920 mg, about 2925 mg, about 2930 mg, about 2935 mg, about 2940 mg, about 2945 mg, about 2950 mg, about 2955 mg, about 2960 mg, about 2965 mg, about 2970 mg, about 2975 mg, about 2980 mg, about 2985 mg, about 2990 mg, about 2995 mg, about 3000 mg, about 3005 mg, about 3010 mg, about 3015 mg, about 3020 mg, about 3025 mg, about 3030 mg, about 3035 mg, about 3040 mg, about 3045 mg, about 3050 mg, about 3055 mg, about 3060 mg, about 3065 mg, about 3070 mg, about 3075 mg, about 3080 mg, about 3085 mg, about 3090 mg, about 3095 mg, about 3100 mg, about 3105 mg, about 3110 mg, about 3115 mg, about 3120 mg, about 3125 mg, about 3130 mg, about 3135 mg, about 3140 mg, about 3145 mg, about 3150 mg, about 3155 mg, about 3160 mg, about 3165 mg, about 3170 mg, about 3175 mg, about 3180 mg, about 3185 mg, about 3190 mg, about 3195 mg, about 3200 mg, about 3205 mg, about 3210 mg, about 3215 mg, about 3220 mg, about 3225 mg, about 3230 mg, about 3235 mg, about 3240 mg, about 3245 mg, about 3250 mg, about 3255 mg, about 3260 mg, about 3265 mg, about 3270 mg, about 3275 mg, about 3280 mg, about 3285 mg, about 3290 mg, about 3295 mg, about 3300 mg, about 3305 mg, about 3310 mg, about 3315 mg, about 3320 mg, about 3325 mg, about 3330 mg, about 3335 mg, about 3340 mg, about 3345 mg, about 3350 mg, about 3355 mg, about 3360 mg, about 3365 mg, about 3370 mg, about 3375 mg, about 3380 mg, about 3385 mg, about 3390 mg, about 3395 mg, about 3400 mg, about 3405 mg, about 3410 mg, about 3415 mg, about 3420 mg, about 3425 mg, about 3430 mg, about 3435 mg, about 3440 mg, about 3445 mg, about 3450 mg, about 3455 mg, about 3460 mg, about 3465 mg, about 3470 mg, about 3475 mg, about 3480 mg, about 3485 mg, about 3490 mg, about 3495 mg, about 3500 mg, about 3505 mg, about 3510 mg, about 3515 mg, about 3520 mg, about 3525 mg, about 3530 mg, about 3535 mg, about 3540 mg, about 3545 mg, about 3550 mg, about 3555 mg, about 3560 mg, about 3565 mg, about 3570 mg, about 3575 mg, about 3580 mg, about 3585 mg, about 3590 mg, about 3595 mg, about 3600 mg, about 3605 mg, about 3610 mg, about 3615 mg, about 3620 mg, about 3625 mg, about 3630 mg, about 3635 mg, about 3640 mg, about 3645 mg, about 3650 mg, about 3655 mg, about 3660 mg, about 3665 mg, about 3670 mg, about 3675 mg, about 3680 mg, about 3685 mg, about 3690 mg, about 3695 mg, about 3700 mg, about 3705 mg, about 3710 mg, about 3715 mg, about 3720 mg, about 3725 mg, about 3730 mg, about 3735 mg, about 3740 mg, about 3745 mg, about 3750 mg, about 3755 mg, about 3760 mg, about 3765 mg, about 3770 mg, about 3775 mg, about 3780 mg, about 3785 mg, about 3790 mg, about 3795 mg, about 3800 mg, about 3805 mg, about 3810 mg, about 3815 mg, about 3820 mg, about 3825 mg, about 3830 mg, about 3835 mg, about 3840 mg, about 3845 mg, about 3850 mg, about 3855 mg, about 3860 mg, about 3865 mg, about 3870 mg, about 3875 mg, about 3880 mg, about 3885 mg, about 3890 mg, about 3895 mg, about 3900 mg, about 3905 mg, about 3910 mg, about 3915 mg, about 3920 mg, about 3925 mg, about 3930 mg, about 3935 mg, about 3940 mg, about 3945 mg, about 3950 mg, about 3955 mg, about 3960 mg, about 3965 mg, about 3970 mg, about 3975 mg, about 3980 mg, about 3985 mg, about 3990 mg, about 3995 mg or about 4000 mg.

In some embodiments, Compound 2 is administered BID, wherein the BID dose is selected from 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1700 mg, 1750 mg, 1800 mg, 1850 mg, 1900 mg, 1950 mg or 2000 mg. In some embodiments, Compound 2 is administered 300 mg BID. In some embodiments, Compound 2 is administered 500 mg BID. In some embodiments, Compound 2 is administered 625 mg BID. In some embodiments, Compound 2 is administered 700 mg BID. In some embodiments, Compound 2 is administered 750 mg BID. In some embodiments, Compound 2 is administered 1000 mg BID.

In some embodiments, the predominant dose limiting toxicity of adminstration of Compound 2 is hyperglycemia. Generally, plasma glucose levels normaize if Compound 2 is temporarily interrupted, or if hypoglycemia medication is co-administered. Any hypoglycemic medication is expected to be acceptable and include agents such as insulin, metformin, glipizide, and the like. Alternatively, in some embodiments, a dose reduction can also address any hyperglycemia. Common side effects of other EGFR inhibitors, notably rash and diarrhea are not typically observed with administration of Compound 2.

According to one embodiment, the invention relates to a method of inhibiting protein kinase activity in a biological sample comprising the step of contacting said biological sample with Compound 2 or a composition comprising said compound.

According to another embodiment, the invention relates to a method of inhibiting at least one mutant of EGFR (e.g., a deletion mutation, an activating mutation, a resistant mutations, or combination thereof) activity in a biological sample comprising the step of contacting said biological sample with Compound 2, or a composition comprising the compound. In certain embodiments, the invention relates to a method of irreversibly inhibiting at least one mutant of EGFR (e.g., a deletion mutation, an activating mutation, a resistant mutation, or combination thereof) activity in a biological sample comprising the step of contacting the biological sample with Compound 2, or a composition comprising the compound.

In certain embodiments, Compound 2 selectively inhibits in a biological sample (a) at least one activating mutation, (b) T790M, and (c) is sparing as to WT. In some embodiments, an at least one activating mutation is a deletion mutation. In some embodiments, an at least one activating mutation is a point mutation. In some embodiments, an activating mutation is delE746-A750. In some embodiments, an activating mutation is L858R. In some embodiments, an activating mutation is G719S.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of at least one mutant of EGFR (e.g., a deletion mutation, an activating mutation, a resistant mutation, or combination thereof) activity in a biological sample is useful for a variety of purposes that are known to one of skill in the art. Examples of such purposes include, but are not limited to, blood transfusion, organ transplantation, biological specimen storage, and biological assays.

Another embodiment of the present invention relates to a method of inhibiting at least one mutant of EGFR (e.g., a deletion mutation, an activating mutation, a resistant mutation, or combination thereof) activity in a patient comprising the step of administering to the patient Compound 2 or a composition comprising the compound. In certain embodiments, the present invention provides a method for inhibiting (a) at least one activating mutation, and (b) T790M in a patient, and (c) is sparing as to WT, wherein the method comprises administering to the patient Compound 2 or composition thereof. In some embodiments, an at least one activating mutation is a deletion mutation. In some embodiments, an at least one activating mutation is a point mutation. In some embodiments, the present invention provides a method for inhibiting at least one mutant of EGFR in a patient, wherein an activating mutation is delE746-A750. In some embodiments, the present invention provides a method for inhibiting at least one mutant of EGFR in a patient, wherein an activating mutation is L858R. In some embodiments, the present invention provides a method for inhibiting at least one mutant of EGFR in a patient, wherein an activating mutation is G719S.

According to another embodiment, the invention relates to a method of inhibiting at least one mutant of EGFR (e.g., a deletion mutation, an activating mutation, a resistant mutation, or combination thereof) activity in a patient comprising the step of administering to the patient Compound 2 or a composition comprising the compound. According to certain embodiments, the invention relates to a method of irreversibly inhibiting at least one mutant of EGFR activity (e.g., a deletion mutation, an activating mutation, a resistant mutation, or combination thereof) in a patient comprising the step of administering to said patient Compound 2 or a composition comprising the compound. In certain embodiments, the present invention provides a method for irreversibly inhibiting (a) at least one activating mutation, and (b) T790M in a patient, and (c) is sparing as to WT, wherein said method comprises administering to the patient Compound 2 or composition thereof. In some embodiments, an irreversibly inhibited at least one activating mutation is a deletion mutation. In some embodiments, an irreversibly inhibited at least one activating mutation is a point mutation. In some embodiments, the present invention provides a method for irreversibly inhibiting at least one mutant of EGFR in a patient, wherein an activating mutation is delE746-A750. In some embodiments, the present invention provides a method for irreversibly inhibiting at least one mutant of EGFR in a patient, wherein an activating mutation is L858R. In some embodiments, the present invention provides a method for irreversibly inhibiting at least one mutant of EGFR in a patient, wherein an activating mutation is G719S.

In other embodiments, the present invention provides a method for treating a disorder mediated by one or more of at least one mutant of EGFR (e.g., a deletion mutation, an activating mutation, a resistant mutation, or combination thereof) in a patient in need thereof, comprising the step of administering to said patient Compound 2 or pharmaceutically acceptable composition thereof. Such disorders are described in detail herein.

Depending upon the particular condition, or disease, to be treated, additional therapeutic agents, which are normally administered to treat that condition, may also be present in the compositions of this invention or as part of a treatment regimen including Compound 2. As used herein, additional therapeutic agents that are normally administered to treat a particular disease, or condition, are known as “appropriate for the disease or condition being treated.”

For example, Compound 2 or a pharmaceutically acceptable composition thereof is administered in combination with chemotherapeutic agents to treat proliferative diseases and cancer. Examples of known chemotherapeutic agents include, but are not limited to, Adriamycin, dexamethasone, vincristine, cyclophosphamide, fluorouracil, topotecan, taxol, interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluorouracil, campthothecin, cisplatin, metronidazole, and Gleevec™, among others. In other embodiments, Compound 2 is administered in combination with a biologic agent, such as Avastin or VECTIBIX.

In certain embodiments, Compound 2 or a pharmaceutically acceptable composition thereof is administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG Live, bevacuzimab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, erlotinib, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

Other examples of agents the inhibitors of this invention may also be combined with include, without limitation: treatments for Alzheimer's Disease such as donepezil hydrochloride (Aricept®) and rivastigmine (Exelon®); treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), glatiramer acetate (Copaxone), and mitoxantrone; treatments for asthma such as albuterol and montelukast (Singulair®); agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

In certain embodiments, Compound 2 or a pharmaceutically acceptable composition thereof is administered in combination with a monoclonal antibody or an siRNA therapeutic.

The additional agents may be administered separately from a Compound 2-containing composition, as part of a multiple dosage regimen. Alternatively, those agents may be part of a single dosage form, mixed together with Compound 2 in a single composition. If administered as part of a multiple dosage regime, the two active agents may be submitted simultaneously, sequentially or within a period of time from one another (e.g., one hour, two hours, six hours, twelve hours, one day, one week, two weeks, one month).

As used herein, the terms “combination,” “combined,” and related terms refer to the simultaneous or sequential administration of therapeutic agents in accordance with this invention. For example, Compound 2 may be administered with another therapeutic agent simultaneously or sequentially in separate unit dosage forms or together in a single unit dosage form. Accordingly, the present invention provides a single unit dosage form comprising Compound 2, an additional therapeutic agent, and a pharmaceutically acceptable carrier, adjuvant, or vehicle.

The amount of Compound 2 and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of Compound 2 can be administered.

In those compositions that include an additional therapeutic agent, that additional therapeutic agent and Compound 2 may act synergistically. Therefore, the amount of additional therapeutic agent in such compositions may be less than that required in a monotherapy utilizing only that therapeutic agent. In such compositions, a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compound 2 or pharmaceutical compositions thereof may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with Compound 2 are another embodiment of the present invention.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### Exemplification

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Preparation of Compound 1

The synthesis of Compound 1 is described in detail at Example 3 of the '061 application.

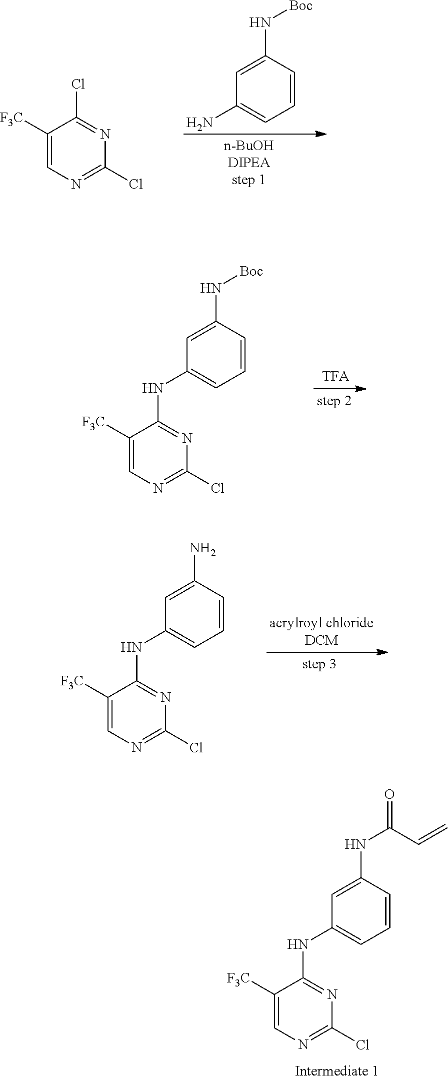 [SMILES: FC(F)(F)c1cnc(Cl)nc1Cl ; CC(C)(C)OC(=O)Nc1cccc(N)c1 ; CC(C)(C)OC(=O)Nc1cccc(Nc2nc(Cl)ncc2C(F)(F)F)c1 ; Nc1cccc(Nc2nc(Cl)ncc2C(F)(F)F)c1 ; C=CC(=O)Nc1cccc(Nc2nc(Cl)ncc2C(F)(F)F)c1]

### Step 1:

In a 25 mL 3-neck round-bottom flask previously equipped with a magnetic stirrer, Thermo pocket and CaCl₂ guard tube, N-Boc-1,3-diaminobenzene (0.96 g) and n-butanol (9.00 mL) were charged. The reaction mixture was cooled to 0° C. 2,4-Dichloro-5-trifluoromethylpyrimidine (1.0 g) was added dropwise to the above reaction mixture at 0° C. Diisopropylethylamine (DIPEA) (0.96 mL) was dropwise added to the above reaction mixture at 0° C. and the reaction mixture was stirred for 1 hr at 0° C. to 5° C. Finally, the reaction mixture was allowed to warm to room temperature. The reaction mixture was stirred for another 4 hrs at room temperature. Completion of reaction was monitored by TLC using hexane: ethyl acetate (7:3). The solid precipitated out was filtered off and washed with 1-butanol (2 mL). The solid was dried under reduced pressure at 40° C. for 1 hr. ¹H-NMR (DMSO-d6, 400 MHz) δ 1.48 (S, 9H), 7.02 (m, 1H), 7.26 (m, 2H), 7.58 (S, 1H), 8.57 (S, 1H), 9.48 (S, 1H), 9.55 (S, 1H).

### Step 2:

To the above crude (3.1 g) in dichloromethane (DCM) (25 mL) was added trifluoroacetic acid (TFA) (12.4 mL) slowly at 0° C. The reaction mixture was allowed to warm to room temperature. The reaction mixture was stirred for another 10 min at room temperature. The crude was concentrated under reduced pressure.

### Step 3:

The concentrated crude was dissolved in DIPEA (2.0 mL) and dichloromethane (25 mL), and then cooled to −30° C. To the reaction mixture was slowly added acryloyl chloride (0.76 g) at −30° C. The reaction mass was warmed to room temperature stirred at room temperature for 1.0 hr. The reaction was monitored on TLC using hexane: ethyl acetate (7:3) as mobile phase. The reaction was completed after 1 hr. Step 3 yielded intermediate 1.

### Step 4:

To obtain a salt of compound 1, a mixture of intermediate 1 (16 mg) and 2-methoxy-4-(4-acetylpiperazinyl)aniline in dioxane (1.0 mL) with catalytic trifluoroacetic acid was stirred overnight at 50° C. The crude was concentrated under reduced pressure and purified using HPLC (TFA modifier) to give compound 1 as a TFA salt. ¹H-NMR (DMSO-d6, 400 MHz) δ 10.2 (S, 1H), 8.2 (br, 1H), 8.30 (S, 1H), 7.73 (br, 1H), 7.52 (d, J=7.8 Hz, 1H), 7.45 (d, J=7.8 Hz, 1H), 7.26 (J=8.2 Hz, 1H), 7.14 (be, 1H), 6.60 (S, 1H), 6.42 (dd, J=11.4, 16.9 Hz, 1H), 6.24 (d, J=16.9 Hz, 1H), 5.75 (d, J=11.4 Hz, 1H), 3.76 (S, 3H), 3.04 (br, 4H), 2.04 (S, 3H); calculated mass for C₂₇H₂₈F₃N₇O₃: 555.2. found: 556.2 (M+H⁺).

### Step 5:

To obtain the free base form of Compound 1 from the TFA salt, the salt was added to DCM and cooled to 0° C. Na₂CO₃ solution (9.6% w/w) was added at 0° C. The mixture was warmed to 20° C. and stirred for 35 min. The pH of the aqueous layer was >8. The layers were separated. Extraction of the aqueous layer was performed using DCM. The organic layers were combined and washed with brine. The organic layer was collected and evaporated to yield a solid of Compound 1.

### General Preparation of Compound 2

For each counterion and solvent system, ca. 25 or 50 mg of the free base of Compound 1 was slurried in 200-300 μl of the allocated solvent. The solvents included acetone, dichloromethane, cyclohexane, ethyl acetate, methanol (methyl ethyl ketone for sulfonic acid-containing counterions), methyl isobutyl ketone, 2-propanol (isopropyl acetate for sulfonic acid-containing counterions), tetrahydrofuran and acetonitrile:water (90:10). The respective counterion was also dissolved/slurried in 200-300 μl of the allocated solvent. The counterions included benzenesulfonic acid, camphor sulfonic acid, 1,2-ethane disulfonic acid, hydrobromic acid, hydrochloric acid, maleic acid, methanesulfonic acid, naphthalene-2-sulfonic acid, 1,5-naphthalene disulfonic acid, oxalic acid, 4-toluenesulfonic acid and 2,4,6-trihydroxybenzoic acid. One equivalent of each counterion was used and additional experiments with two equivalents of benzenesulfonic acid, hydrochloric acid, sulphuric acid and p-toluenesulfonic acid were performed. The acid solution/slurry was then added to the slurry of Compound 1 in small aliquots in order to minimize the risk of degradation. The pH of the reaction was then checked using universal indicator paper.

The mixtures of Compound 1/counterion/solvent created using the procedure above were temperature cycled between ca. 0° C. and ambient (ca. 22° C.) whilst stirring in 1 hour cycles for a period of 1-2 days. Overnight, samples were kept at ca. 2-5° C. The mixtures were visually checked for any obvious signs of degradation (i.e. color changes) and then, if not visually degraded, any solids present were isolated and allowed to dry at ambient conditions prior to analysis. The solids represent isolated Compound 2.

### General Procedures

The solubility of the potential salts was tested using a shake flask method whereby a slurry of each salt was prepared in deionized water and the pH of the reaction was reduced to below pH 2 by adding a small amount of the counterion used for salt formation. The pH was tested using universal indicator paper. After ca. 24 hours of shaking, the slurries were filtered for the solubility determination using HPLC analysis.

X-Ray Powder Diffraction.

X-ray powder diffraction (XRPD) analysis was carried out on a Siemens D5000, scanning the samples between 3 and 30, 35 or 50° 2θ. For samples <100 mg, ca. 5-10 mg of sample was gently compressed onto a glass slide which fitted into the sample holder. For samples >100 mg, ca. 100 mg of sample was gently compressed into a plastic sample holder, so that the sample surface was smooth and just above the level of the sample holder. Measurements were made using the following experimental conditions:

| start position | 3.00 °2Θ | |
|---|---|---|
| end position | 30, 35 or 50 °2Θ | |
| step size | 0.02 °2Θ | |
| scan step time | 1 | s |
| scan type | continuous | |
| offset | 0 °2Θ | |
| divergence slit type | fixed | |
| divergence slit size | 2.0000° | |
| receiving slit size | 0.2 | mm |
| temperature | 20° | C. |
| anode material | copper (Cu) | |
| K-Alpha1 | 1.54060 | Angstroms |
| K-Alpha2 | 1.54443 | Angstroms |
| K-Beta | 1.39225 | Angstroms |

| | |
|---|---|
| K-A2/K-A1 Ratio | 0.50000 |
| Generator settings | 40 mA, 40 kV |
| goniometer radius | 217.50 |

Polarized Light Microscopy.

In polarized light microscopy (PLM), the presence of crystallinity (birefringence) was determined using an Olympus BX50 polarising microscope, equipped with a Motic camera and image capture software (Motic Images Plus 2.0). All images were recorded using the 20× objective, unless otherwise stated.

Thermogravimetric Analysis.

For thermogravimetric analysis (TGA), approximately 5-10 mg of material was accurately weighed into an open aluminium pan and loaded into a simultaneous thermogravimetric/differential thermal analyser (TG/DTA) and held at room temperature. The sample was then heated at a rate of 10° C./min from 25° C. to 300° C. during which time the change in sample weight was recorded along with any differential thermal events (DTA). Nitrogen was used as the purge gas, at a flow rate of 100 cm³/min.

Differential Scanning calorimetry.

For differential scanning calorimetry (DSC), approximately 5-10 mg of material was weighed into an aluminium DSC pan and sealed non-hermetically with a pierced aluminium lid. The sample pan was then loaded into a Seiko DSC6200 (equipped with a cooler) cooled and held at 25° C. Once a stable heat-flow response was obtained, the sample and reference were heated to ca. 260° C.-280° C. at scan rate of 10° C./min and the resulting heat flow response monitored.

Nuclear Magnetic Resonance Spectroscopy.

¹H-NMR experiments were performed on a Bruker AV400 (¹H frequency: 400 MHz). ¹H experiments of each sample were performed in deuterated DMSO and each sample was prepared to ca. 1 mg/mL concentration.

Dynamic Vapour Sorption.

For dynamic vapour sorption (DVS), approximately 10-20 mg of sample was placed into a wire mesh vapour sorption balance pan and loaded into a DVS-1 dynamic vapour sorption balance by Surface Measurement Systems. The sample was subjected to a ramping profile from 20-90% relative humidity (RH) at 10% increments, maintaining the sample at each step until a stable weight had been achieved (99.5% step completion). After completion of the sorption cycle, the sample was dried using the same procedure, but all the way down to 0% RH and finally taken back to the starting point of 20% RH. The weight change during the sorption/desorption cycles were plotted, allowing for the hygroscopic nature of the sample to be determined.

Infrared Spectroscopy.

Infrared spectroscopy (IR) was carried out on a Bruker ALPHA P spectrometer. Sufficient material was placed onto the centre of the plate of the spectrometer and the spectra were obtained using the following parameters:

| | | |
|---|---|---|
| resolution | 4 | cm⁻¹ |
| background scan time | 16 | scans |
| sample scan time | 16 | scans |

| | |
|---|---|
| data collection | 4000 to 400 cm⁻¹ |
| result spectrum | transmittance |
| software | OPUS version 6 |

For Karl Fischer (KF) Coulometric titration, 10-15 mg of solid material was accurately weighed into a vial. The solid was then manually introduced into the titration cell of a Mettler Toledo C30 Compact Titrator. The vial was back-weighed after the addition of the solid and the weight of the added solid entered on the instrument. Titration was initiated once the sample had fully dissolved in the cell. The water content was calculated automatically by the instrument as a percentage and the data printed.

Reverse-phase gradient high performance liquid chromatography (HPLC) was performed on an Agilent 1100 instrument fitted with a C18, 3.0×100 mm×3.5 μm column. The detection wavelength was 240 nm.

A Sotax AT7 dissolution bath (USP 2, EP 2 apparatus) was used for the dissolution study in which paddles were used to stir the media. All tests were carried out at 37° C. and a paddle speed of 100 rpm.

### Example 1

### Primary Salt Screen

The results of the primary salt screen, based on the general preparation of Compound 2, are shown in Table 1. Table 1 indicates the counterion, the solvent and the solid form(s) obtained.

TABLE 1
Results of Primary Salt Screen

| | | | | | | | | 2- | | |
|---|---|---|---|---|---|---|---|---|---|---|
| | | | | | | | | Propanol | | Aceto- |
| | Equiv- | | | | | | Methyl- | (IPA) or | | nitrile: |
| | alents | | Dichloro- | Cyclo- | Ethyl | Methanol | isobutyl | Isopropyl | Tetra- | Water |
| Counterion | of Acid | Acetone | methane | hexane | Acetate | or MEK | Ketone | acetate | hydrofuran | (10%) |
| 2,4,6-Trihydroxybenzoic | 1 | FC | FC FA | FC FA | AM | S1 | FC | FC | S2 | S2 |
| Benzenesulfonic acid | 1 | S1 | XR | FC | FC | S1 S2 | S2 | S2 | S1 FC | XR |
| Benzenesulfonic acid | 2 | S1 | AM | FC | S1 | S1 | FC | FC | S1 | S2 |
| Hydrochloric acid | 1 | AM | FC | FC | FC | FC | AM | FC | FC | S1 |
| Hydrochloric acid | 2 | AM | S1 | FC | XR | S1 XR | S1 S2 | S XR | AM | S1 |
| Maleic acid | 1 | XR | S1 | FC FA | XR XR | S2 | FC | FC | S3 | S2 XR |
| Methanesulfonic acid | 2 | S1 | AM | FC | AM | S1 | AM | AM | S1 | AM |
| Oxalic acid | 1 | S1 | AM | FC FA | S1 | S1 | AM | AM | AM | S2 |
| Sulfuric acid | 1 | GM | FC | FC | FC | AM | FC | AM | FC SP | AM |
| Sulfuric acid | 2 | FC | FC | FC | FC | FC | FC | FC | FC | FC |
| p-Toluenesulfonic acid | 1 | S1 | S1 | FC | S1 | S1 | S1 | FC | S2 | AM |
| monohydrate | | | | | | | | | | |
| p-Toluenesulfonic acid | 2 | S1 | S1 | FC | S1 | S2 | AM | AM | S2 XR | AM |
| monohydrate | | | | | | | | | | |
| 1,2-Ethane disulfonic acid | 1 | AM | AM | FC | FC | S1 | FC | FC | AM | S2 |
| dehydrate | | | | | | | | | | |
| 1,2-Ethane disulfonic acid | 2 | AM | AM | FC | AM | S1 | FC | FC | AM | S2 |
| dehydrate | | | | | | | | | | |
| Hydrobromic acid | 1 | AM | S1 | FC/S1 | S1 | S1(?) | S1 | S1 | S1 | S1 |
| Hydrobromic acid | 2 | S1/XR | S1 | S1 | S1 | S1 | S1 | S1 | S2 | S1 |
| Naphthalene-2-sulfonic acid | 1 | S1 | S1 | FC | S1 | S1 | S1 | S1 | AM | S1 |
| Naphthalene-2-sulfonic acid | 2 | S1 | S2 | FC | AM | S2 | XR | AM | S3 | S4 |
| 1,5-Naphthalene disulfonic | 1 | AM | FC | FC | FC | XR | FC | FC | FC | S1 |
| acid | | | | | | | | | | |
| 1,5-Naphthalene disulfonic | 2 | XR | AM | FA | FA/FC | FA/FC | XR | FA | AM | S1 |
| acid | | | | | | | | | | |
| Camphor-10-sulfonic acid | 1 | S1 | S1 | FC | S1 | S1 | S1 | S1 | S1 | S2 |
| Camphor-10-sulfonic acid | 2 | AM | AM | FA/FC | XR/S1 | S1/XR | AM | FC | AM | AM |

S1 = salt, polymorphic form 1
S2 = salt, polymorphic form 2
S3 = salt, polymorphic form 3
S4 = salt, polymorphic form 4
SP = salt, partially crystalline
FA = free acid
FC = free Compound 1
XR = different XRPD pattern, but only a few peaks in the diffractogram (possibly indicating degradation)
AM = amorphous
GM = solid that rapidly converts to gum upon isolation

### Example 2

### Primary Salt Screen

For the potential salts obtained during the primary salt screen in Example 1, the samples were set-up for 1 week stability studies at 40° C./75% RH (open vials) and 80° C. (open vials). TGA was carried out after the stability studies for samples containing sufficient material. The solubility of the samples was also tested in an aqueous medium (pH<2). The results for the stability and solubility studies are indicated in Table 2.

TABLE 2
Stability and solubility results from potential salts obtained in the primary salt screen

| | | Approximate | | |
|---|---|---|---|---|
| | | Solubility | 40° C./75% RH | 80° C. (open |
| Potential Salt | Form | (mg/ml) | (open conditions) | conditions) |
| 2,4,6- | Form I | Below LOQ | No form change, | No form change, |
| Trihydroxybenzoate | | | remains predominantly | remains predominantly |
| | | | crystalline | crystalline |
| 2,4,6- | Form II | Below LOQ | No form change, but | No form change, but |
| Trihydroxybenzoate | | | poor crystallinity | poor crystallinity |
| Besylate (1 equiv.) | Form I | 0.047 | No form change, | No form change, |
| | | | remains predominantly | remains predominantly |
| | | | crystalline | crystalline |
| Besylate (1 equiv.) | Form II | 0.055 | No form change, some | No form change, some |
| | | | decline in crystallinity. | decline in crystallinity. |
| Besylate (2 equiv.) | Form I | 4.264 | Solid/gum present. | No form change, |
| | | | Change in polymorphic | remains predominantly |
| | | | form (likely hydrated), | crystalline. TGA shows |
| | | | but poor crystallinity. | initial 1.95% weight |
| | | | | loss likely due to |
| | | | | unbound volatiles. No |
| | | | | further weight losses |
| | | | | present prior to likely |
| | | | | degradation. |
| Besylate (2 equiv.) | Form II | 0.044 | No form change, | No form change, |
| | | | remains predominantly | remains predominantly |
| | | | crystalline | crystalline |
| Hydrochloride | Form I | 0.400 | No form change, | No form change, |
| (1 equiv.) | | | partially crystalline. | poorly crystalline |
| Hydrochloride | Form I | 0.196 | No form change, | No form change, |
| (2 equiv.) | | | remains predominantly | remains predominantly |
| | | | crystalline. TGA shows | crystalline |
| | | | initial weight loss of ca. | |
| | | | 2% likely due to | |
| | | | unbound volatiles. A | |
| | | | further ca. 8% weight | |
| | | | loss from ca. 150° C., | |
| | | | directly followed by | |
| | | | further weight loss likely | |
| | | | due to degradation. | |
| | | | Likely hydrated or | |
| | | | solvated. | |
| Maleate | Form I | 0.168 | No form change, | No form change, |
| | | | remains predominantly | remains predominantly |
| | | | crystalline. TGA shows | crystalline. |
| | | | a weight loss of ca. 11% | |
| | | | from ca. 160° C. The | |
| | | | required DCM content | |
| | | | for a mono DCM solvate | |
| | | | is 11.23%. | |
| Maleate | Form II | 0.271 | No form change, | No form change, |
| | | | remains predominantly | remains predominantly |
| | | | crystalline. TGA shows | crystalline |
| | | | an initial 1.76% weight | |
| | | | loss likely due to | |
| | | | unbound volatiles. A | |
| | | | further ca. 6.3% weight | |
| | | | loss is seen from ca. | |
| | | | 130° C., likely due to | |
| | | | bound solvent/water. | |
| Maleate | Form III | 0.251 | No clear form change | No clear form change |
| | | | but poorly crystalline | but poorly crystalline |
| | | | (brown) | (brown) |
| Mesylate | Form I | 7.286 | Gum formed | Amorphous solid |
| Oxalate | Form I | 0.158 | No form change, | No form change, |
| | | | remains predominantly | remains predominantly |
| | | | crystalline. TGA shows | crystalline. |
| | | | a weight loss of ca. | |
| | | | 14.4% associated with | |
| | | | an endotherm at ca. | |
| | | | 200° C. | |
| Oxalate | Form II | 0.22 | No form change, | Amorphous brown |
| | | | remains predominantly | solid |
| | | | crystalline | |
| Tosylate (1 equiv.) | Form I | 0.104 | No form change, | No form change, |
| | | | remains predominantly | partially crystalline |
| | | | crystalline | |
| Tosylate (1 equiv.) | Form II | 0.422 | Gum formed | Possible form change, |
| | | | | partially crystalline |
| Tosylate (2 equiv.) | Form I | 0.089 | No form change, | No form change, |
| | | | remains predominantly | remains |
| | | | crystalline | predominantly |
| | | | | crystalline |
| Tosylate (2 equiv.) | Form II | 0.303 | Gum formed | Possible form change, |
| | | | | partially crystalline |
| 1,2-Ethane disulfonic | Form I | 0.21 | Significant loss in | Significant loss in |
| acid (1 eq.) | | | crystallinity | crystallinity |
| 1,2-Ethane disulfonic | Form II | 0.62 | No form change but | Significant loss in |
| acid (1 eq.) | | | some loss in crystallinity | crystallinity |
| 1,2-Ethane disulfonic | Form I | 0.34 | No form change, | No form change, |
| acid (2 eq.) | | | remains partially | remains partially |
| | | | crystalline. | crystalline. TGA |
| | | | | showed an initial |
| | | | | weight loss of 6.28% |
| | | | | which could indicate |
| | | | | potential solvation |
| | | | | (8.4% required for 1 |
| | | | | equiv. of MEK). |
| | | | | No other weight |
| | | | | losses prior to |
| | | | | degradation. |
| 1,2-Ethane disulfonic | Form II | 0.25 | XRPD predominantly | XRPD predominantly |
| acid (2 eq.) | | | similar to input material. | similar to input |
| | | | | material. Partially |
| | | | | crystalline. TGA |
| | | | | showed an initial |
| | | | | weight loss of 3.7%, |
| | | | | which could indicate |
| | | | | potential hydration or |
| | | | | hygroscopic material |
| | | | | (monohydrate would |
| | | | | have 2.23% water). |
| | | | | No other weight |
| | | | | losses prior to |
| | | | | degradation. |
| HBr (1 eq.) | Form I | 1.27 | No form change, slight | No Form change. |
| (Form I for both 1 | | | loss in crystallinity. | Slight loss in |
| and 2 equiv. was the | | | | crystallinity. |
| same form) | | | | TGA showed an |
| | | | | initial weight loss of |
| | | | | 0.83% probably due |
| | | | | to unbound volatiles. |
| | | | | No other weight |
| | | | | losses prior to |
| | | | | degradation. |
| HBr (2 eq.) | Form II | 2.38 | Changed to Form I, | Potential new form, |
| | | | poorly crystalline. | but poorly crystalline |
| Naphthalene-2- | Form I | 0.34 | No form change, small | No form change, |
| sulfonic | | | loss in crystallinity | small loss in |
| acid (1 eq.) | | | | crystallinity |
| | | | | TGA showed an |
| | | | | initial weight loss of |
| | | | | 1.86% probably due |
| | | | | to unbound volatiles. |
| | | | | No other weight |
| | | | | losses prior to |
| | | | | degradation. |
| Naphthalene-2- | Form I | 0.87 | No form change, poor | No form change, poor |
| sulfonic | | | crystallinity. | crystallinity |
| acid (2 eq.) | | | | |
| Naphthalene-2- | Form II | 0.86 | No form change, | No form change, but |
| sulfonic | | | remains partially | loss of crystallinity |
| acid (2 eq.) | | | crystalline. | |
| | | | TGA showed an initial | |
| | | | weight loss of 0.71% | |
| | | | probably due to unbound | |
| | | | volatiles and a second | |
| | | | loss of 1.61% associated | |
| | | | with the melt (ca. | |
| | | | 138° C.) which could | |
| | | | indicate some bound | |
| | | | water or solvent | |
| | | | (monohydrate would | |
| | | | lave 1.82 wt % water). | |
| Naphthalene-2- | Form III | 0.74 | Converted to Form II, | Converted to Form II, |
| sulfonic | | | but partially crystalline. | but partially |
| acid (2 eq.) | | | | crystalline. |
| | | | | TGA showed an |
| | | | | initial weigh loss of |
| | | | | 1.41% probably due |
| | | | | to unbound volatiles |
| | | | | and a second loss of |
| | | | | 1.32% associated with |
| | | | | the melt (ca. 135° C.). |
| | | | | Could indicate some |
| | | | | bound water or |
| | | | | solvent. |
| Naphthalene-2- | Form IV | 0.76 | Converted to Form II, | Converted to Form II, |
| sulfonic | | | but poorly crystalline. | but poorly crystalline. |
| acid (2 eq.) | | | | |
| 1,5 Naphthalene | Form I | Below LOQ | No form change, | Possible form change, |
| disulfonic | | | remains partially | but poorly crystalline. |
| acid (1 eq.) | | | crystalline. TGA | |
| | | | showed an initial weight | |
| | | | loss of 2.94% probably | |
| | | | due to unbound volatiles | |
| | | | and another weight loss | |
| | | | of 6.00%, which could | |
| | | | indicate potential | |
| | | | salvation (mono | |
| | | | acetonitrile solvate | |
| | | | would have ca. | |
| | | | 4.29 wt %). No other | |
| | | | weight loss prior to | |
| | | | degradation. | |
| 1,5 Naphthalene | Form I | Below LOQ | No form change, but | Predominantly |
| disulfonic | | | loss in crystallinity. | amorphous. |
| acid (2 eq.) | | | | |
| Camphor-10-sulfonic | Form I | 1.05 | No form change, slight | No form change, |
| acid (1 eq.) | | | loss in crystallinity | slight loss in |
| | | | TGA showed an initial | crystallinity |
| | | | weight loss of 2.13% | |
| | | | probably due to unbound | |
| | | | volatiles. No other | |
| | | | weight loss prior to | |
| | | | degradation. | |
| Camphor-10-sulfonic | Form II | 0.58 | Mixture of Form I and | Converted to Form I, |
| acid (1 eq.) | | | Form II. Some loss in | partially crystalline. |
| | | | crystallinity. TGA | |
| | | | showed a 3.3% weight | |
| | | | loss between 25-120° C. | |
| | | | Possible bound and | |
| | | | unbound water/solvent | |
| | | | present. No other weight | |
| | | | losses prior to | |
| | | | degradation. | |
| Camphor-10-sulfonic | Form I | 0.89 | Amorphous | Amorphous |
| acid (2 eq.) | | | | |

From these results the bis-besylate salt was selected to be scaled up, using acetone as the solvent. In addition, the hydrobromide salt was selected to be scaled up, using acetonitrile:water (90:10) as the solvent. The mono-maleate and bis-hydrochloride salts were also selected for scale-up experiments to assess whether these are solvated/hydrated.

### Example 3

### Secondary Screen of Bis-Besylate Salt

Approximately 5 mL of acetone was added to approximately 800 mg of Compound 1 to form a slurry. In a separate vial, approximately 3 mL acetone was added to 2 equivalents of benzenesulfonic acid to dissolve the acid. The acid solution was then added in small aliquots to the free base slurry while stirring. After the complete addition of the acid, a gum/oil-like material initially formed, however, this converted to a solid after ca. 30 minutes of stirring. The reaction was stirred for ca. 1.5 days before being isolated and dried. The material was initially dried at ambient under vacuum (ca. 22° C.) for 3 days, however, approximately 6.7% acetone was still present at this stage. A portion was then dried for a further 2 days at 40° C. under vacuum after which ca. 2.7% acetone remained. The material was then dried for a further 2 days at 60° C. under vacuum. The yield was 1.1 g of material (86%).

To examine whether the citric acid in the buffers was having an effect on the solubility values obtained for pH 3, 4.5 and 6.6, the thermodynamic solubility experiments were repeated at these pH values using KHP/HCl for pH 3, KHP/NaOH for pH 4.5 and phosphate/NaOH for pH 6.6. The remaining solids were also analysed by XRPD analysis to establish if any changes in the solid form occurred.

XRPD analysis (FIG. 1) showed the material to be crystalline. The diffractogram is consistent with the small scale bis-besylate Form I diffractograms obtained during the primary salt screen.

TGA/DTA was carried after 3 days of drying at ambient under vacuum as well as after further drying for 2 days at 40° C. under vacuum and 2 days at 60° C. under vacuum. After the ambient drying process, the TGA showed a 6.7% weight loss between ca. 50-150° C. (FIG. 2) (for an acetone solvate, 1 mole equivalent of acetone would be ca. 6.3 wt %). After further drying, the TGA showed a 0.47% weight loss from the outset, likely due to unbound moisture or solvent. A further small 0.16% weight loss corresponded with the endotherm at onset ca. 142° C. (FIG. 3).

DSC analysis (FIG. 4) indicated a broad endotherm from the outset likely due to unbound solvent. A second endotherm was present at onset ca. 139.4° C. (peak 146.1° C.).

Polarised Light Microscopy (not shown) showed birefringent particles with no clearly defined morphology present.

IR spectroscopy (FIG. 5) showed a number of differences and shifts in comparison with the freebase and benzenesulfonic acid.

¹H NMR spectroscopy (FIG. 6) indicated that a number of the Compound 1 and benzenesulfonic acid peaks appear to be overlapping, however, the stoichiometry is approximately 2:1 benzenesulfonic acid: Compound 1. The acetone present does not appear to be a stoichiometric amount.

DVS analysis (FIG. 7) showed a water uptake of ca. 2.2% between 20 and 70% RH. The difference between the mass of the first sorption cycle and the desorption and second sorption cycle at 20% RH is likely due to the loss of excess acetone in the first cycle. The material also appears to hydrate during DVS analysis as indicated by the change in polymorphic form seen by post DVS XRPD analysis (not shown). The XRPD diffractogram also showed some loss in crystallinity.

Karl Fischer Coulometry indicated a ca. 0.77% water content (Note: due to the manual introduction of the solid material into the titration cell, measured values below 1% are generally slightly higher than the actual water content).

The HPLC purity evaluation (not shown) indicated a purity of ca. 97.6% for the bis-besylate salt with the main peak eluting at a retention time of ca. 13.05 minutes.

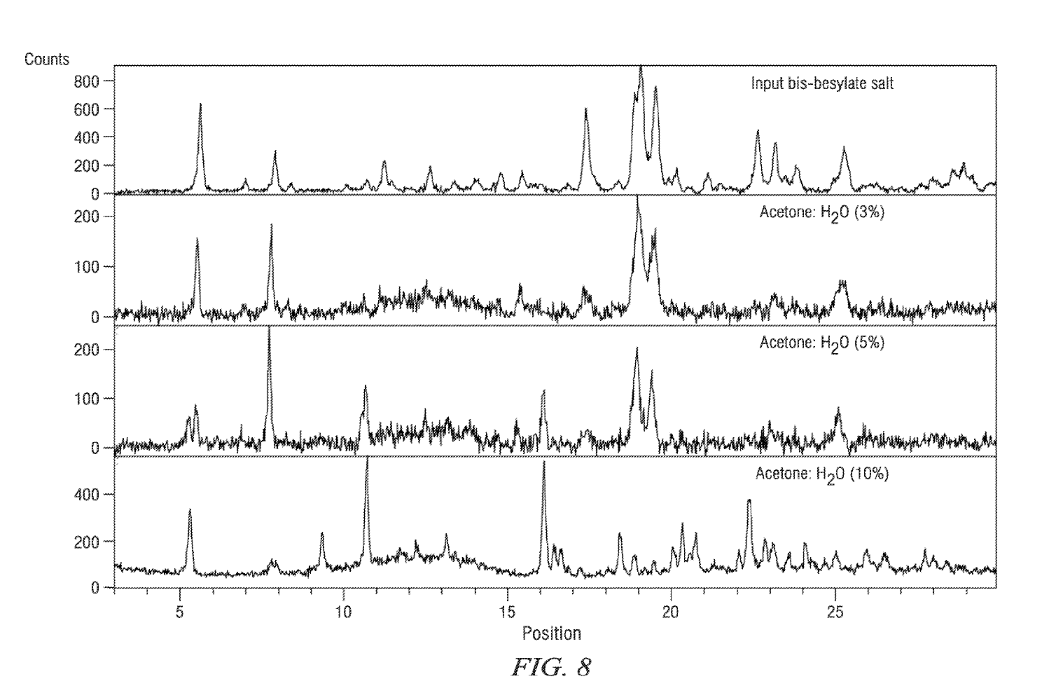

Slurries of the bis-besylate salt were created in acetone: water mixtures (3%, 5% and 10%) and stirred at ambient for ca. 4 days. The resulting solids were then analyzed by XRPD to determine if any changes had occurred on slurrying. The hydration study results from XRPD analysis (FIG. 8) are summarised in Table 3.

TABLE 3
Hydration Study Results

| Solvent System | Result of slurrying |
|---|---|
| Acetone:water (3%) | Corresponds with the input bis- |
| | besylate salt material. |
| Acetone:water (5%) | Appears to be a mixture of the input |
| | material and a possible hydrate. |
| Acetone:water (10%) | Different from the bis-besylate input |
| | material, likely hydrated. Peaks |
| | correspond with post DVS XRPD |
| | peaks. |

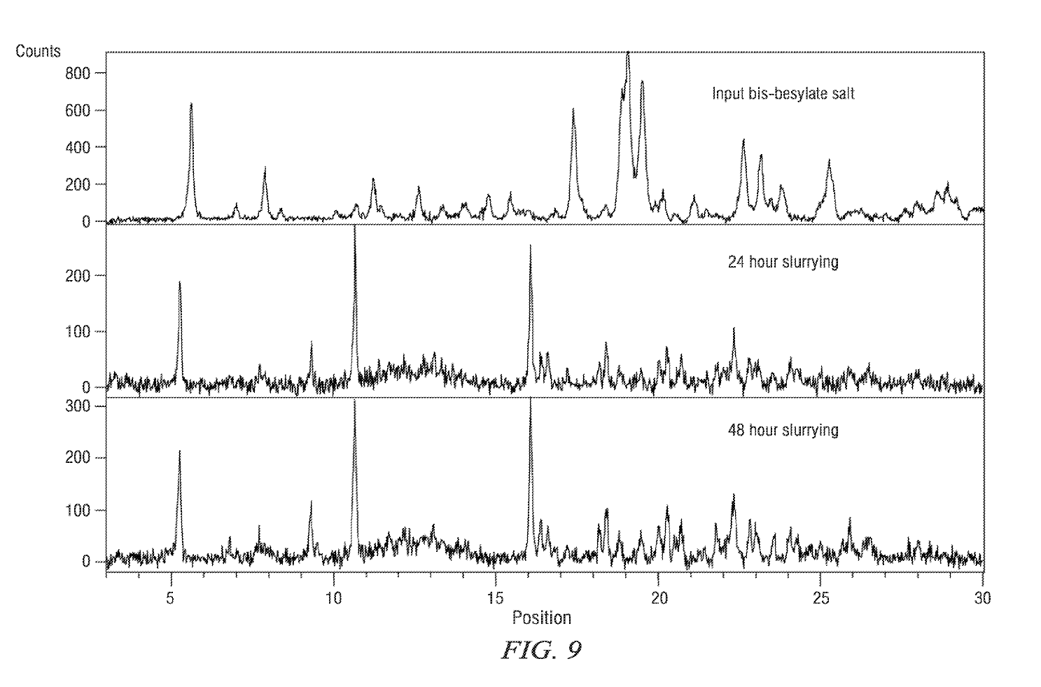

The bis-besylate salt was slurried in deionized water at ambient temperature (ca. 22° C.). A sample of solid was taken at 24 & 48 hours and analysed by XRPD. The pH of the supernatant was also monitored. The Salt Disproportionation study results from XRPD analysis (FIG. 9) are summarised in Table 4.

TABLE 4
Disproportionation Study Results

| Timepoint | Solvent System | Result of slurrying |
|---|---|---|
| 1 hr | pH 2-3 | Yellow gum present. |
| 24 hrs | pH 1-2 | Different from starting material, appears to |
| | | have hydrated (corresponds with |
| | | Acetone:water (90:10%) hydration sample). |
| 48 hrs | pH 1-2 | Different from starting material, appears to |
| | | have hydrated (corresponds with |
| | | Acetone:water (90:10%) hydration sample). |

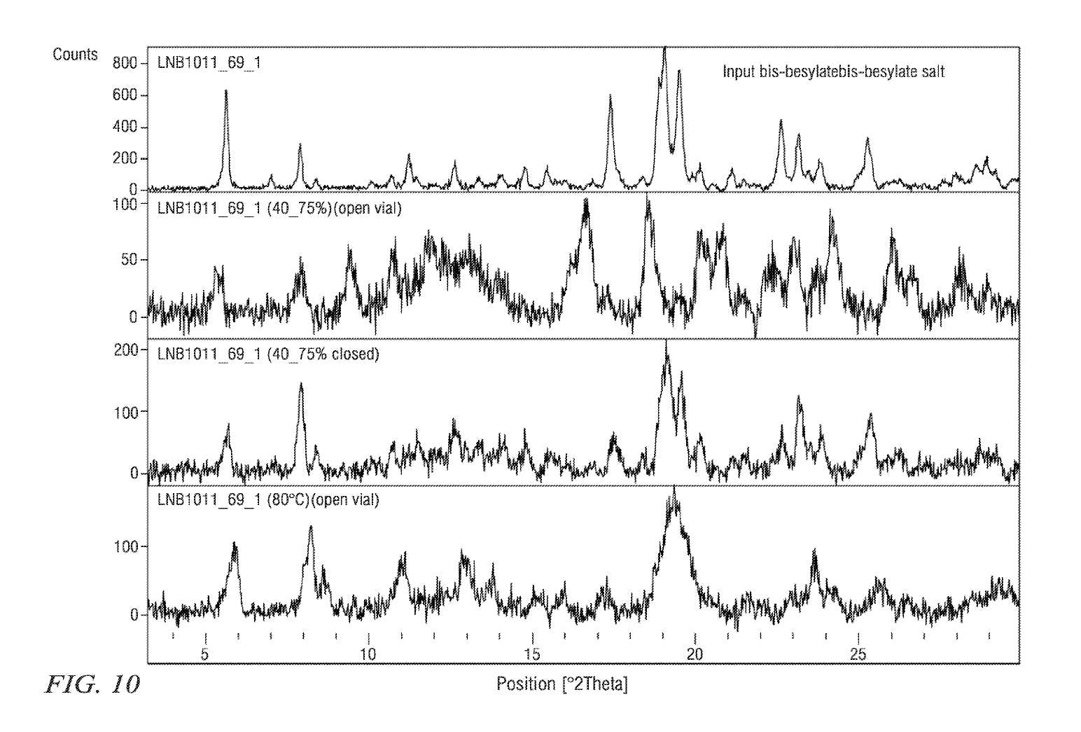

The bis-besylate salt was exposed to environments of 40° C./75% RH (relative humidity, open and closed vial) and 80° C. (open vial) for 1 week to determine stability. Resulting solids were analysed by XRPD and HPLC to establish if any changes had occurred. The 1 Week stability study results from XRPD (FIG. 10) and HPLC analysis (not shown) at 40° C./75% RH using an open and closed vial and 80° C. using an open vial are indicated Table 5.

TABLE 5
1 Week Stability Study Results

| Storage | | |
|---|---|---|
| Condition | HPLC | XRPD |
| 40° C./75% RH | 97.22% | Change in polymorphic form, likely due |
| (open vial) | | to hydration. |
| 40° C./75% RH | 97.35% | No change in polymorphic form. |
| (crimped vial) | | |
| 80° C. | 97.20% | Corresponds predominantly with input |
| (open vial) | | material, with loss in crystallinity. |

Slurries of the bis-besylate salt were created in media of various pH (pH 1; pH 3; pH 4.5 and pH 6.6) and shaken for ca. 24 hours. After 24 hours, the slurries were filtered and the solution analysed by HPLC in order to determine the solubility at the various pH levels. The remaining solids were also analysed by XRPD analysis to establish if any changes in the solid form occurred. For the buffer solutions, KCl/HCl was used for pH 1 and citrate/phosphate combinations for pH 3, 4.5 and 6.6. Thermodynamic solubility studies indicated the results shown in Table 6.

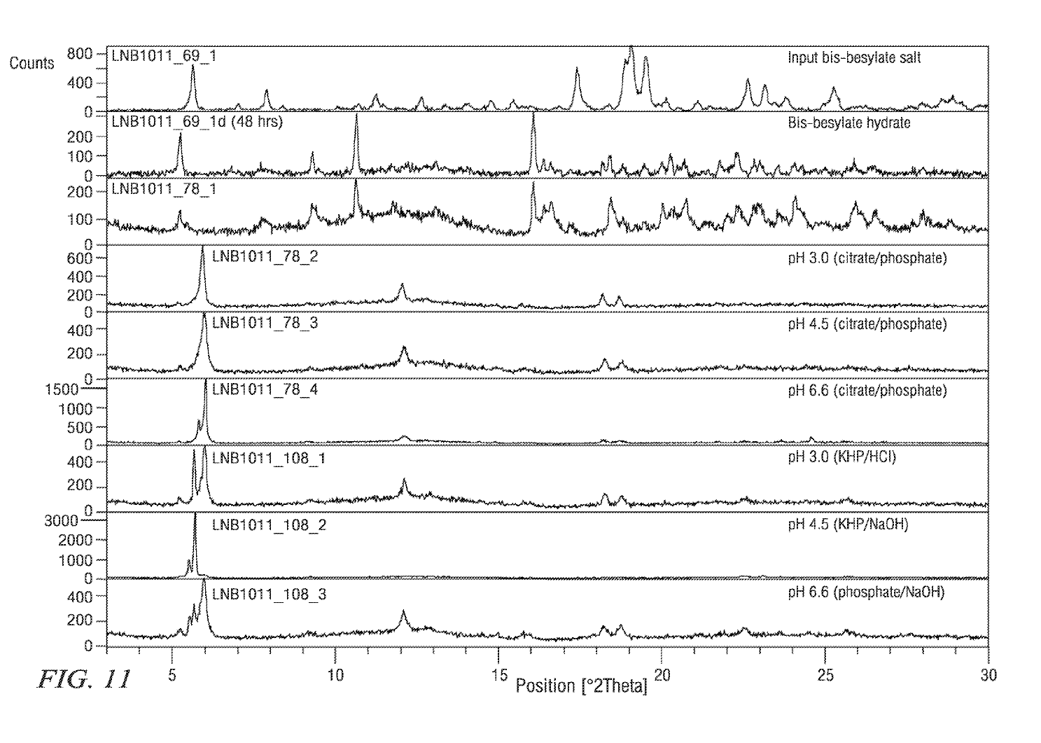

TABLE 6
Thermodynamic Solubility Results

| | Buffers | Solubility | |
|---|---|---|---|
| pH | used | (mg/ml) | XRPD of excess solids (FIG. 11) |
| pH 1.0 | KCl/HCl | 4.93 mg/ml | Diffractogram corresponds with the |
| | | | hydrated bis-besylate. |
| pH 3.0 | Citrate/ | 0.24 mg/ml | Change in diffractogram - does not |
| | Phosphate | | correspond with any known forms of |
| | | | the bis-besylate or Compound 1. |
| | | | Does not correspond with the |
| | | | solids used in the buffers. |
| pH 4.5 | Citrate/ | 0.43 mg/ml | Change in diffractogram - does not |
| | Phosphate | | correspond with any known forms of |
| | | | the bis-besylate or Compound 1. |
| | | | Does not correspond with the |
| | | | solids used in the buffers. |
| pH 6.6 | Citrate/ | 0.66 mg/ml | Change in diffractogram - does not |
| | Phosphate | | correspond with any known forms of |
| | | | the bis-besylate or Compound 1. |
| | | | Does not correspond with the |
| | | | solids used in the buffers. |
| pH 3.0 | KHP/HCl | 0.26 mg/ml | Change in diffractogram - does not |
| | | | correspond with any known forms of |
| | | | the bis-besylate or |
| | | | Compound 1. Does not correspond |
| | | | with the solids used in the buffers. |
| pH 4.5 | KHP/ | 0.10 mg/ml | Change in diffractogram - does not |
| | NaOH | | correspond with any known forms of |
| | | | the bis-besylate or Compound 1. |
| | | | Does not correspond with the solids |
| | | | used in the buffers. |
| pH 6.6 | Phosphate/ | 0.17 mg/ml | Change in diffractogram - does not |
| | NaOH | | correspond with any known forms of |
| | | | the bis-besylate or Compound 1. |
| | | | Does not correspond with the solids |
| | | | used in the buffers. |

When initially setting up the slurries for thermodynamic solubility determinations, gums were obtained in all of the pH media used, however, upon shaking, the gums converted to solids after ca. 2 hours. The XRPD analysis of the excess solid from the slurries after the solubility experiments, indicate that for pH 1, the bis-besylate salt hydrates while slurrying. Hence, the solubility value obtained is likely an indication of the solubility of the hydrated material. The diffractograms for the remaining samples appear different from the input material as well as all identified forms of the bis-besylate and Compound 1 free base. The diffractograms also appear different from the diffractograms of the solids used to make up the buffers. The solubility values obtained using these pH buffers are likely not representative of the bis-besylate salt which was initially placed into the solutions.

Approximately 100-120 mg of each form was compressed into discs by placing the material into a die (diameter: 13 mm) and compressing the die under 5 tons of pressure in a hydraulic press for ca. 2 minutes. A Sotax AT7 (conformed to EP2 and USP2) dissolution instrument was used containing paddles to stir the media at 100 rpm. Dissolution media of pH 3 (1% SDS) and pH 4.5 (1% SDS) were prepared using citrate/phosphate buffer. All materials were tested in 750 ml of the buffer medium. Discs were added at time=0 seconds and allowed to sink to the bottom of the dissolution vessel before stirring began. ca. 1 ml aliquots of media were extracted from the dissolution vessels at times 1, 5, 10, 15, 30, 60, 120, 240 minutes and 24 hours, and tested for dissolved salt concentration by HPLC-UV. The dissolution tests were carried out in duplicate. For both dissolution media, the peak areas for the initial time points (up to 15 minutes), fell below the limit of quantification, however, when plotting Dissolution rate vs. time, the steepest part of the curve occurs during these early time points.

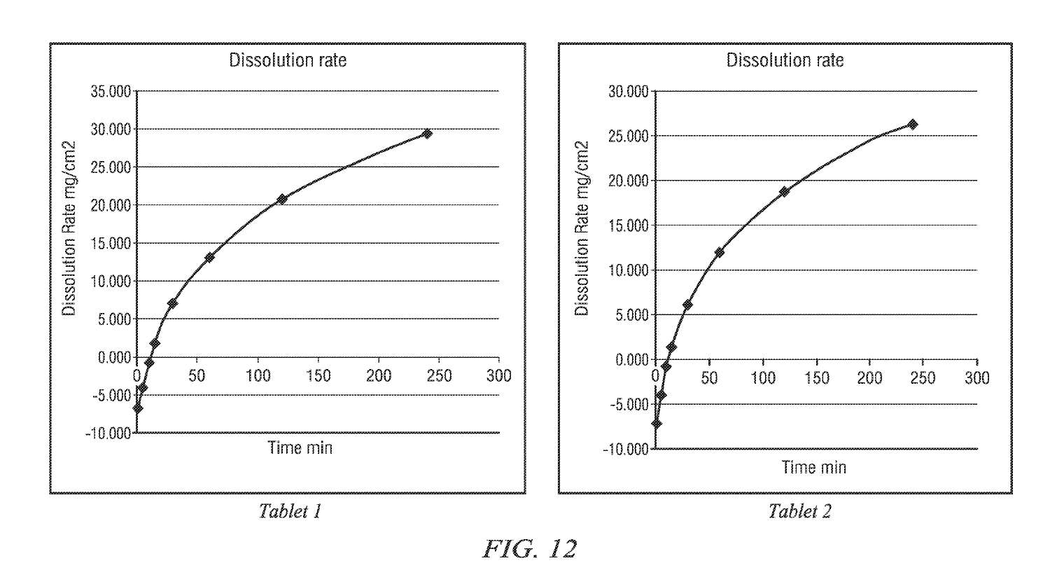

For pH 4.5, when plotting the curve of Dissolution rate vs. Time (FIG. 12), the intrinsic dissolution values obtained from the early time points on the curve (steepest part of the curve) were approximately 0.61 mg/cm²/min for both tablets 1 and 2. At the later time points, intrinsic dissolution values of 0.09 mg/cm²/min and 0.08 mg/cm²/min were obtained for tablets 1 and 2, respectively.

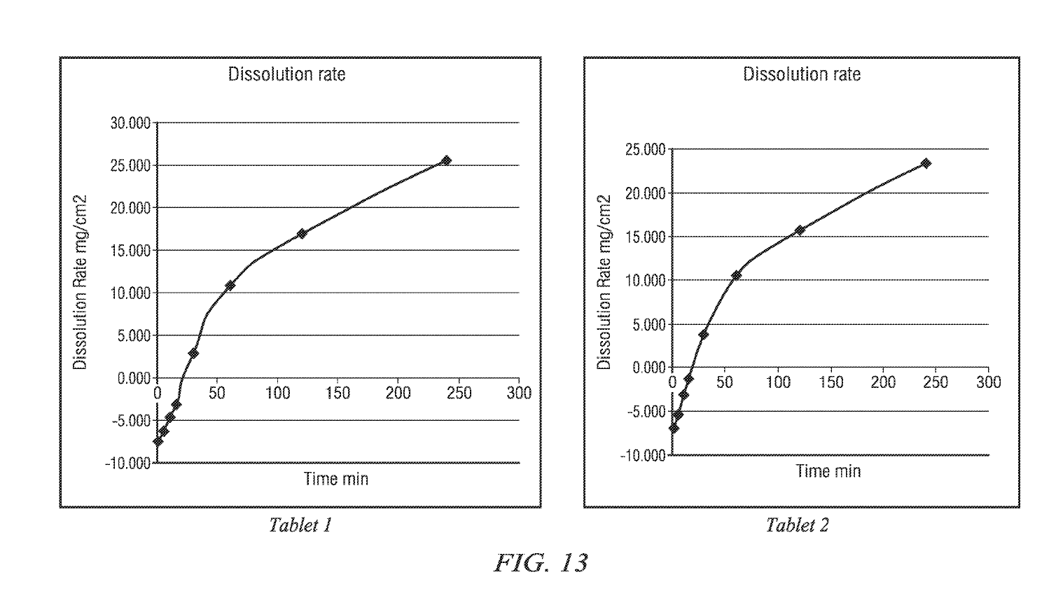

For pH 3.0, when plotting the curve of Dissolution rate vs. Time (FIG. 13), the intrinsic dissolution values obtained from the early time points on the curve (steepest part of the curve) were approximately 0.36 mg/cm²/min for tablet 1 and 0.38 mg/cm²/min for tablet 2. At the later time points, intrinsic dissolution values of 0.08 mg/cm²/min and 0.07 mg/cm²/min were obtained for tablets 1 and 2, respectively.

### Example 4

### Secondary Screen of Bis-Besylate Hydrate Salt

Approximately 3 mL of acetone was added to ca. 500 mg of Compound 1 to form a slurry. In a separate vial, ca. 1 mL of acetone was added to 2 equivalents of benzenesulfonic acid in order to dissolve the acid. The acid solution was then added in small aliquots to the freebase slurry while stirring. The reaction was stirred for ca. 1 day while temperature cycling between 0 and ambient temperature (ca. 22° C.). After 1 day, deionized water was added to the reaction mixture and the slurry was allowed to stir for ca. 3 hours before being isolated and dried at ambient under vacuum.

XRPD analysis (FIG. 14) showed the material to be crystalline. The diffractogram is consistent with the bis-besylate hydrate obtained from the hydration studies of the bis-besylate salt.

TGA/DTA indicated a weight loss of ca. 2.1% between ca. 70-100° C. (FIG. 15). This corresponds approximately with the 2.03 wt % water required for a monohydrate. A ca. 2.2% weight loss was present from the outset to ca. 70° C., likely due to unbound water. Although the total ca. 4.2% weight loss corresponds approximately with a dihydrate, the first weight loss occurs from the outset followed by a second clear weight loss corresponding with mono amounts of water. As the first weight loss occurs from ca. 25° C., this would likely be due to unbound water.

DSC analysis indicated a broad endotherm between ca. 40-115° C. Two further endotherms were then present at onset 119.7° C. (peak 134.3° C.) and onset 153.8° C. (peak 165.1° C.) (FIG. 16).

PLM analysis showed some birefringence, however the particle size is very small and no clear morphology could be seen (not shown). Hot-stage microscopy was carried out on a sample of the bis-besylate hydrate. No visual changes could be observed prior to the material melting and degrading (turned brown) at ca. 160° C.

IR analysis (FIG. 17) showed differences from both the free base and benzenesulfonic acid spectra as well as some differences when comparing the spectra of the input bis-besylate salt with that of the hydrated material.

¹H NMR spectroscopy (FIG. 18) indicated that a number of the Compound 1 and benzenesulfonic acid peaks appear to be overlapping, however, the stoichiometry appears to be approximately 2:1 benzenesulfonic acid: Compound 1. A small non-stoichiometric amount of acetone was present in the spectrum.

DVS analysis (FIG. 19) showed a water uptake of ca. 1.3% between 20 and 70% RH. No hysteresis was seen between the sorption and desorption cycles. The XRPD diffractogram of the material post DVS analysis was consistent with the diffractogram of the input bis-besylate hydrate material (not shown).

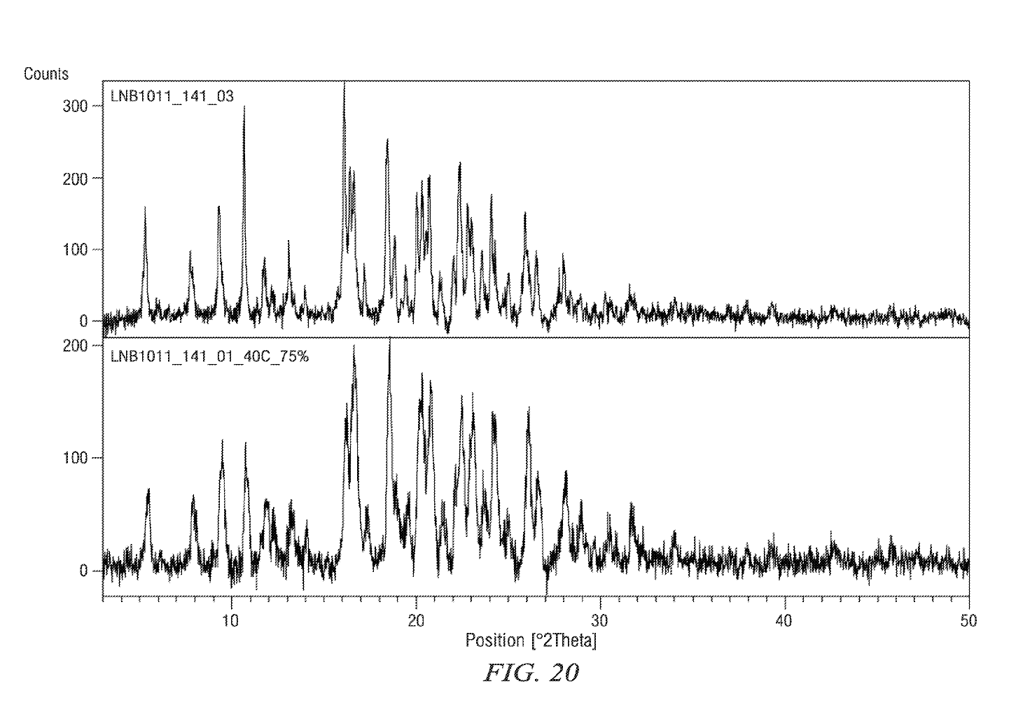

The 1 week stability data at 40° C./75% RH (open container) indicated that by XRPD, the material remained consistent with the input material with no changes in polymorphic form (FIG. 20).

HPLC purity determinations indicated an initial purity of ca. 98.4% and a purity of ca. 98.3% after 1 week storage at 40° C./75% RH.

Thermodynamic solubility studies of the bis-besylate hydrate indicated the results shown in Table 7.

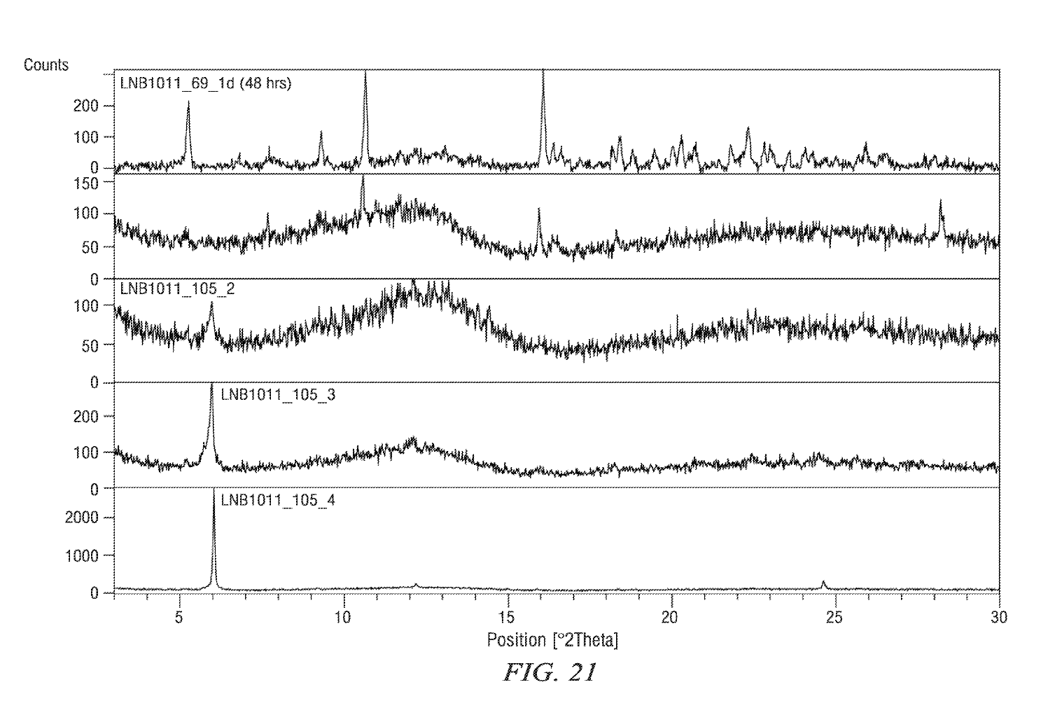

TABLE 7
Thermodynamic Solubility Results

| | | Solubility | |
|---|---|---|---|
| pH | Buffers | (mg/ml) | XRPD of excess solids (FIG. 21) |
| pH 1.0 | KCl/HCl | 4.39 mg/ml | Very little solid present for XRPD, |
| | | | however the peaks which are visible |
| | | | in the diffractogram appear to |
| | | | correspond with the hydrated bis- |
| | | | besylate diffractogram. |
| pH 3.0 | Citrate/ | 0.016 mg/ml | Very little solid present for XRPD, |
| | Phosphate | | however, a change in the |
| | | | diffractogram is seen where it does |
| | | | not correspond with any identified |
| | | | forms of the bis-besylate salt or |
| | | | Compound 1. It also does not |
| | | | correspond with the solids used in |
| | | | the buffers. |
| pH 4.5 | Citrate/ | Below | Very little solid present for XRPD, |
| | Phosphate | LOQ | however, a change in the |
| | | | diffractogram is seen where it does |
| | | | not correspond with any identified |
| | | | forms of the bis-besylate salt or |
| | | | Compound 1. It also does not |
| | | | correspond with the solids used in |
| | | | the buffers. |
| pH 6.6 | Citrate/ | Not | Very little solid present for XRPD, |
| | Phosphate | detected by | however, a change in the |
| | | HPLC | diffractogram is seen where it does |
| | | | not correspond with any identified |
| | | | forms of the bis-besylate salt or |
| | | | Compound 1. It also does not |
| | | | correspond with the solids used in |
| | | | the buffers. |

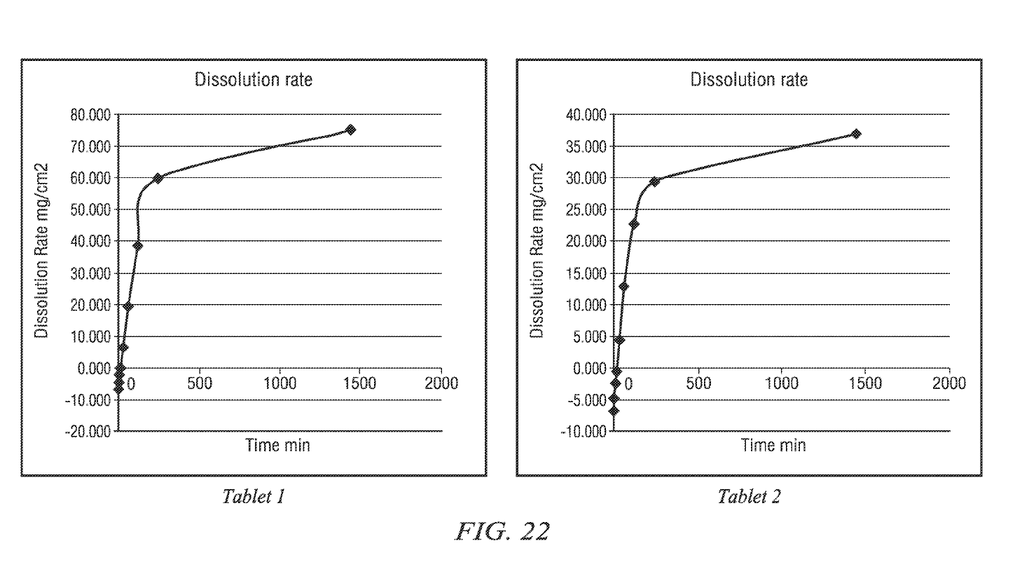

Intrinsic dissolution tests were carried out using pH 4.5 (1% SDS) and pH 3.0 (1% SDS). For both dissolution media, the peak areas for the initial time points (up to 15 minutes), fell below the limit of quantification, however when plotting Dissolution rate vs. time, the steepest part of the curve occurs during these early time points. For pH 4.5, when plotting the curve of Dissolution rate vs. Time (FIG. 22), the intrinsic dissolution values obtained from the early time points on the curve (steepest part of the curve) were approximately 0.43 mg/cm²/min for tablet 1 and 0.44 mg/cm²/min for tablet 2. At the later time points (toward the end of the dissolution study), intrinsic dissolution values of 0.012 mg/cm²/min and 0.006 mg/cm²/min were obtained for tablets 1 and 2, respectively.

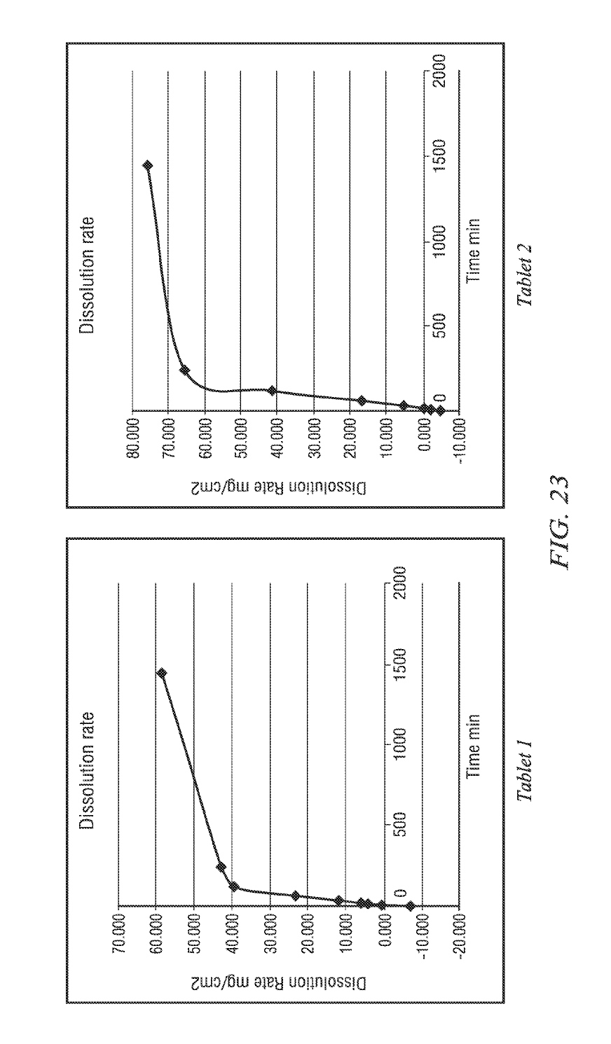

For pH 3.0, when plotting the curve of Dissolution rate vs. Time (FIG. 23), the intrinsic dissolution values obtained from the early time points on the curve (steepest part of the curve) were approximately 0.38 mg/cm²/min for tablet 1 and 0.39 mg/cm²/min for tablet 2. At the later time points, intrinsic dissolution values of 0.01 mg/cm²/min for both tablets 1 and 2 were obtained.

A larger batch of the bis-besylate hydrate salt was prepared using the following procedure. Approximately 20 mL of acetone was added to ca. 14 g of Compound 1 in a round bottomed flask to form a slurry. In a separate flask, ca. 10 mL of acetone was added to 2 equivalents of benzenesulfonic acid in order to dissolve the acid. The acid solution was then added in small aliquots to the freebase slurry whilst stirring at ca. 0° C. The resulting slurry was then allowed to stir at ambient for ca. 2 hours. It was then placed at ca. 5° C. for 2 days before stirring for a further 3 hours at ambient temperature. The acetone was then removed and ca. 20 mL of water was added to the material. The slurry was temperature cycled (0° C.—ambient temperature (ca. 22° C.)) in 2 hour cycles for ca. 1 day. The solid was then isolated by filtration and allowed to dry at ambient conditions under vacuum before analysis. The drying was continued for ca. 10 days.

The properties of the material from this larger batch were similar to those described above. In addition to those properties, it was noted that when the bis-besylate hydrate was left on the bench for 2 hours and TGA was again carried out, the sample seemed to pick up the water to have a total ca. 4.5% weight loss in the final TGA. It does not appear to be possible to remove the remaining 2% of unbound water by drying as this is regained when exposed to ambient conditions. Also, KF titration determined the water content of the material to be ca. 3.97%. While the ca. 4 wt % water would correspond theoretically with a dihydrate, the weight loss in the TGA appears to start from the outset followed by a more clear second weight loss which corresponds approximately with 1 equivalent of water. The material likely shows some hygroscopicity resulting in the initial TGA weight loss.

### Example 5

### Secondary Screen of Mono-Maleate Salt

Approximately 3 mL of dichloromethane was added to ca. 200 mg of Compound 1 to form a slurry. In a separate vial, ca. 1 mL of dichloromethane was added to 1 equivalent of maleic acid in order to dissolve the acid. The acid solution was then added in small aliquots to the freebase slurry while stirring. The slurry obtained was yellow in color. The reaction was stirred for ca. 1.5 days between 0° C. and ambient temperature (ca. 22° C.) and remained at ca. 4° C. for a further 2 days before being isolated and dried at ambient. The material was dried at ambient temperature under vacuum (ca. 22° C.) for ca. 2 days.

XRPD analysis (FIG. 24) showed the material to be crystalline. The diffractogram is consistent with the small scale mono-maleate Form I diffractogram obtained during the primary salt screen.

TGA/DTA was carried after 2 days of drying at ambient under vacuum. The TGA showed a 0.4% weight loss from the outset, likely due to unbound moisture or solvent. A large 10.9% weight loss is associated with endothermic/exothermic events in the DTA between ca. 145-185° C., followed by further weight losses due to likely degradation (FIG. 25).

DSC analysis indicated an endotherm at onset 160.4° C. (peak 163.8° C.), directly followed by an exotherm, likely due to recrystallisation and then final degradation (FIG. 26).

¹H NMR spectroscopy (FIG. 27) indicated approximately 1:1 stoichiometry of Compound 1: maleic acid. Dichloromethane was not present in the spectrum. Therefore, the mono-maleate salt does not appear to be solvated.

### Example 6

### Secondary Screening of Bis-Hydrochloride Salt (Form I)

Approximately 1.5 mL of acetonitrile:H₂O (90:10) was added to ca. 200 mg of Compound 1 to form a slurry. In a separate vial, ca. 1 mL of acetonitrile:H₂O (90:10) was added to 2 equivalents of hydrochloric acid. The acid solution was then added in small aliquots to the freebase slurry while stirring. The reaction was stirred for ca. 1.5 days between 0° C. and ambient temperature (ca. 22° C.) and remained at ca. 4° C. for a further 2 days before being isolated and dried at ambient temperature. The material was dried at ambient temperature under vacuum (ca. 22° C.) for ca. 2 days.

XRPD analysis (FIG. 28) showed the material to be crystalline. The diffractogram is consistent with the small scale bis-hydrochloride Form I diffractogram obtained during the primary salt screen.

TGA/DTA was carried after 2 days of drying at ambient under vacuum. The TGA showed a 2.7% gradual weight loss from the outset to ca. 180° C. A further 4.3% weight loss is seen between ca. 180-210° C., which corresponds with an endotherm in the DTA trace (FIG. 29).

DSC analysis indicated a broad endotherm between ca. 30-160° C. A further endotherm is then present at onset 206.4° C. (peak 226.5° C.), directly followed by a smaller endotherm at peak 238.2° C. (FIG. 30).

Karl Fischer analysis showed ca. 3.3% water content (ca. 2.8% water required for a monohydrate).

¹H NMR spectroscopy (FIG. 31) indicated that the spectrum had shifted in comparison with Compound 1, indicating likely salt formation. No signs of degradation could be seen. The free base peak appears to be partially overlapping with the region for acetonitrile, however, no significant amounts of acetonitrile appear to be present.

### Example 7

### Secondary Screening of Hydrobromide Salt (1 equiv.)

Approximately ca. 5 mL of acetonitrile: water (10%) was added to ca. 1 g of Compound 1 free base to form a slurry. In a separate vial, ca. 3 mL of acetonitrile: water (10%) was added to 1 equivalent of hydrobromic acid (48%). The acid solution was then added dropwise over a 1 hour period to the free base slurry whilst stirring and maintaining a temperature between 0-5° C. After the complete addition of the acid, a further 3 mL of acetonitrile: water (10%) was added. The reaction was stirred for ca. 1 day before being isolated and dried under vacuum at ambient (ca. 22° C.). A yield of ca. 79% was obtained.

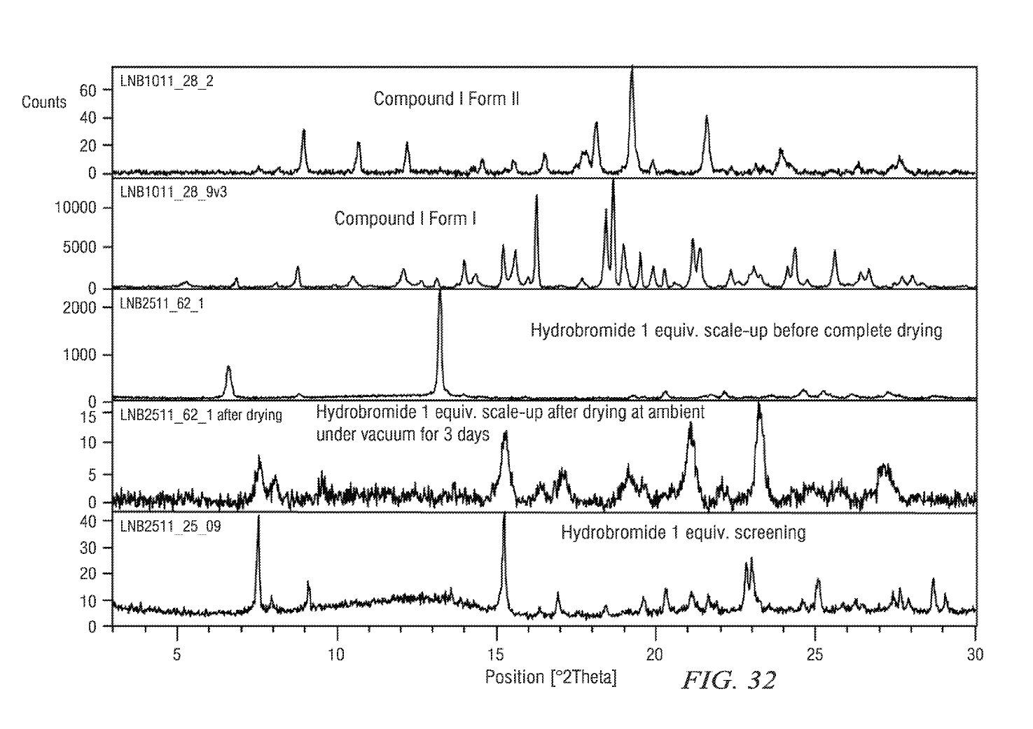

XRPD analysis (FIG. 32) was carried out on the wet sample and after drying. The analysis indicated that the material undergoes a form change upon drying. The diffractogram of the scaled up material, both before and after drying, was different from the diffractogram of the primary screen hydrobromide sample.

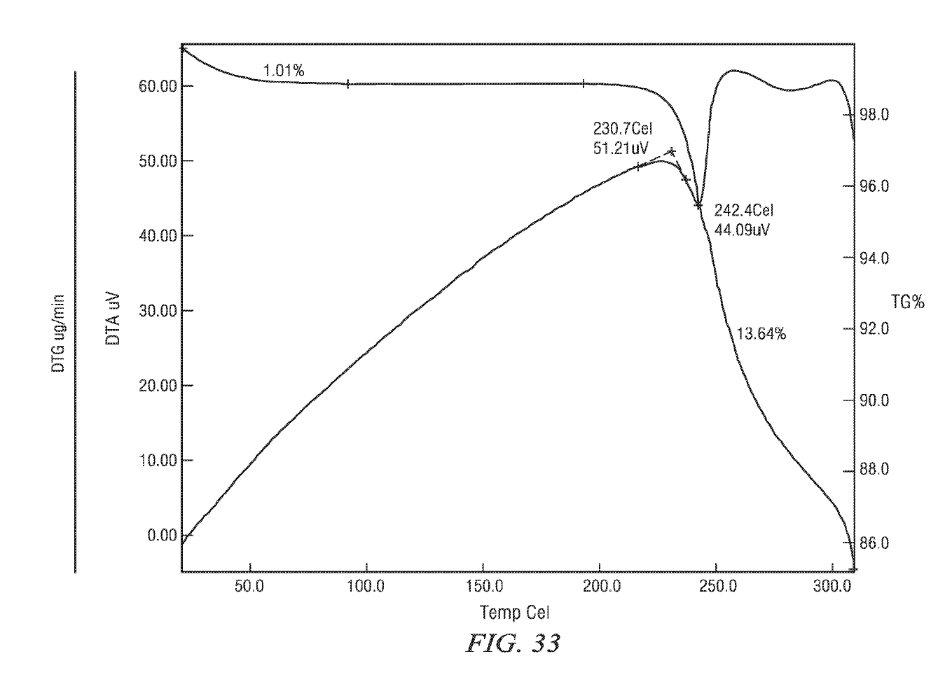

TGA/DTA showed a 1.01% weight loss from the outset, likely due to unbound moisture or solvent. No further weight losses were seen prior to degradation at onset ca. 230° C. (FIG. 33).

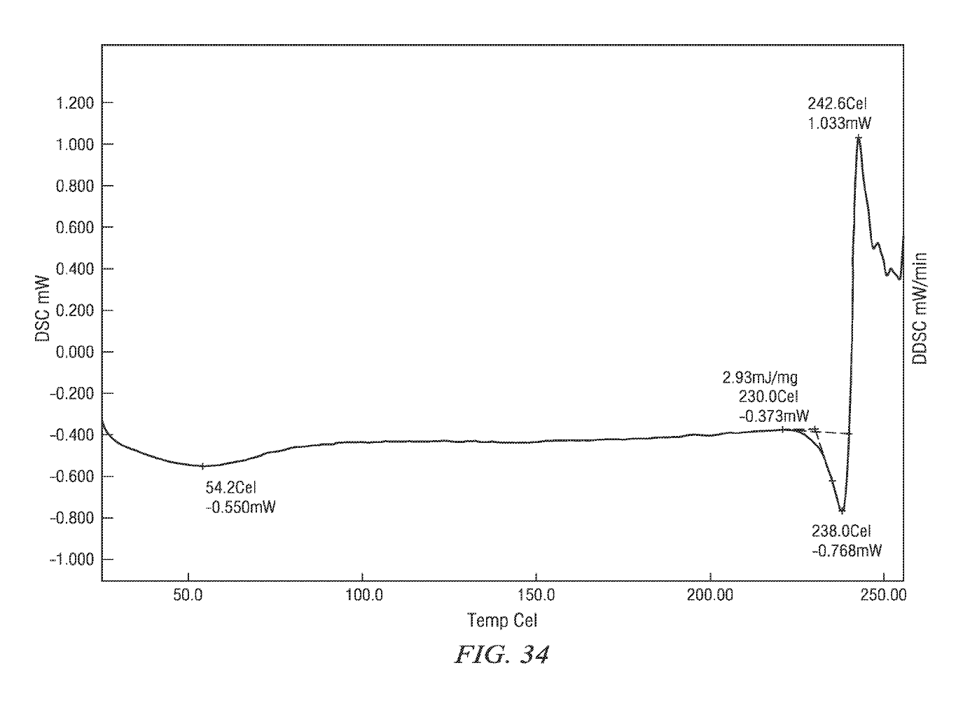

DSC analysis (FIG. 34) indicated a broad endotherm from the outset likely due to unbound solvent/water. A second endotherm was then seen at onset ca. 230° C. (peak 238° C.), followed by likely degradation.

Polarised Light Microscopy showed very small particles with no clearly defined morphology present (not shown).

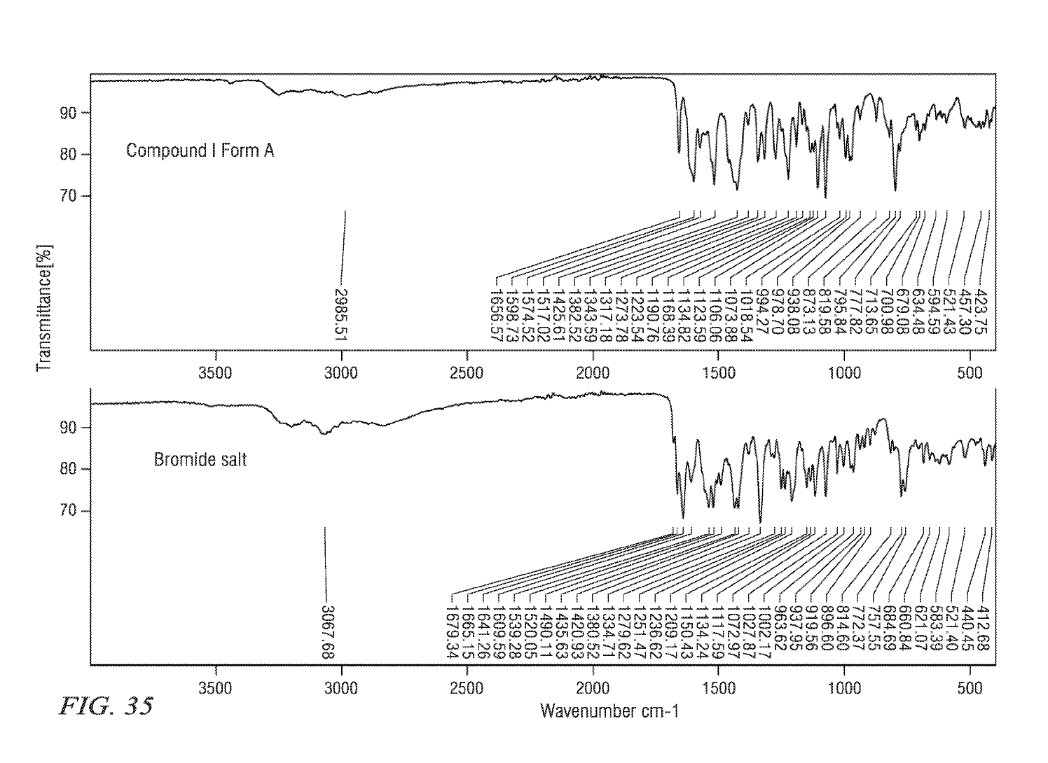

IR spectroscopy (FIG. 35) showed a number of differences and shifts in comparison with the freebase.

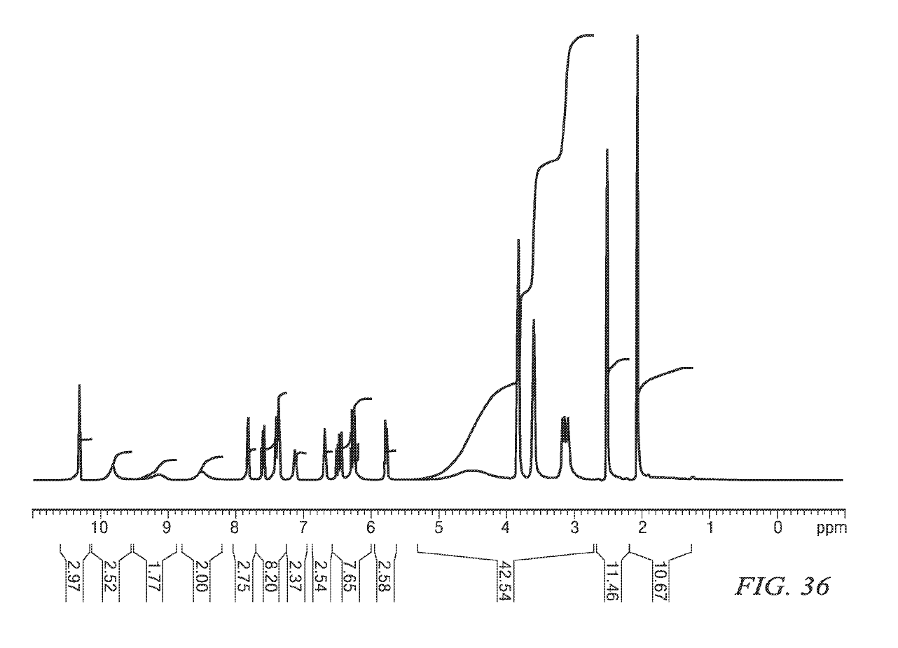

¹H NMR spectroscopy (FIG. 36) indicated a number of peak shifts in comparison with the freebase.

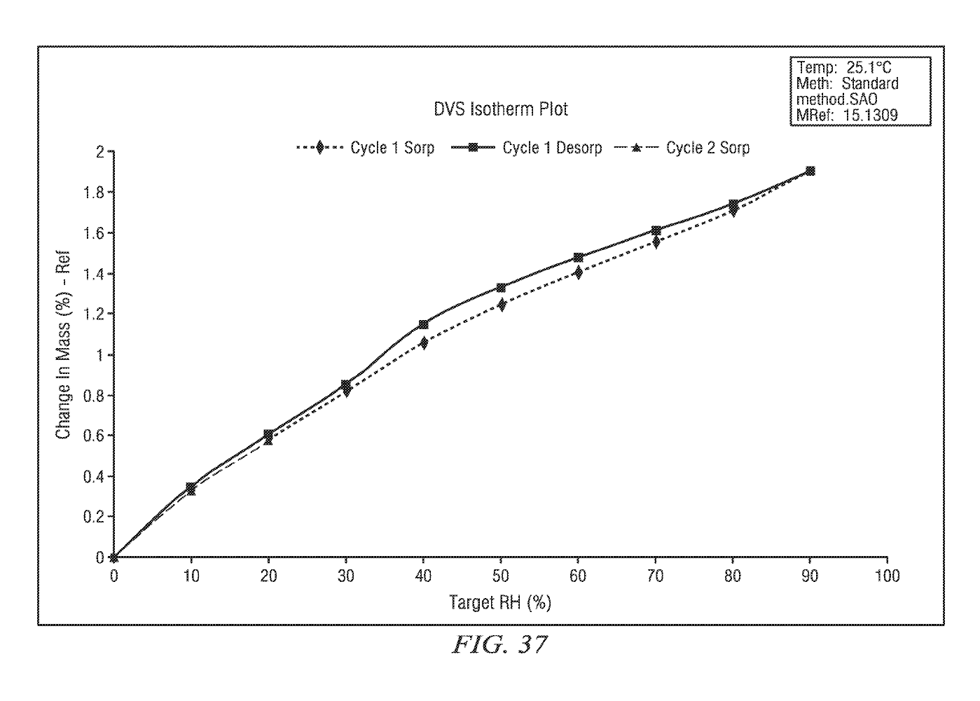

DVS analysis (FIG. 37) showed a water uptake of 0.97% between 20 and 70% RH. The water uptake between 0-90% RH is reversible showing very little hysteresis. Post DVS XRPD analysis indicated that the polymorphic form appears to remain consistent after exposure to varying RH % conditions (not shown).

Karl Fischer Coulometry indicated a ca. 1.65% water content.

The HPLC purity evaluation indicated a purity of ca. 97.5% for the hydrobromide salt with the main peak eluting at a retention time of ca. 13 minutes.

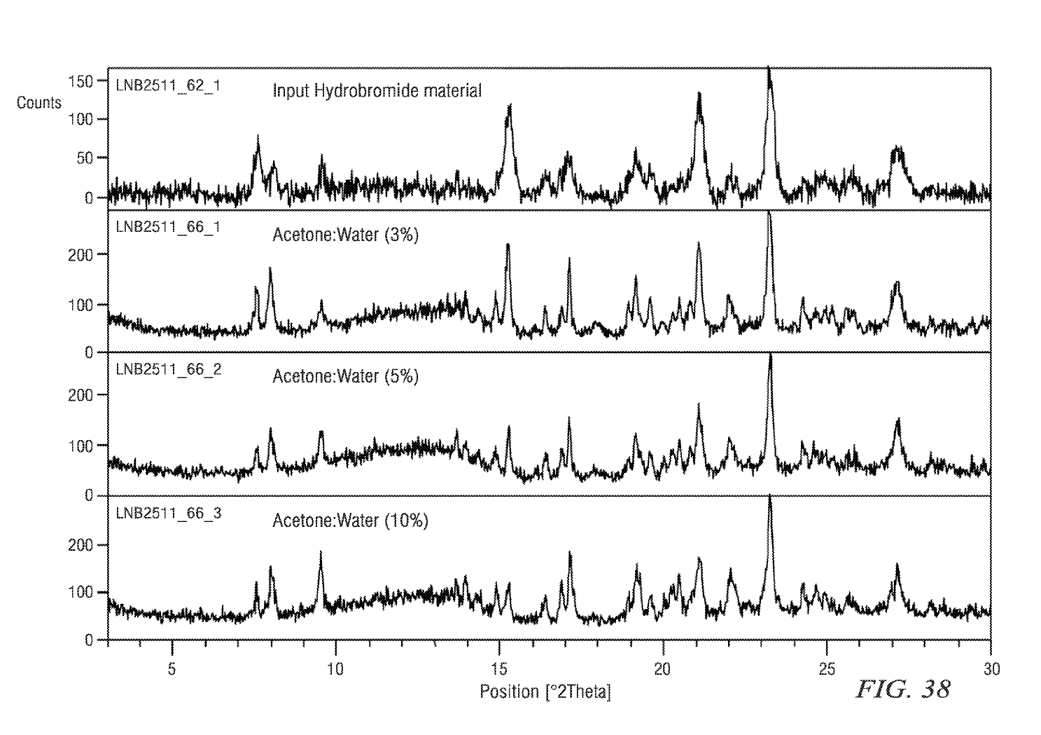

Slurries of the hydrobromide salt were created in acetone: water mixtures (3%, 5% and 10%) and stirred at ambient for ca. 3 days. The resulting solids were then analysed by XRPD to determine if any changes had occurred on slurrying. The hydration study results from XRPD analysis (FIG. 38) are summarised in Table 8.

TABLE 8
Hydration Study Results

| Solvent System | Result of slurrying |
|---|---|
| Acetone:water (3%) | Corresponds with the input hydrobromide form. |
| | Improvement in crystallinity. |
| Acetone:water (5%) | Corresponds with the input hydrobromide form. |
| | Improvement in crystallinity. |
| Acetone:water (10%) | Corresponds with the input hydrobromide form. |
| | Improvement in crystallinity. |

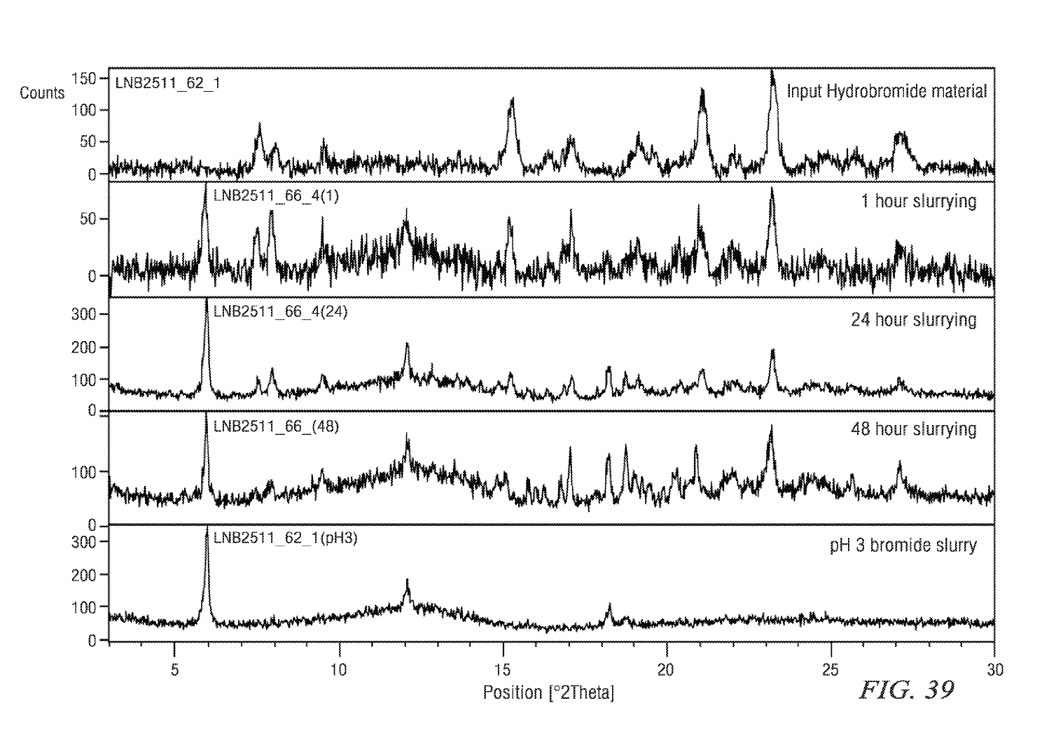

The hydrobromide salt was slurried in deionised water at ambient temperature (ca. 22° C.). A sample of solid was taken at 1, 24 & 48 hours and analysed by XRPD. The pH of the supernatant was also monitored. The Salt Disproportionation study results from XRPD analysis (FIG. 39) are summarised in Table 9.

TABLE 9
Disproportionation Study Results

| Time | | |
|---|---|---|
| point | pH | XRPD |
| 1 hr | pH 1 | The material appears to be a mixture of the input |
| | | hydrobromide material and a solid form that was |
| | | also obtained from slurrying the material in the |
| | | various pH solutions during thermodynamic solubility |
| | | studies. |
| 24 hrs | pH 1 | The material appears to be a mixture of the input |
| | | hydrobromide material and a solid form which was |
| | | obtained from slurrying the material in the various |
| | | pH solutions during thermodynamic solubility studies. |
| 48 hrs | pH 1 | The material appears to be a mixture of the input |
| | | hydrobromide material and a solid form which was |
| | | obtained from slurrying the material in the various |
| | | pH solutions during thermodynamic solubility studies. |

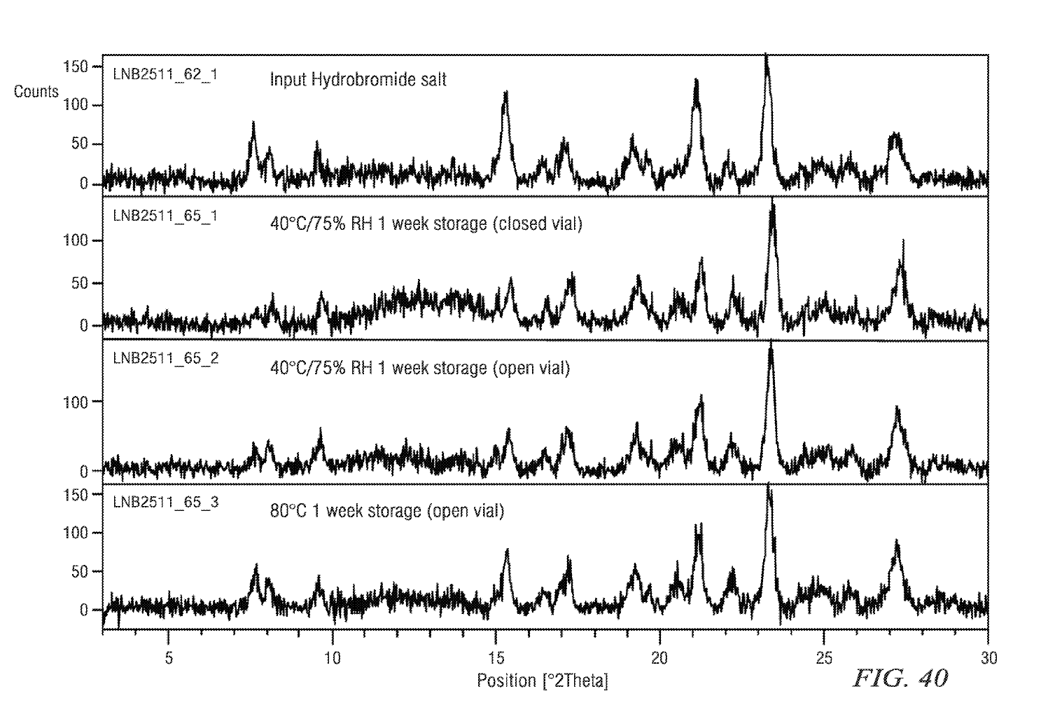

The hydrobromide salt was exposed to environments of 40° C./75% RH (open and closed vial) and 80° C. (open vial) for 1 week to determine stability. Resulting solids were analysed by XRPD and HPLC to establish if any changes had occurred. The 1 Week stability study results from XRPD (FIG. 40) and HPLC analysis at 40° C./75% RH using an open and closed vial and 80° C. using an open vial are indicated Table 10.

TABLE 10
1 Week Stability Study Results

| Condition | HPLC | XRPD |
|---|---|---|
| 40° C./75% RH | 97.2% | No polymorphic form changes observed |
| (closed vial) | | during storage. |
| 40° C./75% RH | 97.2% | No polymorphic form changes observed |
| (open vial) | | during storage. |
| 80° C. open vial | 97.1% | No polymorphic form changes observed |
| | | during storage. |

Slurries of the hydrobromide salt were created in media of various pH (pH 1; pH 3; pH 4.5 and pH 6.2) and shaken for ca. 24 hours. After 24 hours, the slurries were filtered and the solution analyzed by HPLC in order to determine the solubility at the various pH levels. The remaining solids were also analysed by XRPD analysis to establish if any changes in the solid form occurred. For the buffer solutions, KCl/HCl was used for pH 1 and citric acid/sodium citrate combinations for pH 3, 4.5 and 6.2. The thermodynamic solubility studies indicated the results shown in Table 11.

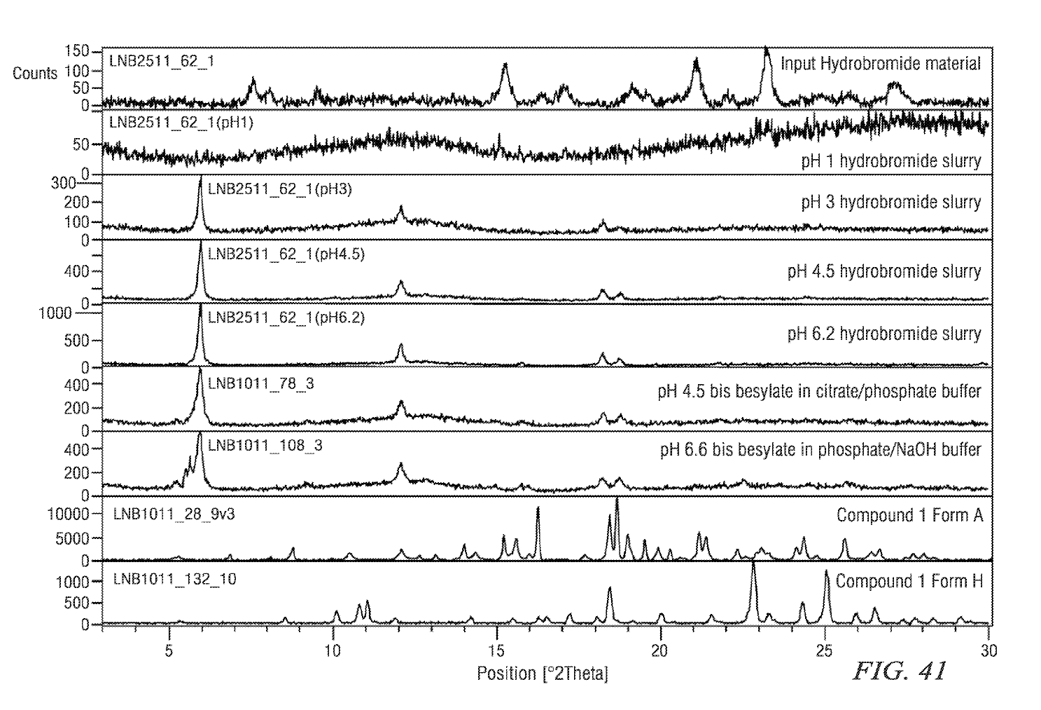

TABLE 11
Thermodynamic Solubility Results

| | | Solu- | |
|---|---|---|---|
| | Buffers | bility | |
| pH | used | (mg/ml) | XRPD of excess solids (FIG. 41) |
| pH | KCl/HCl | 3.78 | Appears predominantly amorphous by XRPD |
| 1.0 | | | analysis. Solid material converted to a gum |
| | | | after being placed onto an XRPD sample |
| | | | holder as a slurry. |
| pH | Citric | 0.21 | The diffractogram appears different from |
| 3.0 | acid/ | | the input hydrobromide material, all known |
| | Sodium | | forms of the Compound 1 free base and the |
| | Citrate | | citric acid used in buffer preparation. The |
| | | | diffractogram also appears to correspond |
| | | | with the diffractograms obtained for the |
| | | | thermodynamic solubility experiments |
| | | | carried out on the bis-besylate salt. |
| pH | Citric | 0.08 | The diffractogram appears different from |
| 4.5 | acid/ | | the input hydrobromide material, all known |
| | Sodium | | forms of the Compound 1 free base and the |
| | Citrate | | citric acid used in buffer preparation. The |
| | | | diffractogram also appears to |
| | | | correspond with the diffractograms obtained |
| | | | for the thermodynamic solubility experiments |
| | | | carried out on the bis-besylate salt. |
| pH | Citric | 0.03 | The diffractogram appears different from the |
| 6.2 | acid/ | | input hydrobromide material, all known forms |
| | Sodium | | of the Compound 1 free base and the citric |
| | Citrate | | acid used in buffer preparation. The |
| | | | diffractogram also appears to correspond with |
| | | | the diffractograms obtained for the |
| | | | thermodynamic solubility experiments carried |
| | | | out on the bis-besylate salt. |

The diffractograms for the pH 3.0, 4.5 and 6.2 experiments appeared different from the input material as well as all identified forms of the hydrobromide salt and Compound 1 free base. The diffractograms also appeared different from the diffractograms of the solids used to make up the buffers. The solubility values obtained using these pH buffers are therefore likely not representative of the hydrobromide salt which was initially placed into the solutions.

Approximately 100-120 mg of material was compressed into discs by placing the material into a die (diameter: 13 mm) and compressing the die under 5 tons of pressure in a hydraulic press for ca. 2 minutes. A Sotax AT7 (conformed to EP2 and USP2) dissolution instrument was used containing paddles to stir the media at 100 rpm. Dissolution media of pH 3 (1% SDS) and pH 4.5 (1% SDS) were prepared using citrate/phosphate buffer. All materials were tested in 750 ml of the buffer medium. Discs were added at time=0 seconds and allowed to sink to the bottom of the dissolution vessel before stirring began. ca. 1 ml aliquots of media were extracted from the dissolution vessels at times 1, 5, 10, 15, 30, 60, 120, 240 minutes and 24 hours, and tested for API concentration by HPLC-UV. The dissolution tests were carried out in duplicate. For both dissolution media, the peak areas for the initial time points (up to 15 minutes), fell below the limit of quantification, however, when plotting Dissolution rate vs. time, the steepest part of the curve occurs during these early time points.

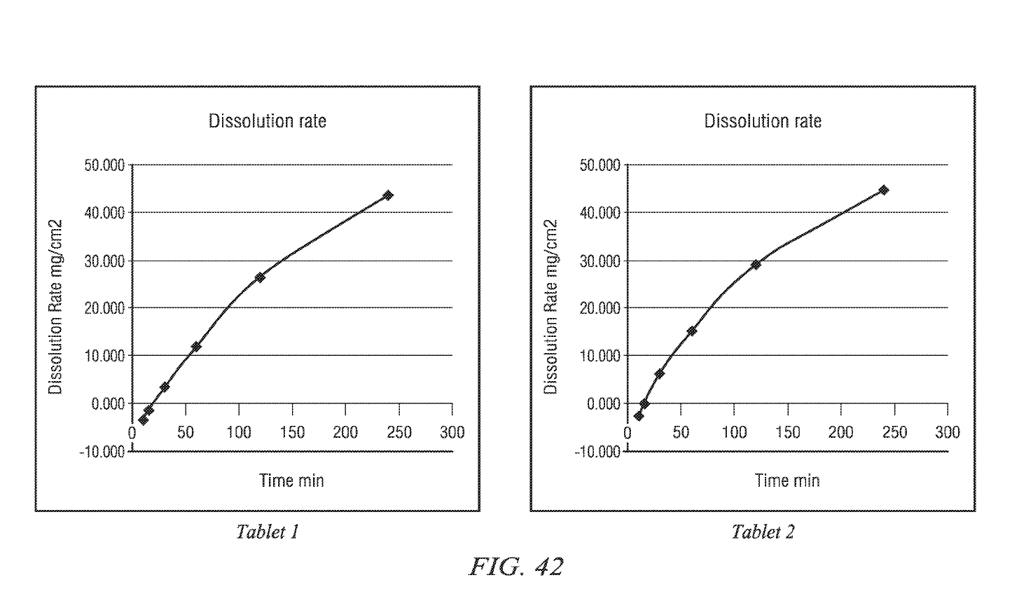

For pH 4.5, when plotting the curve of Dissolution rate vs. Time (FIG. 42), the intrinsic dissolution values obtained from the early time points on the curve (steepest part of the curve) were approximately 0.27 mg/cm²/min for tablet 1 and 0.28 mg/cm²/min for tablet 2.

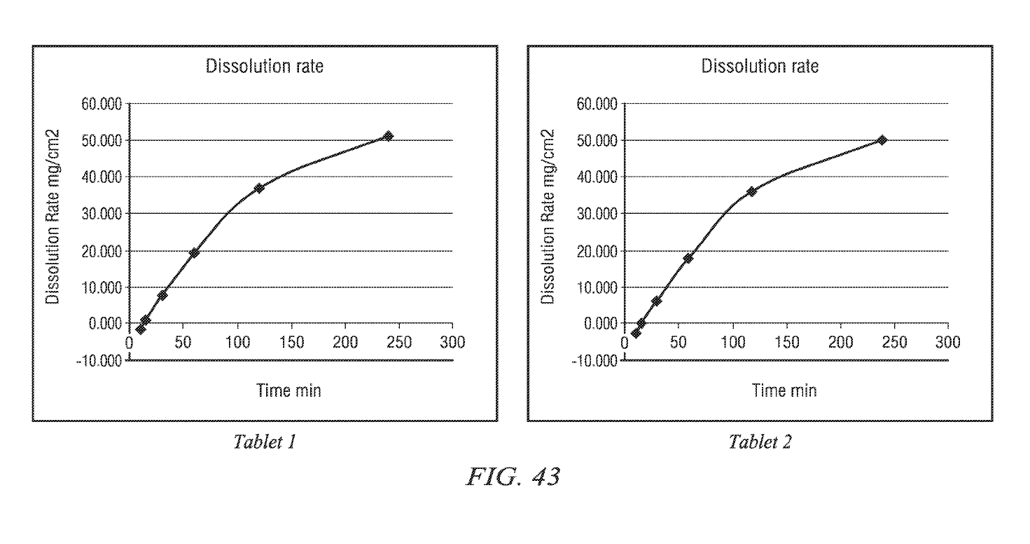

For pH 3.0, when plotting the curve of Dissolution rate vs. Time (FIG. 43), the intrinsic dissolution values obtained from the early time points on the curve (steepest part of the curve) were approximately 0.35 mg/cm²/min for both tablets 1 and 2.

### Example 8

### Secondary Screening of Hydrobromide Salt (2 equiv.)

Approximately 1 ml of methanol was added to ca. 200 mg of Compound 1 to form a slurry. In a separate vial, ca. 1 mL of methanol was added to 2 equivalents of hydrobromic acid (48%). The acid solution was then added dropwise over a 1 hour period to the free base slurry whilst stirring and maintaining a temperature between 0-5° C. After the complete addition of the acid, a further 1 mL of methanol was added. The reaction was stirred for ca. 3 hours before being isolated and dried. A yield of approximately 68% was obtained.

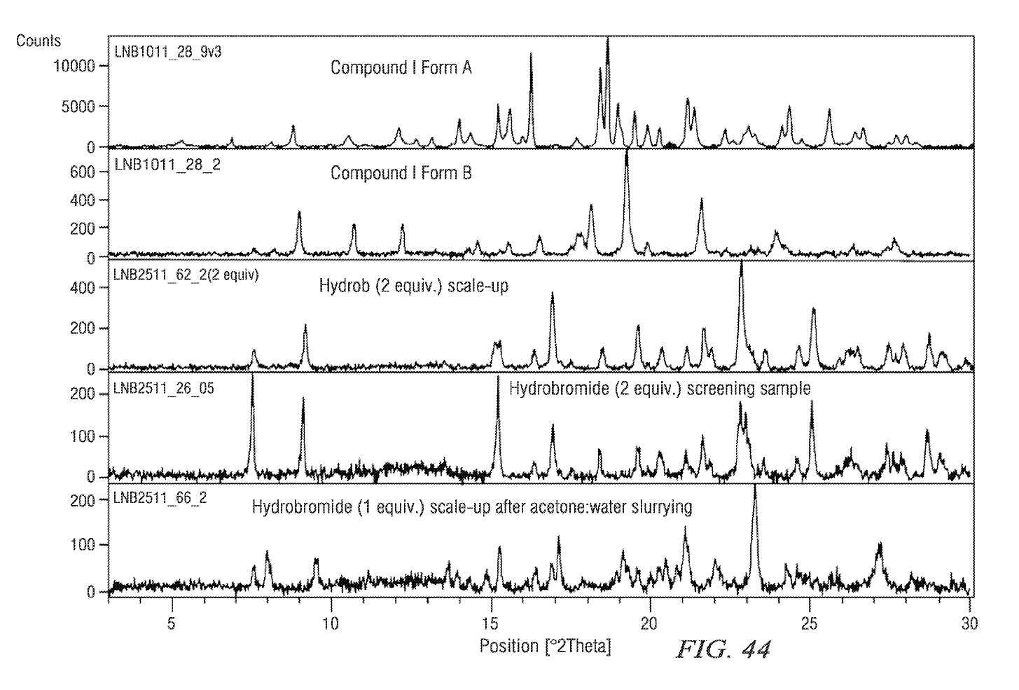

XRPD analysis (FIG. 44) was carried out after filtration and the diffractogram obtained was consistent with the Form I material obtained using both 1 and 2 equivalents of HBr in the primary salt screen.

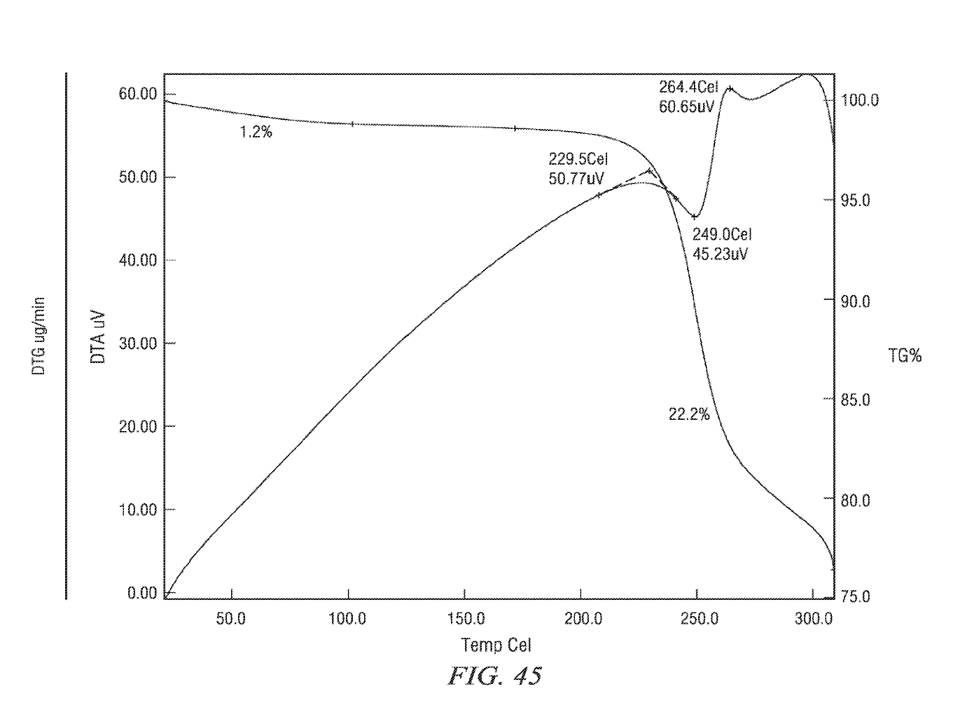

TGA/DTA (FIG. 45) showed a 1.2% weight loss from the outset to ca. 100° C., likely due to unbound moisture or solvent. No further weight losses were seen prior to degradation at onset ca. 230° C. The TGA/DTA is similar to the trace obtained for the 1 equivalent scaled-up form of Example 6.

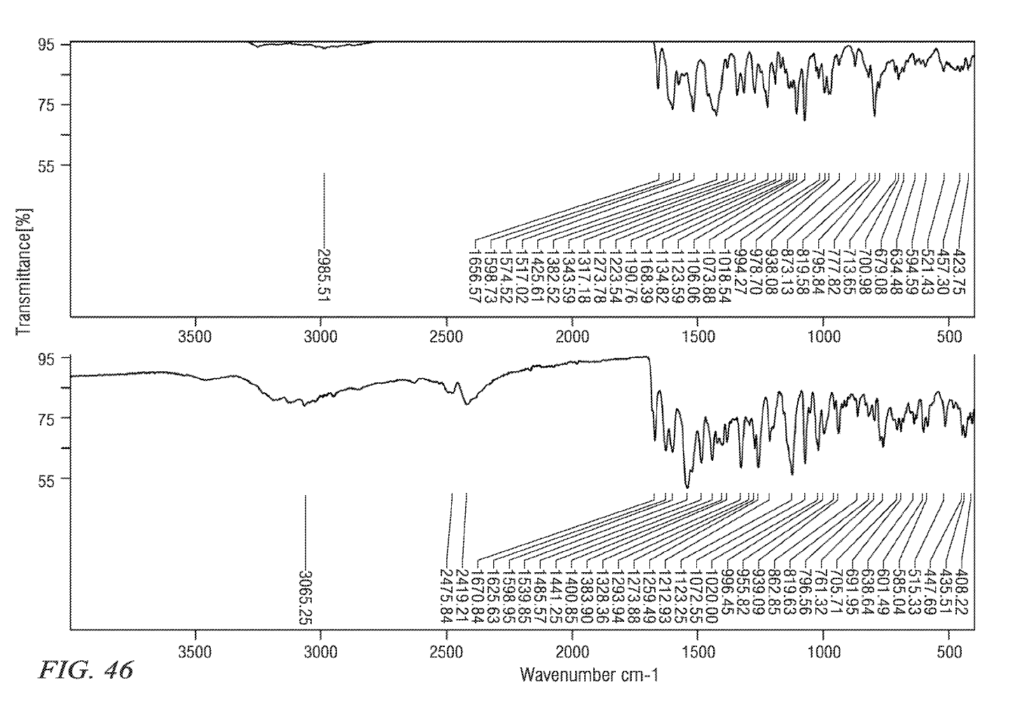

IR spectroscopy (FIG. 46) showed a number of differences and shifts in comparison with the free base and the hydrobromide (1 equiv.) scaled-up salt.

### Example 9

### Secondary Screening of Hydrobromide Salt (Unknown Form)

The thermodynamic solubility experiments carried out on the hydrobromide salt resulted in the formation of an unknown solid form. In attempts to characterize this form, as well as establish the rate of conversion to this form while slurrying the material, the following experiments were carried out. Initially, approximately 100 mg of the hydrobromide (1 equiv.) material was slurried in a pH 6.2 aqueous solution at ambient and XRPD analysis was carried out at time points 5 min., 1 hr, 2 hrs, 4 hrs and 8 hrs. Further analysis was then also carried out on the converted material.

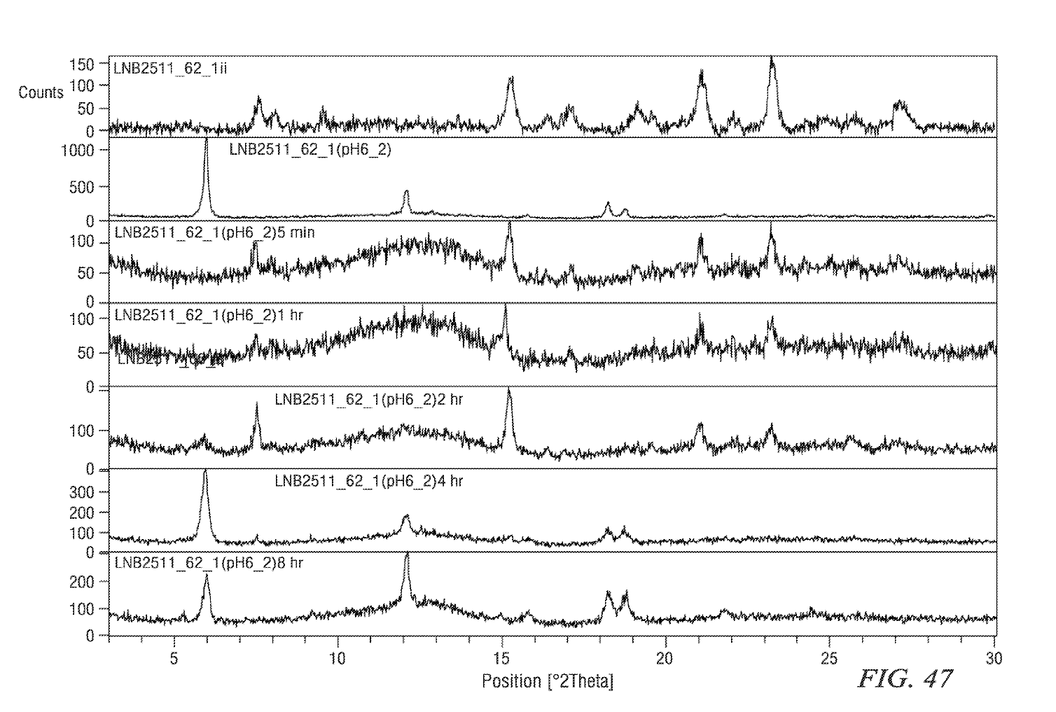

The XRPD analysis (FIG. 47) carried out on the hydrobromide (1 equiv.) salt sample after slurrying the solid in an aqueous pH 6.2 medium for 5 min, 1 hr, 2 hrs, 4 hrs and 8 hrs indicated that conversion to the unknown solid form occurs between 2-4 hours.

PLM analysis carried out on a slurry of the material indicated a very small particle size. Some birefringence was observed (not shown). Upon drying the material became glass-like.

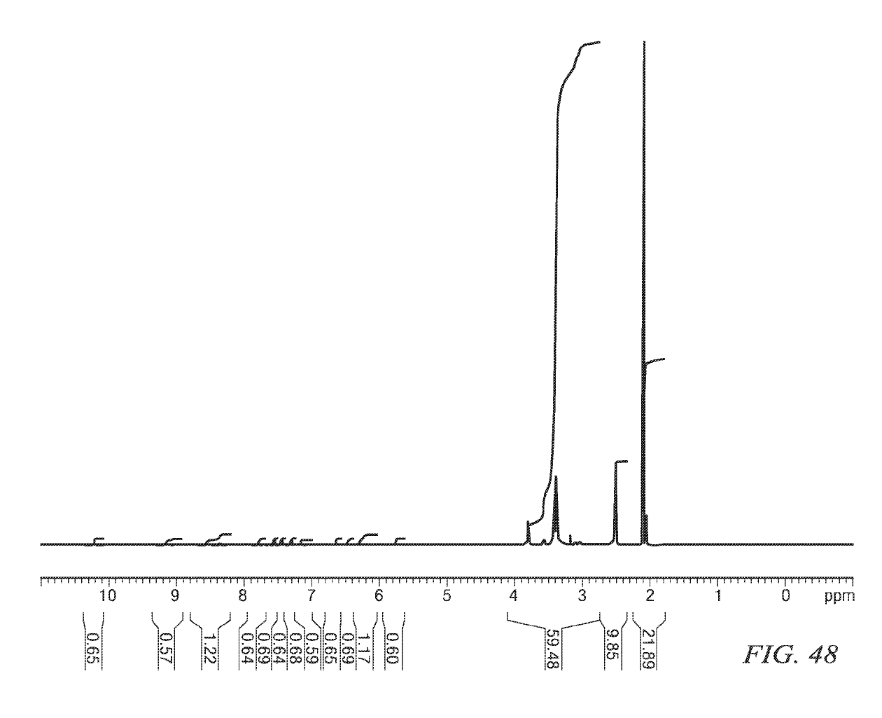

¹H NMR analysis was carried out on this material which showed a spectrum which was different in terms of peak positions from both the free base spectrum and the hydrobromide spectrum (FIG. 48).

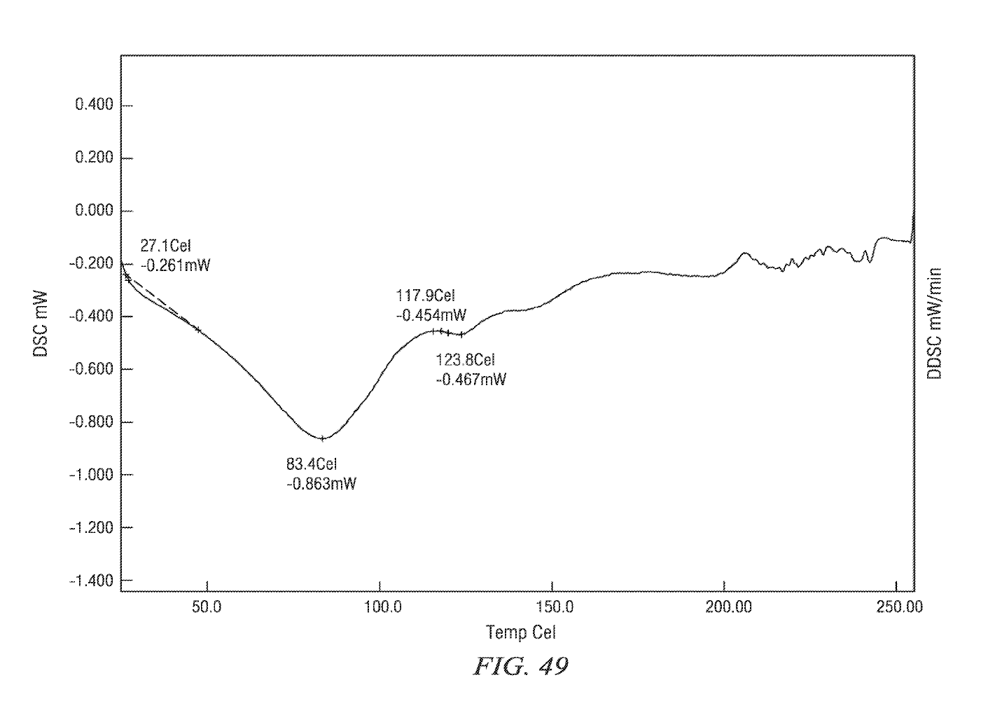

DSC analysis was also attempted on the glass-like material, however, a large broad endotherm is seen from the outset to ca. 110° C., followed by a pattern characteristic of amorphous material (FIG. 49).

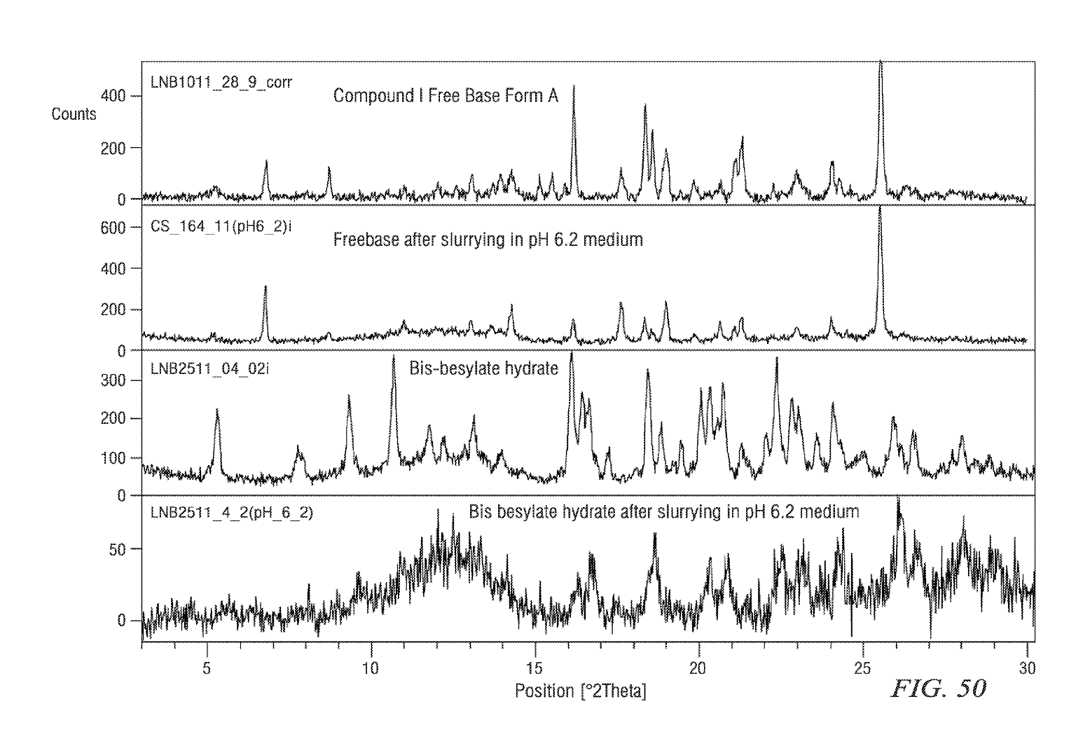

The slurry experiment of Compound 1 free base and the bis-besylate hydrate (carried out in attempts to produce more of this form for analysis) were unsuccessful in producing this unknown solid form. For the free base slurry, the material remained as the free base Form I and for the bis-besylate hydrate slurry, the material lost some crystallinity, but remained as the bis-besylate hydrate (FIG. 50).

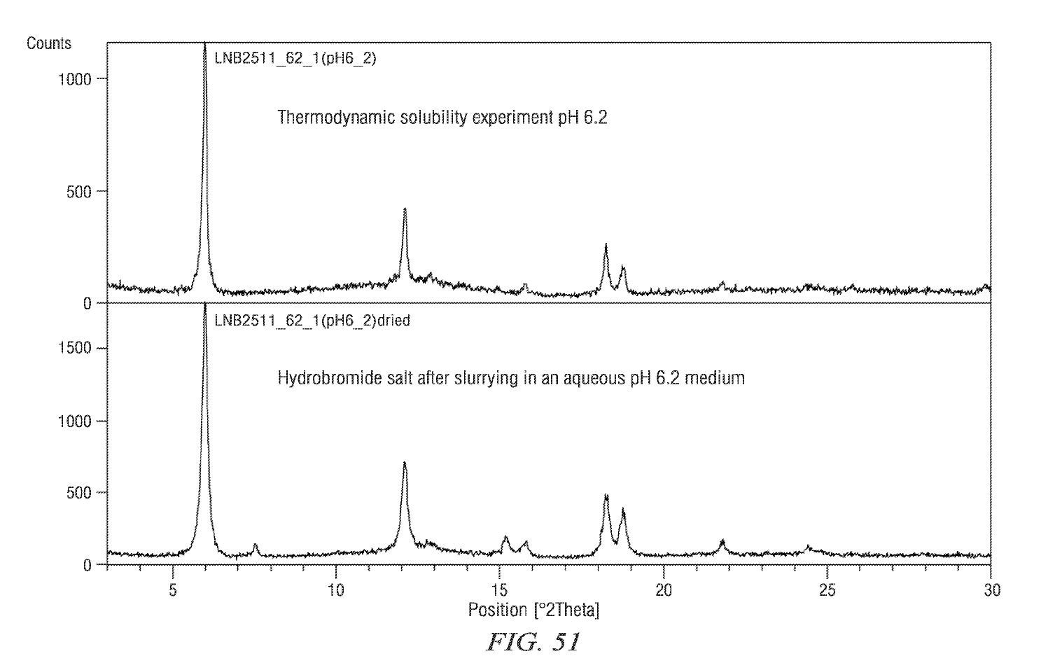

The further scale-up of the hydrobromide (1 equiv.) salt and subsequent slurrying in a pH 6.2 aqueous medium resulted in this unknown solid form being obtained (FIG. 51), however, all attempts at filtering the material were unsuccessful with the solid going through a sintered filter and multiple sheets of filter paper, due to the small particle size. Again, attempts at evaporating off the solvent resulted in a glass-like material being obtained. This appears to indicate that the unknown form is unstable when isolated.

### Example 10

### Tertiary Screening of Hydrobromide Salt (1 equiv.)

Approximately 85 mL of acetonitrile:water (90:10) was added to ca. 20 g of Compound 1 in a round bottomed flask to form a slurry. In a separate flask, 1 equivalent of hydrogen bromide (ca. 4.073 mL) was added to ca. 70 mL of acetonitrile:water (90:10). The acid solution was then added in small aliquots to the free base slurry while stirring at ca. 4° C. The resulting slurry was then allowed to stir at ambient temperature for ca. 2 hours. It was then placed at ca. 5° C. for 1 day before stirring for a further 4 hours at ambient temperature. The reaction was then filtered and the solid dried under vacuum at ambient temperature (ca. 22° C.). The drying was continued for ca. 2 days. Due to the partially crystalline nature of the material after drying, the material was then slurried in ca. 50 mL of an acetone:water (90:10) mixture. The reaction was temperature cycled between ca. 4-22° C. in 1 hour cycles while stirring for ca. 2 days. The reaction was then filtered and dried at ambient for ca. 4 days before being analyzed. The yield after the further slurrying was 16.4 g (63%).

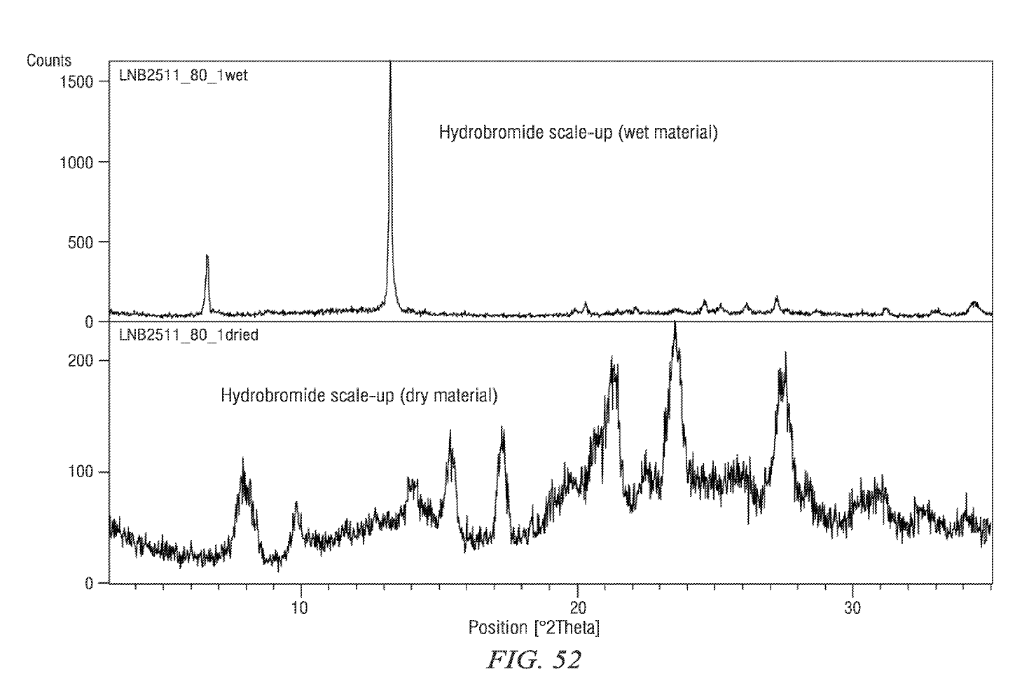

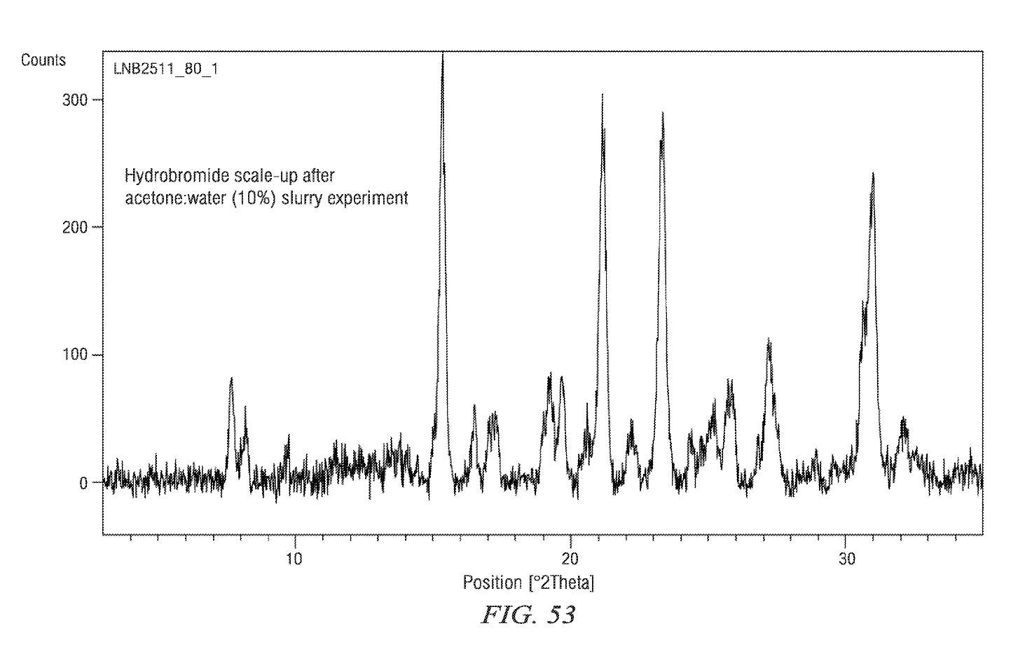

XRPD analysis (FIG. 52) carried out on the initial scale-up material while wet, showed the sample to be highly crystalline. After drying, the solid converted to a different polymorphic form and also lost some crystallinity. XRPD analysis (FIG. 53) on the material after further slurrying in acetone:water(10%) and subsequent drying indicated a crystalline material. The diffractogram corresponded with the smaller scale hydrobromide sample obtained after drying during Example 1.

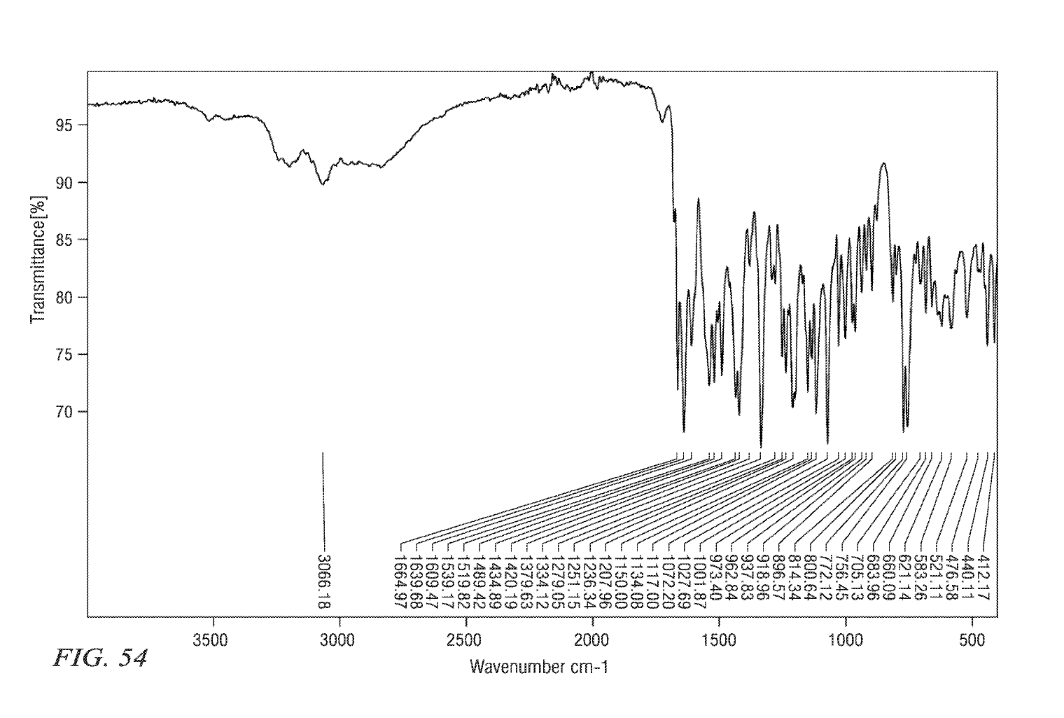

IR Spectroscopy (FIG. 54) showed differences when compared with the free base spectrum. The spectrum also appeared consistent with the spectrum obtained for the hydrobromide salt in Example 1.

PLM (not shown) showed small particles with no defined morphology and little birefringence.

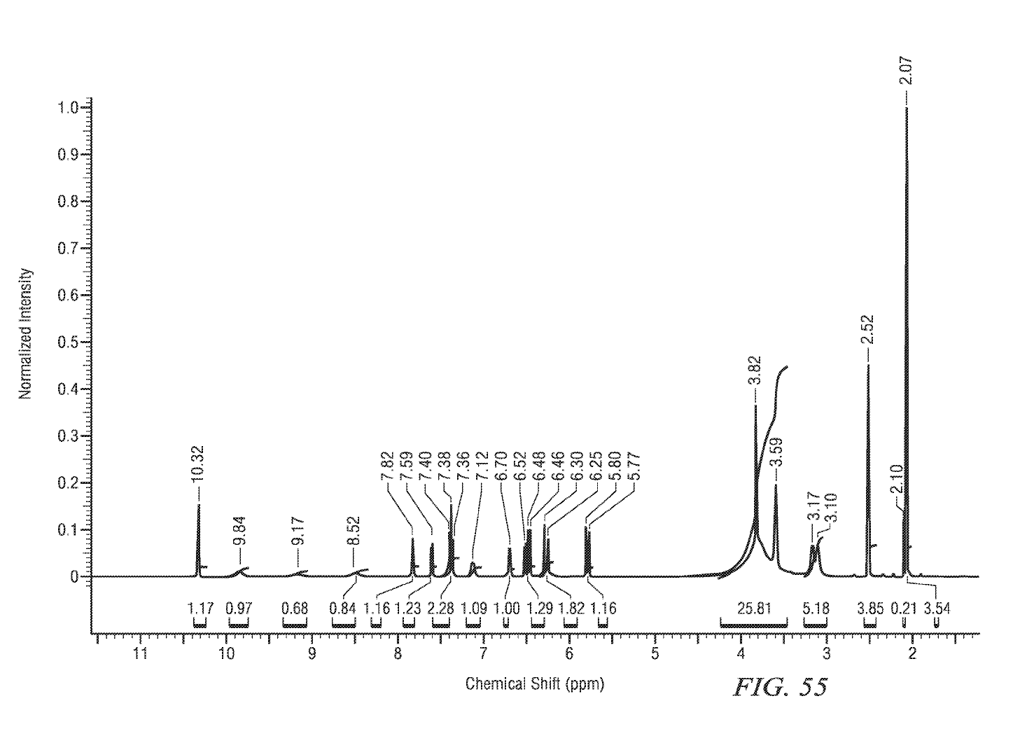

¹H NMR (FIG. 55) indicated a number of peak shifts in comparison with the free base. A small non-stoichiometric amount of acetone was present in the spectrum.

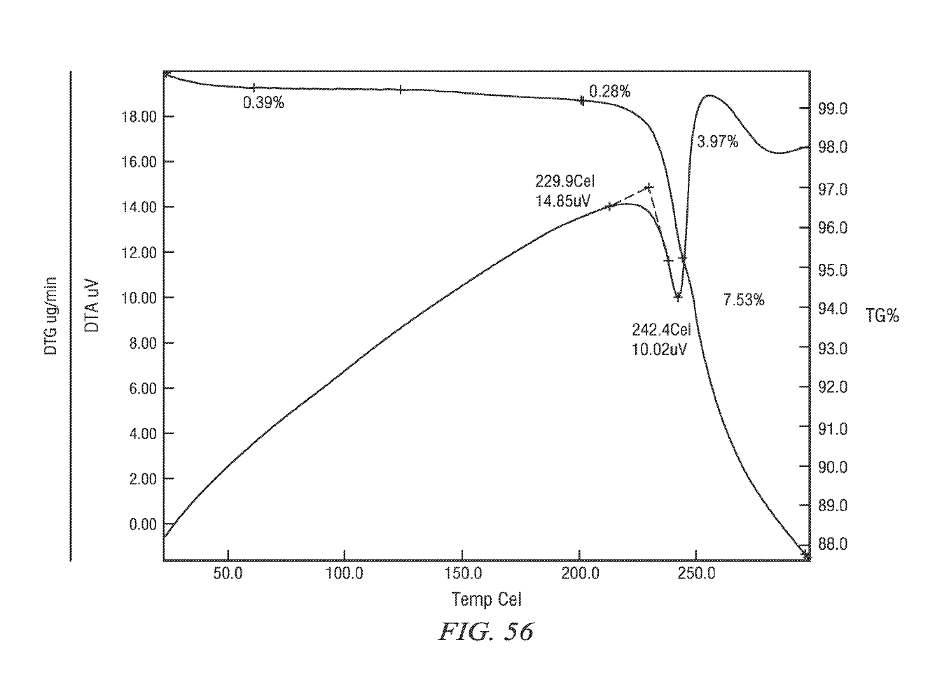

TGA/DTA (FIG. 56) showed a weight loss from the outset of ca. 0.4%, likely due to unbound moisture or solvent. No further significant weight losses were seen prior to degradation at onset ca. 230° C.

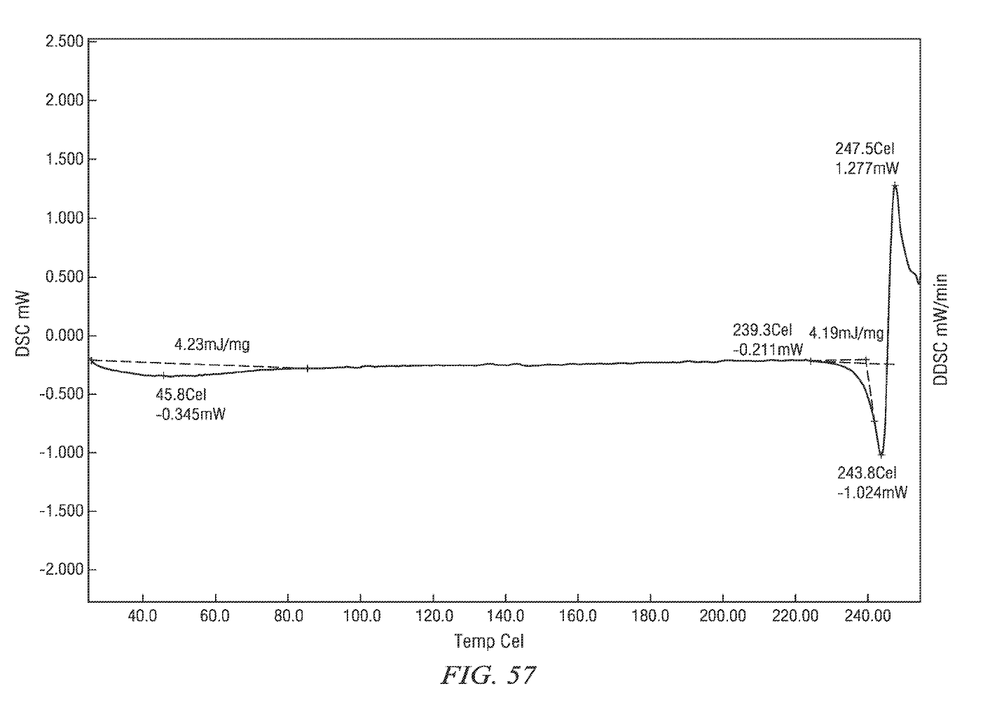

DSC analysis (FIG. 57) indicated a shallow, broad endotherm from the outset likely due to unbound solvent/water. A second endotherm was then present at onset ca. 240° C. (peak 244° C.), followed by likely degradation.

KF analysis determined the water content of the material to be ca. 0.76%.

HPLC purity determination indicated a purity of ca. 98.1%.

The content of carbon, hydrogen and nitrogen in the material was determined by placing the samples into a tin capsule, placed inside an autosampler drum of an elemental analysis system. The sample environment was purged by a continuous flow of helium and the samples dropped at pre-set intervals into a vertical quartz tube maintained at 900° C. The mixture of combustion gases was separated and detected by a thermal conductivity detector giving a signal proportional to the concentration of the individual components of the mixture. The content of bromine in the material was determined by oxygen flask combustion of the sample. Once the combustion and absorption into solution had occurred, the samples were titrated using a calibrated Mercuric Nitrate solution. Elemental analysis (CHN and bromide) indicated the following percentages:

| ELEMENT | C | H | N | Br |
|---|---|---|---|---|
| % Theory | 51.03 | 4.44 | 15.42 | 12.57 |
| % Found | 50.36 | 4.32 | 16.47 | 12.11 |

Ion chromatography was carried out using a Metrohm 761 Compact Ion Chromatograph for the analysis of ions in aqueous solutions. Calibration standards were prepared from certified 1000 ppm stock solutions. Ion chromatography showed the presence of 12.38% bromide.

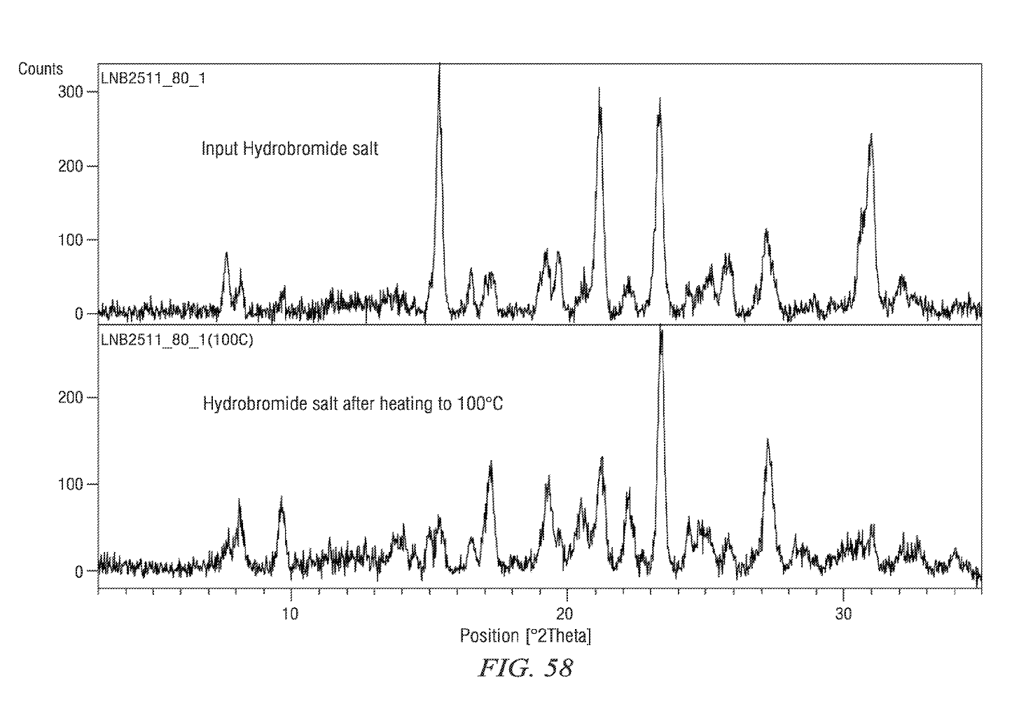

In order to examine the effect of removing the water which is retained by the material, (despite extended periods of drying) a small sample was heated to 100° C. in a TGA pan and XRPD analysis was then carried out (FIG. 58). The analysis indicates some loss in crystallinity, however, the polymorphic form remains consistent after removing the ca. 0.5% water by heating. Nevertheless, the material appears to be slightly hygroscopic.

### Example 11

### Large-Scale Preparation of Hydrobromide Salt (1 equiv.)

Approximately 1 L of acetone:water (90:10) was added to ca. 319 g of Compound 1 in a 5 L reaction vessel with the reactor temperature set to 4° C. A suspension was obtained. The suspension was stirred at 450 rpm. In a separate flask, 1 equivalent of hydrogen bromide (48%)(ca. 65 mL) was added to ca. 750 mL of acetone:water (90:10). The acid solution was then added to the 5 L reactor over a 1 hour period, while maintaining a temperature of ca. 4° C. After 30 minutes, a further 700 mL of acetone:water (90:10) was added to the reactor. After the complete addition of the HBr solution, the reactor temperature was raised to 20° C. for 2 hours. The reaction was then again cooled to ca. 4° C. and maintained at this temperature for a further 3 hours. The reaction mixture was then filtered and dried under vacuum at ambient temperature (ca. 22° C.) for 3 days. The solid was stirred periodically during the drying process. The yield after drying was 258.1 g (71%).

XRPD analysis (FIG. 59) carried out on the initial scale-up material while wet, showed the sample to be highly crystalline. After drying, the solid converted to a different polymorphic form (FIG. 60). The dried material is the same form as that obtained from the primary salt screen.

IR Spectroscopy (FIG. 61) showed differences when compared with the freebase spectrum. The spectrum also appeared consistent with the spectra obtained for the hydrobromide salt prepared in Examples 1 and 7.

PLM analysis showed a needle-like, fibrous morphology when wet (not shown).

Upon drying and hence polymorph conversion, the needle-like morphology was lost with small particles resulting.

¹H NMR (FIG. 62) indicated a number of peak shifts in comparison with the free base. Trace amounts of acetone were present in the spectrum.

TGA/DTA (FIG. 63) showed a weight loss from the outset of ca. 0.4%, likely due to unbound moisture or solvent. No further significant weight losses were seen prior to degradation at onset ca. 230° C. Thus, the material appears to retain ca. 0.5% water at ambient conditions despite extended periods of drying and therefore appears to be slightly hygroscopic.

DSC analysis (FIG. 64) indicated a shallow, broad endotherm from the outset likely due to unbound solvent/water. A second endotherm was then present at onset ca. 241° C. (peak 245° C.), followed by likely degradation.

KF analysis determined the water content of the material to be ca. 0.74%.

HPLC purity determination indicated a purity of ca. 99.1%.

Slurries of the hydrobromide salt were prepared in buffered aqueous media at pH 1.0 (HCl/KCl buffer), pH 3.0 (citrate buffer), pH 4.5 (citrate buffer) and pH 6.2 (citrate buffer) as well as in an aqueous solution with pH reduced to below 2 using HBr (48%). The respective slurries were shaken for a period of 24 hours at 22° C. The solids were then removed by filtration and tested by XRPD analysis. The mother liquors were analyzed by HPLC to determine API solubility. HPLC solubility determination in various pH media showed the following results:

| pH condition | Conc. (mg/mL) |
|---|---|
| Aqueous solution with pH | 3.52 |
| reduced to below 2 using HBr. | |
| pH 1.0 (HCl/KCl buffer) | 4.09 |
| pH 3.0 (citrate buffer) | 0.20 |
| pH 4.5 (citrate buffer) | 0.17 |
| pH 6.2 (citrate buffer) | 0.04 |

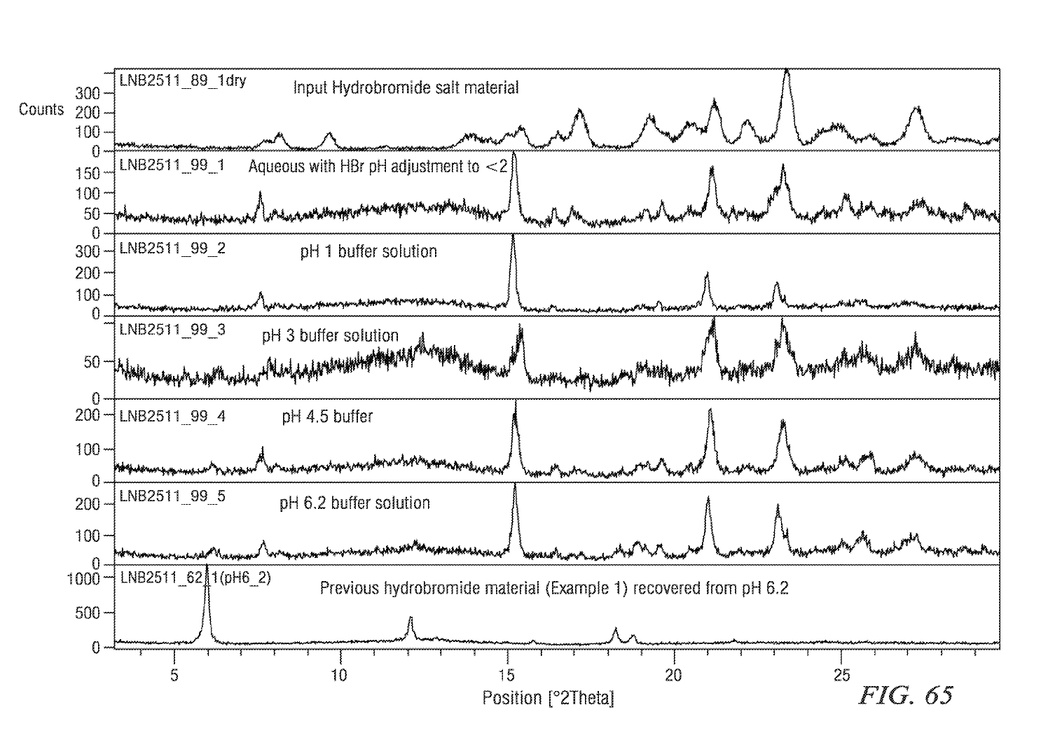

XRPD analysis of the solids recovered after the solubility experiments (FIG. 65) showed all samples to correspond predominantly with the input hydrobromide salt material, with the samples in the pH 3.0, 4.5 and 6.2 buffers showing traces of a form previously identified from disproportionation studies and previous slurring of the hydrobromide salt in pH buffers >pH 3.

Elemental analysis (CHN and bromide) indicated the following percentages:

| ELEMENT | C | H | N | Br |
|---|---|---|---|---|
| % Theory | 51.03 | 4.44 | 15.43 | 12.57 |
| % Found | 50.42 | 4.60 | 15.14 | 12.54 |

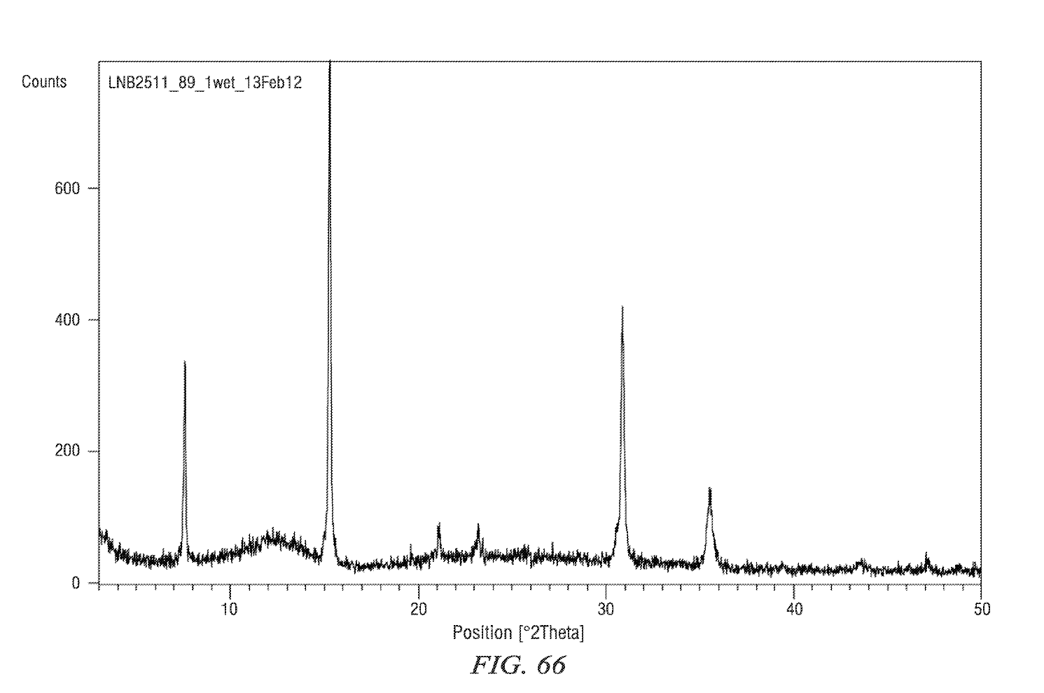

A small slurry of the scale-up material was stored for ca. 1.5 months at ca. 4° C. Upon re-analyzing the material by PLM analysis, the crystals appeared as very flat, rod-shaped particles in comparison to the fibrous, needle-like particles previously observed (not shown). The material converted to the same form as the one obtained upon drying with a change in crystal morphology from fibrous needle-like crystals to flat, rod-like crystals. XRPD analysis (FIG. 66) indicated a diffractogram which corresponded with the dry hydrobromide salt material (peaks at 7.59, 15.28, 21.10, 23.21, 30.88, 35.54, 43.58 and 47.13° 2-theta). The peaks appear very sharp with some preferred orientation in the diffractogram.

### Example 12

### Primary Polymorph Screening of Hydrobromide Salt

Preparation of Amorphous Material.

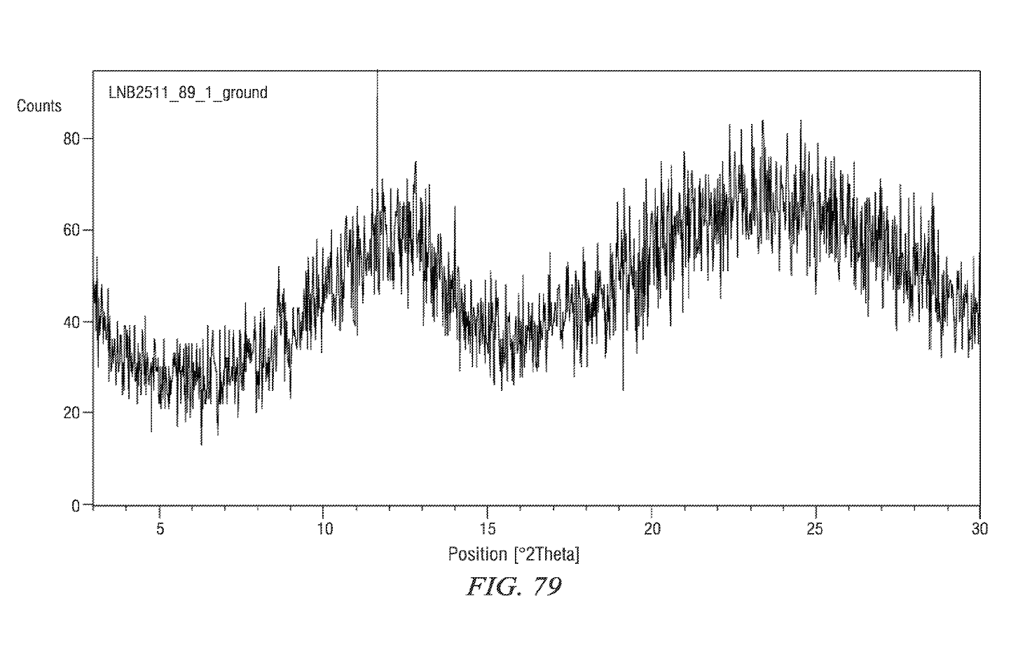

Hydrobromide salt material was ground using a Retsch Ball Mill for ca. 25 minutes, with a 5 minute break midway to prevent the sample from overheating. The sample was then analysed by XRPD to determine form and by HPLC to check for degradation. Post grinding XRPD analysis showed the hydrobromide salt material to be amorphous with an HPLC purity of ca.99.5%. (FIG. 79). Amorphous material was desired in order to both increase the solubility and not to bias the screening study towards one particular form.

Solvent Solubility Screen.

Approximately 10 mg of amorphous hydrobromide salt was placed in each of 24 vials and 5 volume aliquots of the appropriate solvent system was added to the vial. Between each addition, the mixture was checked for dissolution. This procedure was continued until dissolution was observed or until 100 volumes of solvent had been added. Amorphous hydrobromide salt material was found to be highly soluble in 3 of the 24 solvent systems but exhibited low solubility in the remaining solvents. The approximate solubility values of the amorphous hydrobromide salt in the 24 solvent systems are presented in Table 12:

TABLE 12
Approximate Solubility in Selected Solvents

| | Approximate |
|---|---|
| | Solubility |
| Solvent | (mg/mL) |
| Acetone | <10 |
| Acetone:Water (10%) | <10 |
| Acetonitrile | <10 |
| 1-Butanol | <10 |
| Cyclohexane | <10 |
| Dichloromethane | <10 |
| Diisopropyl ether | <10 |
| Dimethylformamide | ca. 67 |
| Dimethylsulfoxide | ca. 50 |
| 1,4-Dioxane | <10 |
| Ethanol | <10 |
| Ethyl acetate | <10 |
| Heptane | <10 |
| Isopropyl acetate | <10 |
| 3-Methyl-1-butanol | <10 |
| Methylethyl ketone | <10 |
| Methyl isobutyl ketone | <10 |
| N-Methyl-2-pyrrolidone | ca. 20 |
| Nitromethane | <10 |
| 2-Propanol | <10 |
| tert-Butylmethyl ether | <10 |
| Tetrahydrofuran | <10 |
| Toluene | <10 |
| Water | <10 |

Temperature Cycling Experiments.

The results obtained from the solubility approximation experiments were used to prepare slurries for temperature cycling. The slurries were temperature cycled between 4° C. and 25° C. in 4 hour cycles for a period of 72 hours (slurries were held at 4° C. for 4 hours followed by a hold at ambient for 4 hours, the cooling/heating rates after the 4 hour hold periods was ca. 1° C./min). Solid material was then recovered for analysis.

Crash Cooling Experiments.

Crash cooling experiments were performed by placing saturated solutions of the material, in each of the 24 selected solvent systems, in environments of 2° C. and −18° C. for a minimum of 48 hours. Any solid material was then recovered for analysis.

Rapid Evaporation Experiments.

Rapid evaporation experiments were conducted by evaporating the solvents from saturated, filtered solutions of the material, in each of the 24 solvent systems, under vacuum. Any solid material was then recovered and analysed after the solvent had evaporated to dryness.

Anti-Solvent Addition Experiments.

Anti-solvent addition experiments were conducted at ambient temperature by adding the selected anti-solvent to saturated, filtered solutions of the material, in each of the 24 selected solvent systems. The anti-solvent selected was heptane, with tert-butylmethyl ether and water being used for solvents immiscible with heptane. Addition of anti-solvent was continued until there was no further precipitation or until no more anti-solvent could be added. Any solid material was recovered and analysed quickly in order to prevent form changes.

Slow Evaporation Experiments.

Slow evaporation experiments were conducted by evaporating the solvents from saturated, filtered solutions of the material, in each of the 24 solvent systems at ambient conditions. Any solid material was then recovered and analysed after the solvent had evaporated to dryness.

Desolvation of Solvated Forms.

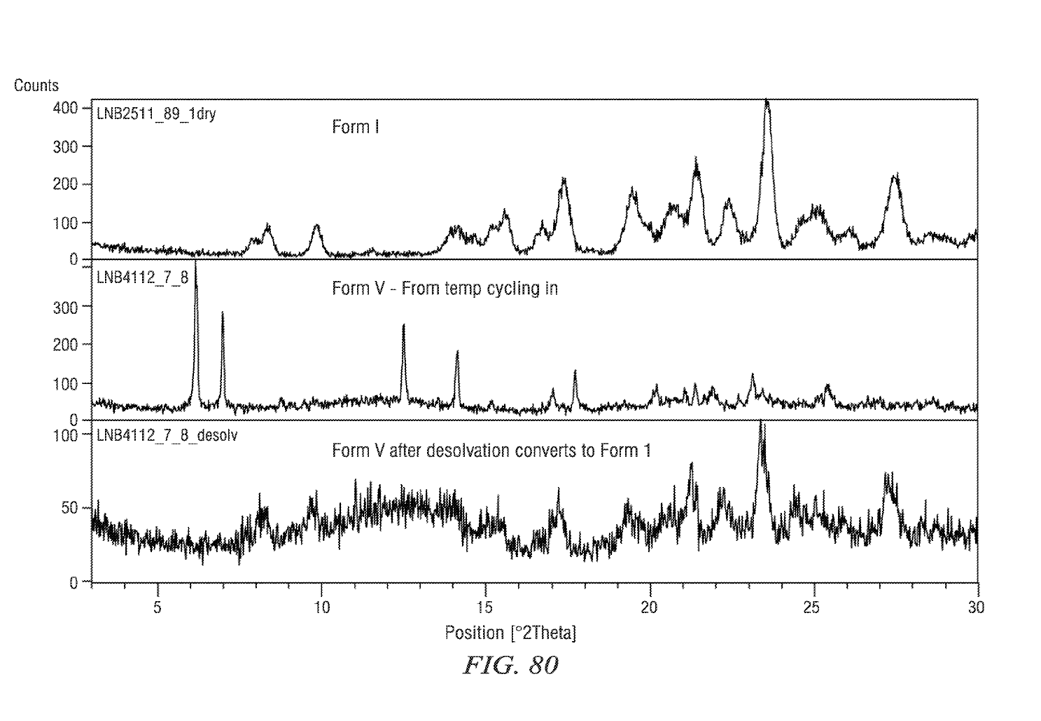

Potential solvated forms were subjected to heating on a TGA instrument to a temperature slightly beyond the initial weight loss. It could then be determined by subsequent XRPD analysis whether the form had changed as a result of the loss of solvent molecules. After heating to 180° C. using TGA instrumentation, Form V solvate was found to have reverted to Form I by XRPD analysis. The resultant diffractogram is shown in FIG. 80. The attempted desolvation of Form VII resulted in a gum following heating.

Investigation into Wet and Dry Samples of Form I.

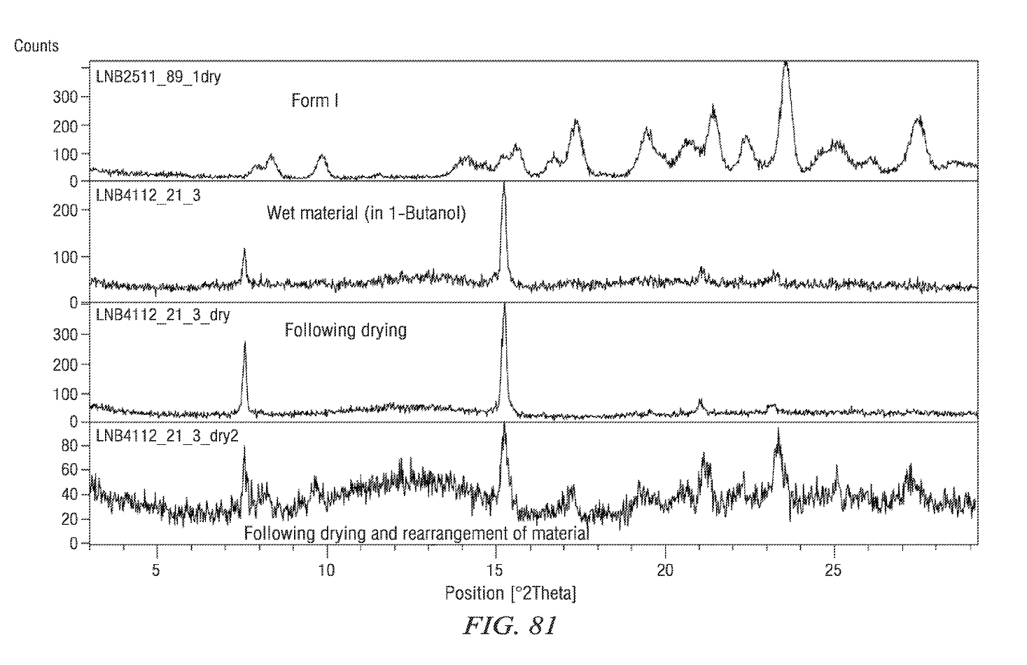

Initially, wet samples of Form 1 showed some differences in the XRPD diffractograms to those of the dry samples. Further investigation, including drying studies followed by XRPD analysis, TGA, and XRPD analysis with spinning were carried out. For Form I, the wet material showed significant preferred orientation and shifting was observed in the diffractograms when compared to the dry material. FIG. 81 shows input material Form I compared with a wet sample, and after stages of drying.

The results from the experiments conducted during the primary polymorph screen are shown in Table 13. Results were obtained from PLM and XRPD analysis. Overall it can be seen that multiple potential polymorphic forms were identified during the screening experiments.
Form I was obtained from multiple temperature cycling experiments.
Form III, an anhydrous form, was obtained from rapid evaporation of DMSO, crash cooling to 2° C. in ethanol, and anti-solvent addition from acetone, acetonitrile, and ethanol.
Form IV, a 1,4-dioxane solvate, was obtained from temperature cycling in 1,4-dioxane.
Form V, a DMF solvate, was obtained from temperature cycling and rapid evaporation from DMF.
Form VI, a DMSO solvate, was obtained from temperature cycling in DMSO.
Form VII, a DMSO solvate, was obtained from slow evaporation from DMSO.

TABLE 13
Results of Primary Hydrobromide Salt Screen

| | | Temper- | | Rapid | Crash | Crash | Anti- |
|---|---|---|---|---|---|---|---|
| | | ature | Evapora- | Evapora- | Cool | Cool | Solvent |
| | Solvent | Cycling | tion | tion | (2° C.) | (−18° C.) | Addition |
| 1 | Acetone | Form I | AM/ | AM/ | NS | NS | Form III |
| | | | PLM⁺ | PLM⁺ | | | (heptane) |
| 2 | Acetone:Water | FB | Form I | FB^{§} | FB^{§} | FB^{§} | NS |
| | (10%) | | | | | | (heptane) |
| 3 | Acetonitrile | Form I* | AM/ | AM/ | NS | NS | Form III |
| | | | PLM⁺ | PLM⁺ | | | (tBME) |
| 4 | 1-Butanol | Form I | NS | AM/ | NS | NS | AM/ |
| | | | | PLM⁺ | | | PLM |
| | | | | | | | (heptane) |
| 5 | Cyclohexane | AM | NS | NS | NS | NS | NS |
| | | | | | | | (heptane) |
| 6 | Dichloromethane | Form I | AM/ | AM | NS | PLM | AM/ |
| | | | PLM⁺ | | | | PLM |
| | | | | | | | (heptane) |
| 7 | Diisopropyl ether | Form I | NS | NS | NS | NS | NS |
| | | | | | | | (heptane) |
| 8 | Dimethylformamide | Form V | Form I | Form V | NS | NS | WD |
| | | | | | | | (tBME) |
| 9 | Dimethylsulfoxide | Form VI | Form | Form III | NS | AM/ | AM |
| | | | VII | (WD) | | PLM^{#†} | (water) |
| 10 | 1,4-Dioxane | Form IV | NS | AM/ | NS | NS | NS |
| | | | | PLM⁺ | | | (heptane) |
| 11 | Ethanol | Form I | AM/ | AM | Form | PLM | Form III |
| | | | PLM^{‡} | | III\hat{ } | | (heptane) |
| 12 | Ethyl acetate | Form I | NS | NS | NS | NS | AM/ |
| | | | | | | | PLM |
| | | | | | | | (heptane) |
| 13 | Heptane | AM | NS | NS | NS | NS | NS |
| | | | | | | | (tBME) |
| 14 | Isopropyl acetate | Form I | NS | NS | NS | NS | AM/ |
| | | | | | | | PLM |
| | | | | | | | (heptane) |
| 15 | 3-Methyl-1-butanol | Form I | AM/ | AM/ | NS | NS | AM/ |
| | | | PLM⁺ | PLM⁺ | | | PLM |
| | | | | | | | (heptane) |
| 16 | Methylethyl ketone | Form I | AM/ | AM | NS | NS | AM |
| | | | PLM⁺ | | | | (heptane) |
| 17 | Methyl isobutyl | Form I | NS | NS | NS | NS | AM/ |
| | ketone | | | | | | PLM |
| | | | | | | | (heptane) |
| 18 | N-Methyl-2- | Form I | NS | NS | NS | NS | AM |
| | pyrrolidone | | | | | | (tBME) |
| 19 | Nitromethane | Form I | A M/ | AM/ | NS | PLM | AM |
| | | | PLM | PLM | | | (tBME) |
| 20 | 2-Propanol | Form I | AM | AM | NS | NS | AM/ |
| | | | | | | | PLM |
| | | | | | | | (heptane) |
| 21 | tert-Butylmethyl | Form I | NS | NS | NS | NS | NS |
| | ether | | | | | | (heptane) |
| 22 | Tetrahydrofuran | Form I | AM/ | AM/ | NS | NS | AM |
| | | | PLM^{#} | PLM^{#} | | | (heptane) |
| 23 | Toluene | Form I | NS | NS | NS | NS | NS |
| | | | | | | | (heptane) |
| 24 | Water | FB | NS | NS | NS | NS | NS |

AM—amorphous solid
NS—no solid observed
AM/PLM—amorphous by XRPD, birefringence observed by PLM
FB—Compound 1 free base
PLM—birefringence by PLM
WD—weak data
*poorly crystalline
⁺no clear morphology
\hat{ }only 2 peaks present; needle-like morphology
^{#}plate-like morphology
^{†}similar to Form VI
^{‡}rod-like morphology
^{§}missing peaks

### Example 13

### Secondary Polymorph Screening of Hydrobromide Salt and Developability Assessment

Form III hydrobromide salt of Compound 1 (1 equiv.) was obtained during the primary polymorph screen from multiple experiments. This form was therefore progressed for scale-up and further analysis.

Form III Hydrobromide Salt Preparation.

Approximately 500 mg of amorphous Compound 2 HBr salt material was slurried in ca. 6 mL of acetonitrile. The suspension was then temperature cycled between 4 and 25° C. in four hour cycles for ca. 2 days. The secondary screen analysis was carried out on the material when it was damp, due to the instability of Form III.

During the scale-up of the Form III hydrobromide salt, the material remained yellow in colour. XRPD analysis showed the material produced from scale-up to be crystalline and consistent with the small scale Form III hydrobromide salt diffractogram. PLM analysis indicated birefringent, needle-like crystals when wet. Hot stage microscopy indicated that as the solvent dried off between 40 and 50° C., the crystal morphology changed to more rod-like crystals. By ca. 250° C., the material was observed to melt. For TGA/DTA analysis, a damp sample of Form III was placed into the TGA pan. An initial 10.3% weight loss was observed due to the unbound solvent. The form change which occurs between 40 and 50° C. by hotstage microscopy was masked by the solvent loss. A further endotherm corresponding with Form I hydrobromide salt was observed at onset ca. 239° C. (peak ca. 245° C.). DSC analysis indicated an initial endotherm from the outset up to approximately 100° C. A final endotherm was observed at onset ca. 233° C. (peak ca. 247° C.), which appears consistent with the Form I melt. IR spectroscopy indicated very small differences between the IR spectrums of Forms I and III.

DVS analysis showed the following observations:
Cycle 1—Sorption 20-90% RH
   Sample gradually takes up ca. 1.045% mass.
Cycle 2—Desorption 90-0% RH
   Between 90-0% RH, sample mass decreases gradually by ca. 1.983%.
Cycle 3—Sorption 0-20% RH
   Moisture uptake of ca. 0.535% between 0-20% RH.

The material was observed to be slightly hygroscopic. Post DVS XRPD indicated that the material converted to Form I hydrobromide salt during DVS analysis. ¹H NMR spectroscopy carried out in deuterated DMSO showed a spectrum which corresponded with the Form I hydrobromide salt. KF analysis indicated the presence of 1.4% water. HPLC purity analysis indicated a purity of ca. 99.43%. Ion chromatography indicated the presence of 12.17% bromide (ca. 12.57% required for 1 equivalent).

XRPD analysis carried out on the thermodynamic solubility experiment solids remaining after 24 hours, indicated that for the pH 6.6 and 4.5 experiments, the Form III hydrobromide salt converted to a freebase hydrate form, the solid from the pH 3.0 experiment became amorphous and the solid from the pH 1 experiment remained predominantly consistent with the Form III hydrobromide, with some loss in crystallinity.

7 day Stability Studies at 25° C., 80° C., 40° C./75% RH (open and closed conditions).

Approximately 15 mg of Form III was placed into vials and then exposed to 25° C., 80° C. and 40° C./75% RH environments (open and closed vials) for 1 week to determine stability. The resulting solids were analysed by XRPD and HPLC to establish if any changes had occurred. The 1 week stability studies carried out in open and closed vials at 25° C., 80° C. and 40° C./75% RH indicated the following results:

TABLE 14
7 day stability studies (open container)

| Condition | Purity | XRPD analysis |
|---|---|---|
| 40° C./75% RH | 98.3% | Form I |
| 80° C. | 98.7% | Form I (some loss |
| | | in crystallinity) |
| 25° C. | 98.2% | Form I |

TABLE 15
7 day stability studies (closed container)

| Condition | Purity | XRPD analysis |
|---|---|---|
| 40° C./75% RH | 99.0% | Form I |
| 80° C. | 99.0% | Form I |
| 25° C. | 98.9% | Form I |

From the characterisation carried out on Form III, this form was determined to be a metastable, likely anhydrous form of the hydrobromide salt. Form III was observed to be very unstable with conversion to Form I occurring upon isolation and drying of the material.

Thermodynamic Solubility Studies.

Slurries of Form III were created in media of various pH (pH 1; pH 3; pH 4.5 and pH 6.6) and shaken for ca. 24 hours. After 24 hours, the slurries were filtered and the solution analysed by HPLC in order to determine the solubility at the various pH levels. For the buffer solutions, KCl/HCl was used for pH 1 and citrate/phosphate combinations for pH 3, 4.5 and 6.6 (10 mM). The pH of the solutions was also measured prior to HPLC analysis. XRPD analysis was carried out on the remaining solids after 24 hours of shaking.

Thermodynamic solubility experiments carried out in buffers pH 1, 3.0, 4.5 and 6.6 indicated the following result:

TABLE 16
Thermodynamic Solubility Studies

| Buffer pH | pH prior to analysis | Solubility (mg/mL) |
|---|---|---|
| 1 | 0.95 | 13.88 |
| 3 | 1.53 | 0.84 |
| 4.5 | 1.79 | 0.28 |
| 6.6 | 1.79 | 0.42 |

Competitive Slurry Experiments.

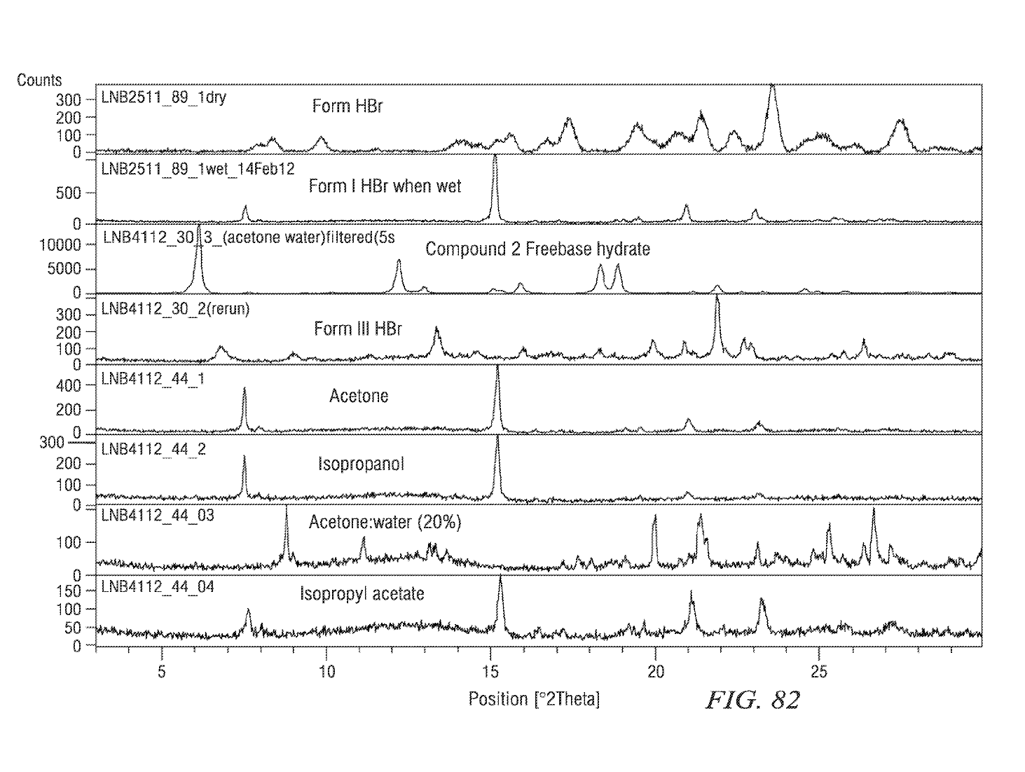

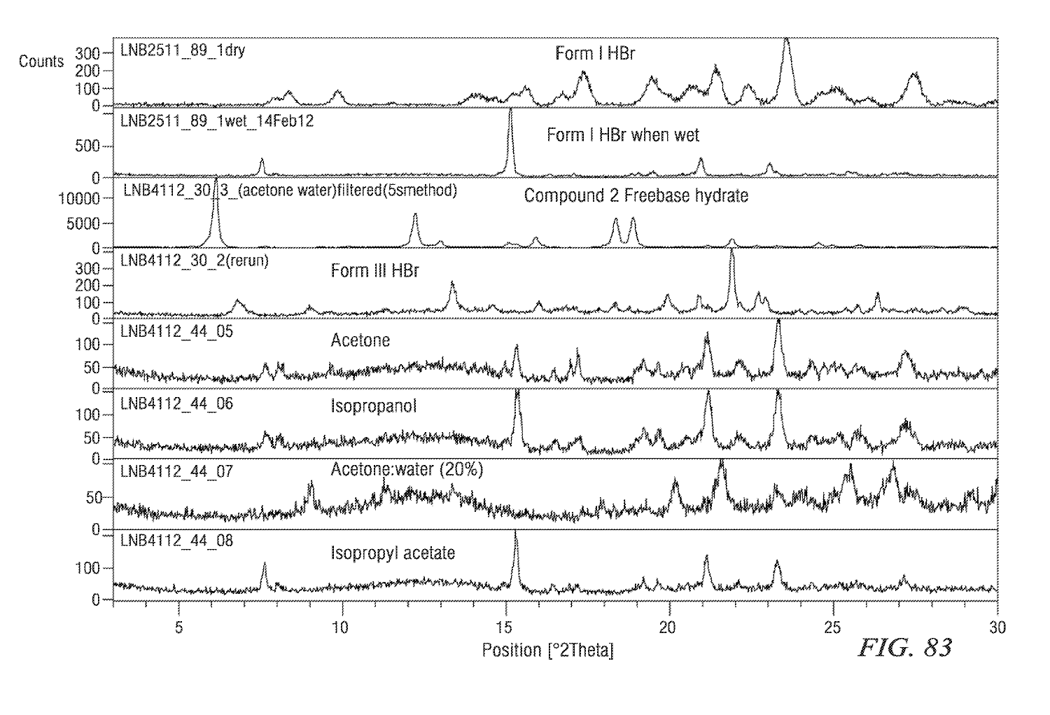

Competitive slurry experiments were set up in acetone, isopropanol, acetone:water (80:20) and isopropyl acetate at both room temperature (ca. 22° C.) and 60° C. Approximately 200 mg of each of Forms I and III material was placed into a vial and 4 mL of the appropriate solvent system was added to produce a slurry. For each experiment, the slurries were allowed to stir for ca. 3 days. Analysis by XRPD was then conducted to determine the form of the resultant solid. Competitive slurry experiments of Form I vs. Form III were carried out in 4 solvent systems and resulting solids were analysed by XRPD analysis (FIG. 82 and FIG. 83). Results are summarised in Table 17.

TABLE 17
Summary of results from competitive slurry experiments

| Ingoing Forms | Solvent | Temperature | Result |
|---|---|---|---|
| I and III | Acetone | RT (ca. 22° C.) | Form I |
| I and III | Acetone | 60° C. | Form I |
| I and III | Isopropanol | RT (ca. 22° C.) | Form I |
| I and III | Isopropanol | 60° C. | Form I |
| I and III | Acetone:water (80:20) | RT (ca. 22° C.) | Form VIII |
| I and III | Acetone:water (80:20) | 60° C. | Form VIII |
| I and III | Isopropyl acetate | RT (ca. 22° C.) | Form I |
| I and III | Isopropyl acetate | 60° C. | Form I |

From the competitive slurry experiments, Form I was found to be the thermodynamically most stable form in acetone, isopropanol and isopropyl acetate at both ambient and 60° C. In acetone: water (80:20), conversion to an unidentified form resulted (labelled as Form VIII).

Characterization of Form VIII.

An initial assessment of Form VIII, obtained from competitive slurry experiments of Forms I and III in acetone:water (80:20), was made in order to determine the nature of the form and evaluate whether it is consistent with freebase material or the HBr salt. The material resulting from the competitive slurry experiments appeared light yellow in colour. PLM analysis indicated birefringent material with no clearly defined morphology. After drying under vacuum for ca. 24 hours, the TGA/DTA indicated a weight loss of 5.2% from the outset followed by a second weight loss of 1.2%, with endotherms in the DTA trace at ca. 40° C. and ca. 96° C. A final endotherm was observed in the DTA trace at onset ca. 184° C. (peak ca. 194° C.). Very little change was observed by hotstage microscopy prior to the melt at ca. 197° C. DSC analysis indicated a broad endotherm starting from the outset (peak ca. 93° C.). followed by a second endotherm at peak ca. 140° C. and a third endotherm at onset ca. 178° C. (peak ca. 193° C.). Ion chromatography indicated a bromide content of 12.8% (approximately 1 equivalent).

In order to examine the effect of desolvating/dehydrating Form VIII, the material was heated to 150° C. in a TGA pan and XRPD analysis was then carried out. The polymorphic form appeared to remain the same. After heating to 150° C. and carrying out XRPD analysis, TGA analysis was again carried out on the same material and showed a weight loss of 6.0% from the outset followed by a second weight loss of 0.9%, with endotherms in the DTA trace at ca. 42° C. and 96° C. A final endotherm was observed in the DTA trace at onset ca. 186° C. (peak ca. 194° C.). The sample appeared to re-hydrate when exposed to atmospheric conditions. This would likely explain the consistency between the XRPD diffractograms before and after desolvation/dehydration.

Hydration Studies at 55° C.

Slurries were created using ca. 200 mg of Form I salt material in 2 mL of the appropriate solvent system. These were stirred at ca. 55° C. for 6 hours. The solvent systems used are listed in Table 18.

TABLE 18
Solvent Systems for Hydration Studies at 55° C.
Solvent System

Ethanol:Water (1%)
Ethanol:Water (2%)
Ethanol:Water (5%)
Ethanol:Water (10%)
IPA/Acetone (9:1):Water (1%)
IPA/Acetone (9:1):Water (2%)
IPA/Acetone (9:1):Water (5%)
IPA/Acetone (9:1):Water (10%)

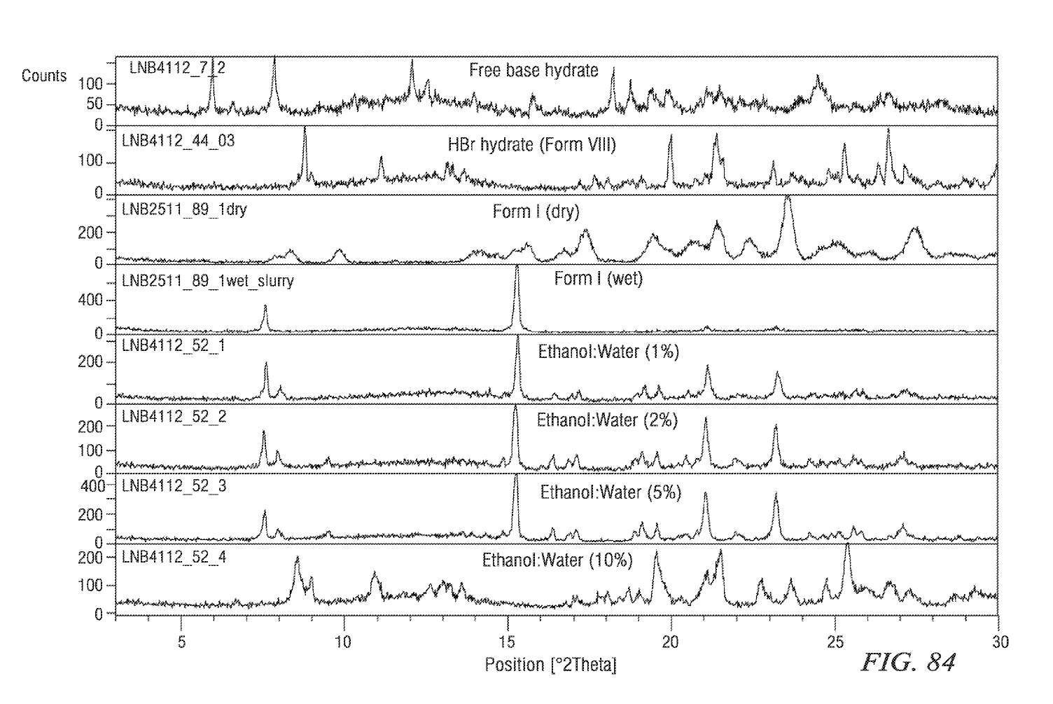

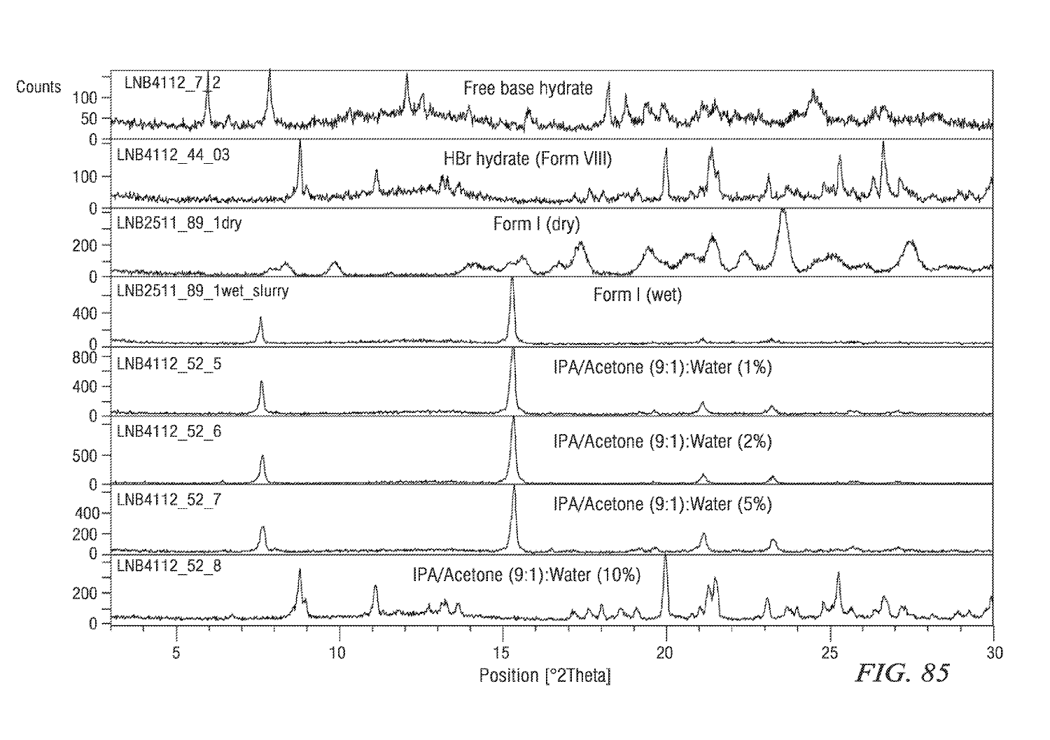

Following the hydration studies, material was analysed by XRPD to determine whether hydration or disproportionation had occurred at the various water activity levels. XRPD analysis of EtOH:Water samples revealed that, at 1, 2, and 5% water, the resultant diffractograms corresponded with the Form I input HBr salt material. At 10% water, the HBr hydrate was formed. The same pattern emerged for samples slurried in IPA/Acetone(9:1):Water mixtures, where at 1, 2, and 5% water the resultant diffractograms corresponded with the input Form I HBr salt, however, at 10% water, the HBr hydrate was obtained. The diffractograms can be seen in FIG. 84 and FIG. 85.

Hydration Studies at 15° C. and 35° C.

Slurries were created using ca. 200 mg of Form I salt material in 2 mL of the appropriate solvent system. These were stirred at ca. 15° C. and ca. 35° C. for 24 hours. The solvent systems used are listed in Table 19.

TABLE 19
Solvent Systems for Hydration Studies at 15° C. and 35° C.

| Solvent System | Temperature |
|---|---|
| Ethanol:Water (2%) | 35° C. |
| Ethanol:Water (2%) | 15° C. |
| IPA/Acetone (9:1):Water (2%) | 35° C. |
| IPA/Acetone (9:1):Water (2%) | 15° C. |

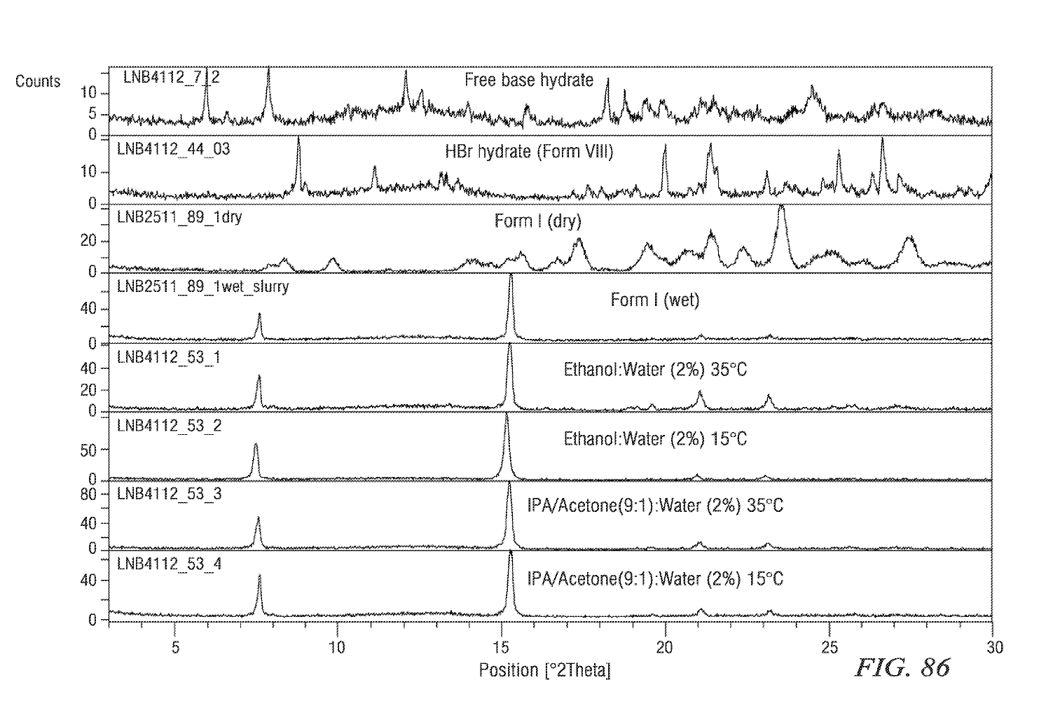

Following the hydration studies, XRPD analysis of samples revealed that the resultant diffractograms corresponded with the Form I HBr salt and hydration did not occur at the 2% water level. The diffractograms can be seen in FIG. 86.

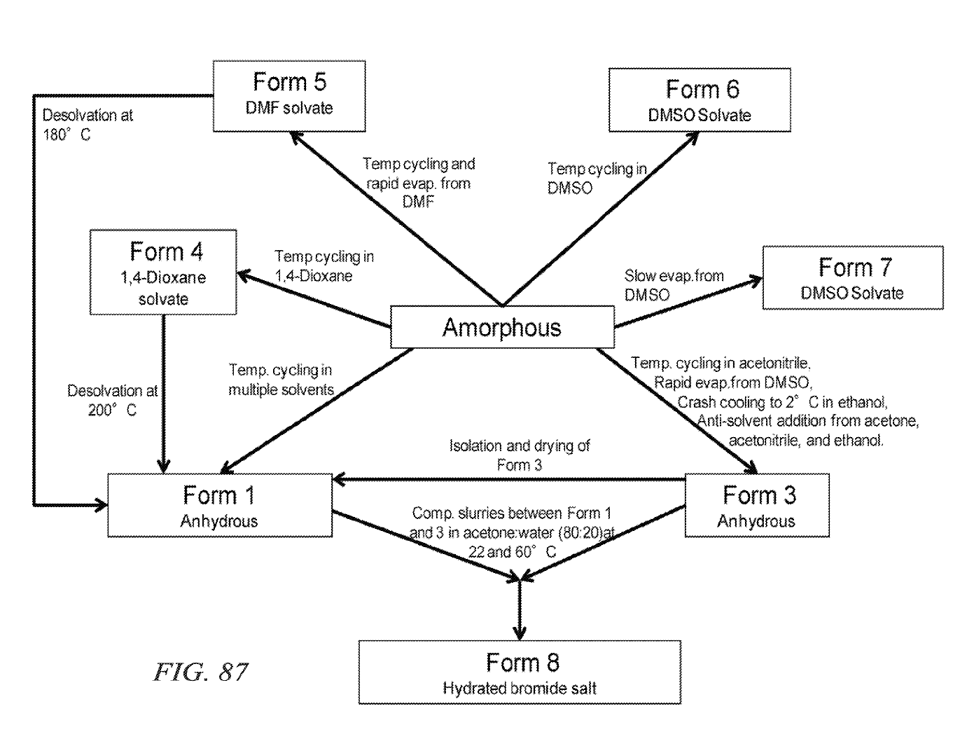

The results of the polymorph screen for the hydrobromide salt of compound 1 (compound 2 hydrobromide) is depicted in FIG. 87. Compound 2 hydrobromide exists in eight (8) different solid forms, including amorphous, anhydrous, solvated and hydrated forms. FIG. 87 illustrates the interconversion between several of the identified forms, with Form I exhibiting particular stability under a variety of conditions.

### Example 14

### Dog PK Study 1

Compound 1 free base and compound 2, as the Form I monohydrobromide (HBr) salt, were evaluated in a cross-over dog PK study. Compound 1 free base capsule consisted of compound 1 free base in Vitamin E TPGS and PEG 400 filled into a capsule. The Form I hydrobromide salt capsule consisted of Form I HBr alone filled into a capsule.

Compound 1 free base capsule and Form I HBr capsule were dosed orally at 28.5 and 24.5 mg/kg (as active) QD, respectively, to three fasted male non-naïve beagle dogs (body weight range: 10.1-10.8 kg) with a 5-day washout period. Approximately 5 mL of tap water was orally administered to encourage swallowing and ensure delivery of capsules into the stomach. Plasma samples were collected at pre-dose and 0.5, 1, 2, 4, 6, 8, 12 and 24 hours post dose. The plasma concentrations of compound 1 were determined by a liquid chromatography-tandem mass spectrometry (LC/MS/MS) method. The results are provided in Table 20.

When compound 1 is administered orally to fasted dogs at 24.5-28.5 mg/kg QD, compound 1 exposure (based on AUC and Cₘₐₓ) is significantly higher when drug is administered as the Form I HBr salt compared to the free base form.

TABLE 20
Mean pharmacokinetic parameters (% CV) of compound 1 in
fasted dogs (n = 3) receiving compound 1 (free base)
capsule and compound 2 (Form I hydrobromide) capsule orally

| | PK Values (% CV) | |
|---|---|---|
| | Compound 1 (Free | Compound 2 (Form I |
| Dog PK | Base) Capsule | Hydrobromide) Capsule |
| Parameters | 28.5 mg/kg | 24.5 mg/kg |
| Cₘₐₓ (ng/mL) | 120* | 1420 (47%) |
| Tₘₐₓ (h) | 1* (median) | 2 (median) |
| AUC₀₋₂₄ (ng · h/mL) | 278* | 5260 (74%) |
| T_{1/2} (h) | 2.1* | 2.4 |

*Dog #3 had emesis at 30 min post dose. At the 1 h time point, a partial capsule was found under its cage. Thus, data from dog #3 was not included in the calculation of PK parameters.

### Example 15

### Dog PK Study 2

Compound 1 free base and compound 2, as the Form I monohydrobromide (HBr) salt, were evaluated in a cross-over dog PK study in which male dogs were pre-treated with either pentagastrin (to decrease gastric pH) or famotidine (to increase gastric pH) prior to oral dosing to control gastric pH. In addition, the effect of food on the systemic exposure to compound 1 was also evaluated in dogs receiving Form I HBr with pentagastrin pre-treatment. Compound 1 free base capsule consisted of compound 1 free base in Vitamin E TPGS and PEG 400 filled into a capsule. The Form I hydrobromide salt capsule consisted of Form I HBr alone filled into a capsule.

Compound 1 free base and Form I HBr capsules were dosed orally at 30 mg/kg (as active) QD to three non-naïve male beagle dogs (body weight range: 9.6-10.5 kg) that were treated prior to dosing with 1) pentagastrin and fasted, 2) famotidine and fasted, or 3) pentagastrin and fed. There was a minimum 6-day washout between dosing. On the day of dosing under fed condition, dogs were given 60 gram of a high fat diet (Harlan Teklad 2027C) and allowed to consume all of the food within 15-20 minutes. The animals were given a 10-minute rest period and then the capsule doses were administered. Plasma samples were collected at pre-dose and 0.5, 1, 2, 4, 6, 8, 12 and 24 hours post dose. The results are provided in Table 21.

When compound 1 was administered orally to dogs at 30 mg/kg QD, compound 1 exposure was significantly higher when drug is administered as the HBr salt compared to the free base form under both low and high gastric pH conditions. In dogs receiving the free base capsules under high gastric pH condition, a 32- to 48-fold reduction in compound 1 exposure was observed compared to that under low pH condition. The effect of varying gastric pH on the systemic exposure to compound 1, measured as Cₘₐₓ and AUC, was greatly minimized when Form I HBr was administered. Administering Form I HBr with food resulted in an increase in Cₘₐₓ and AUC of compound 1 in dogs.

TABLE 21
Mean pharmacokinetic parameters (% CV) of compound
1 in dogs (n = 3) receiving compound 1 (free
base) capsule and compound 2 (Form I hydrobromide)
capsule orally following gastric pH adjustment treatment

| | PK Values (% CV) | | | |
|---|---|---|---|---|
| Treatment | Compound 1 (Free | | Compound 2 (Form I | |
| of Dogs | Base) Capsule | | Hydrobromide) Capsule | |
| Prior to | Cₘₐₓ | AUC₀₋₂₄ | Cₘₐₓ | AUC₀₋₂₄ |
| Dosing | (ng/mL) | (ng · h/mL) | (ng/mL) | (ng · h/mL) |
| Pentagastrin, | 1820 (20%) | 7810 (73%) | 2860 (39%) | 9450 (31%) |
| fasted | | | | |
| Famotidine, | 57 (24%) | 163 (3%) | 1180 (20%) | 4010 (54%) |
| fasted | | | | |
| Pentagastrin, | Not Dosed | Not Dosed | 3970 (28%) | 13900 (26%) |
| fed | | | | |

### Example 16

### Administration to Healthy Volunteers

The single dose pharmacokinetic (PK) profiles of Form I monohydrobromide (HBr) and Compound 1 (free base) were compared in a single centre non-randomised, open-label, single dose study in healthy male subjects. Subjects were screened for eligibility to participate in the study up to 28 days before dosing. The subjects were admitted to the clinical unit at approximately 09:00 in the morning on the day prior to dosing (Day-1) and remained on site until 24 h after each dose. Each subject attended a follow-up visit 4 to 6 days after the final dose.

One group of 12 subjects was dosed in an effort to obtain the data described above. Each subject received the following formulations in a crossover investigation. Dosing was separated by at least 7 days.
Regimen A: 150 mg Compound 1 (free base) capsule
Regimen B: 50 mg Compound 2 (Form I HBr) tablet formulation
Regimen C: ≦150 mg Compound 2 (Form I HBr) tablet formulation

All formulations were dosed in the morning, following an overnight fast. Subjects were allowed water up to 2 h before the scheduled dosing time and were provided with 240 mL of water at 2 h post-dose. Decaffeinated fluids were allowed ad libitum from lunch time on the day of dosing.

Subjects were provided with a light snack and then fasted from all food and drink (except water) for a minimum of 8 h on the day prior to dosing until approximately 4 h post-dose at which time lunch was provided. An evening meal was provided at approximately 9 h post-dose and an evening snack at approximately 14 h post-dose. On subsequent days, meals were provided at appropriate times.

Venous blood samples were withdrawn via an indwelling cannula or by venepuncture at the following times after dosing (hours): 0.5, 1, 1.5, 2, 4, 8, and 12.

The primary endpoint of the study was to compare the PK profiles of a formulation of Form I HBr with that of Compound 1 as a free base by measuring the following parameters: T_{lag}, Cₘₐₓ, Tₘₐₓ, AUC₍₀₋ₗₐₛₜ₎, AUC_{(0-inf)}, AUC_{% extrap}, Fᵣₑₗ, lambda-z, T_{1/2}el. The secondary endpoint of the study was to collect information about the safety and tolerability of Compound 1 (free base) and Compound 2 (Form I HBr salt) by assessing: physical examinations, safety laboratory tests, vital signs, electrocardiograms (ECGs), body temperature and AEs.

Plasma concentration data was tabulated and plotted for each subject for whom concentrations are quantifiable. PK analysis of the concentration time data obtained was performed using appropriate non-compartmental techniques to obtain estimates of the following PK parameters (where relevant).
T_{lag} the sampling time before the first quantifiable concentration of compound 1 in a concentration vs time profile
Cₘₐₓ the maximum observed plasma concentration
Tₘₐₓ the time from dosing at which Cₘₐₓ occurs
AUC₍₀₋ₗₐₛₜ₎ the area under the concentration vs time curve from time zero to the last measured time point
AUC_{(0-inf)} the area under the concentration vs time curve from time zero extrapolated to infinity
AUC_{% extrap} the percentage of AUC_{(0-inf)} accounted for by extrapolation
AUC₍₀₋ₜₐᵤ₎ the area under the concentration vs time curve within the dosing interval, estimated using the [linear or linear/log down] trapezoidal rule
AUC₍₀₋₂₄₎ the area under the concentration vs time curve from time zero to 24 hour post morning dose
RA relative accumulation
Fᵣₑₗ relative bioavailability of the test formulations compared with the reference formulation eg Regimens B, or C (test) compared with Regimen A (reference)
lambda-z slope of the regression line passing through the apparent elimination phase in a concentration vs time plot
T_{1/2}e1 the apparent elimination half-life

Assessment of dose proportionality, as appropriate eg Cₘₐₓ/D; AUC/D

Results.

The 150 mg Compound 2 HBr tablet formulation has demonstrated plasma exposures approximately three-fold greater than the Compound 1 (free base) capsule formulation, dose-for-dose, as well as greatly reduced PK variability. These data suggest that the tablet formulation can be administered at significantly lower oral doses to achieve higher and more predictable exposures in patients, compared with the current capsule formulation. Based on the unexpectedly improved PK properties of the tablet formulation, all subsequent clinical studies will be conducted with tablet formulations.

The Phase I dose escalation study will continue to enroll new patients with the free base capsule formulation at the current dose of 900 mg BID until the appropriate tablet formulations are available, at which time the dose escalation study will continue with the tablet formulation until maximum tolerated dose (MTD) is reached. Dose escalation with the tablet will begin at a dose of approximately 300 mg BID, which relates to the exposures seen at 900 mg BID with the free base capsule.

### Example 17

### Administration to Patients with Advanced EGFR Mutated NSCLC

In a phase I clinical study, escalating dose cohorts of patients with advanced mutant EGFR NSCLC were administered CO-1686 HBr (Compound 2) twice daily in continuous 21-day cycles. 4 dose levels were studied:
Dose level 1: 500 mg BID
Dose level 2: 750 mg BID
Dose level 3: 1000 mg BID
Dose level 4: 625 mg BID

In addition, patients who had previously been enrolled into the dosing cohorts of CO-1686 free base (Compound 1), and who were still receiving therapy, were switched from CO-1686 free base to CO-1686 HBr. CO-1686 was dosed in the fed state.

Interim Results.

Safety

Hyperglycemia emerged as the predominant dose limiting toxicity. To date, the frequency of hyperglycemia requiring intervention or dose reduction is as follows:

500 mg BID: 1 patient out of 6
625 mg BID: 1 patient out of 7
750 mg BID: 3 patients out of 9. In addition, one patient with pre-existing diabetes developed higher than normal plasma glucose levels in conjunction with CO-1686 treatment
1000 mg BID: 3 patients out of 6

No patients have permanently discontinued CO-1686 therapy because of uncontrolled plasma glucose. Plasma glucose levels normalize rapidly if CO-1686 is temporarily interrupted, or if hypoglycemic medication is co-administered (agents used successfully have included insulin, metformin, glipizide). Alternatively, plasma glucose levels may be controlled by reducing the dose of CO-1686.

There have been no signs of wild type EGFR inhibition such as skin rash and/or persistent diarrhea, including in patients with hyperglycemia, as well as in the overall population.

Pharmacokinetics

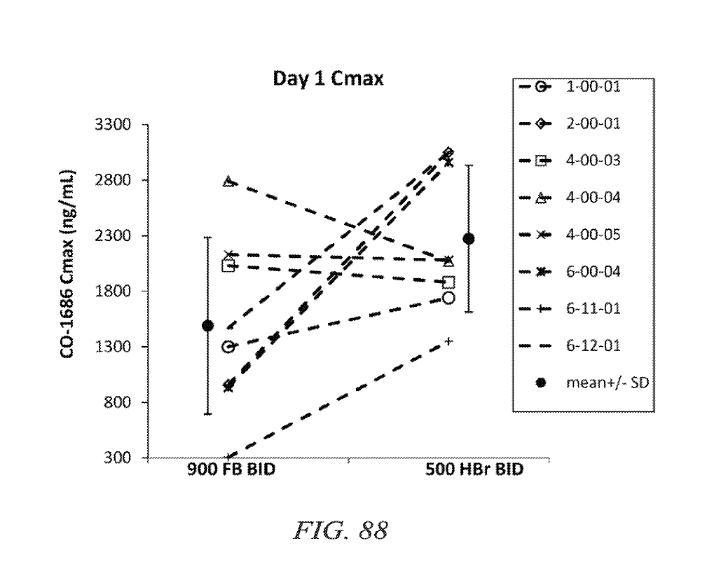

CO-1686 HBr showed increased absorption and thus, higher exposure than free base. The mean Cₘₐₓ and AUC₀₋₂₄ at 1000 mg CO-1686 HBr BID was approximately 3-fold of that at 900 mg free base BID. Comparative PK were available in 8 patients originally started CO-1686 treatment at 900 mg CO-1686 free base BID and then switched to 500 mg CO-1686 HBr BID (FIG. 88). Results suggest an enhanced absorption in low absorbers resulting in a higher mean value and lower variability.

Efficacy

Efficacy data are immature. Tumor shrinkage at cycle 2 has been observed in the majority of patients, including patients with T790M positive and T790M negative NSCLC. The majority of patients are still on treatment and have not achieved their best response. At the 1000 mg BID level, 2 patients achieved RECIST PR at cycle 2. Both patients with RECIST PRs also developed hyperglycemia requiring concomitant administration of metformin. Several patients with significant (>20%) tumor tumor target lesion shrinkage at cycle 2 have not developed abnormal plasma glucose levels.

One patient who had achieved stable disease on CO-1686 free base 900 mg bid experienced further tumor shrinkage after switching to CO-1686 HBr 500 mg BID resulting in a RECIST PR. In this patient, improved plasma exposure was observed with CO-1686 HBr compared with CO-1686 free base.

Recommended Phase 2 Dose

Based on available data, the dosing range will be 500 mg-2000 mg per day. Dosing is continuous, although dose reductions and delays are possible to manage adverse effects. The dosing regimen will likely be provided in doses of 250 mg BID-1000 mg BID, but once or three times daily regimens may be selected. Preferred doses will be 500 mg BID, 625 mg BID and 750 mg BID, as current data suggests they are associated with adequate plasma exposure, tumor shrinkage and an acceptable incidence of hyperglycemia.

1. A pharmaceutical dosage form comprising Compound 2: 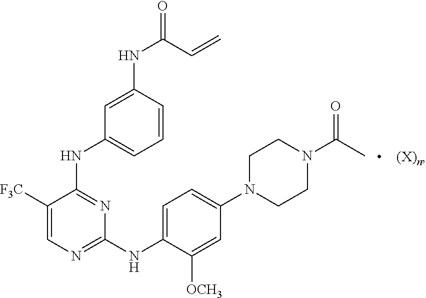
wherein:
n is 1 or 2; and
X is hydrobromic acid, benzenesulfonic acid, camphor sulfonic acid, 1,2-ethane disulfonic acid, hydrochloric acid, maleic acid, methanesulfonic acid, naphthalene-2-sulfonic acid, 1,5-naphthalene disulfonic acid, oxalic acid, 4-toluenesulfonic acid or 2,4,6-trihydroxybenzoic acid,
wherein the dosage form comprises Compound 2 in an amount of about 50 mg to about 1000 mg.

2. The pharmaceutical dosage form of claim 1, wherein X is hydrobromic acid.

3. The pharmaceutical dosage form of claim 2, wherein Compound 2 is a Form I hydrobromic acid salt characterized by one or more peaks in a powder X-ray diffraction pattern selected from those at about 17.39, about 19.45, about 21.41, about 23.56 and about 27.45 degrees 2-theta.

4. The pharmaceutical dosage form of claim 1, wherein the total daily dose of Compound 2 is about 500 mg to about 2000 mg.

5. The pharmaceutical dosage form of claim 4, wherein the dose of Compound 2 is 250 mg BID to 1000 mg BID.

6. The pharmaceutical dosage form of claim 5, wherein the dose of Compound 2 is 500 mg BID to 750 mg BID.

7. The pharmaceutical dosage form of claim 6, wherein the dose of Compound 2 is 500 mg BID.

8. The pharmaceutical dosage form of claim 6, wherein the dose of Compound 2 is 625 mg BID.

9. The pharmaceutical dosage form of claim 6, wherein the dose of Compound 2 is 750 mg BID.

10. The pharmaceutical dosage form of claim 5, wherein the dose of Compound 2 is 1000 mg BID.

11. The pharmaceutical dosage form of claim 5, wherein the dose of Compound 2 is 375 mg BID.

12. The pharmaceutical dosage form of claim 4, wherein the dose of Compound 2 is 375 mg TID.

13. The pharmaceutical dosage form of claim 1, wherein the dosage form comprises Compound 2 in an amount of about 50 mg to about 500 mg.

14. The pharmaceutical dosage form of claim 1, wherein the dosage form comprises Compound 2 in an amount of about 125 mg to about 250 mg.
